(19) United States
(12) Patent Application Publication
(10) Pub. No.: US 2024/0309072 A1
(43) Pub. Date: Sep. 19, 2024

(54) MULTIVALENT ANTI-SPIKE PROTEIN BINDING MOLECULES AND USES THEREOF

(71) Applicant: Regeneron Pharmaceuticals, Inc., Tarrytown, NY (US)

(72) Inventors: Nianshuang WANG, Cos Cob, CT (US); Yang SHEN, Scarsdale, NY (US); Alina BAUM, Pleasantville, NY (US); Christos KYRATSOUS, Irvington, NY (US); Chia-Yang LIN, Scarsdale, NY (US)

(73) Assignee: Regeneron Pharmaceuticals, Inc., Tarrytown, NY (US)

(21) Appl. No.: 18/589,384

(22) Filed: Feb. 27, 2024

Related U.S. Application Data
(60) Provisional application No. 63/487,408, filed on Feb. 28, 2023.

(51) Int. Cl. C07K 16/10; A61P 31/14

(52) U.S. Cl. CPC C07K 16/1003; A61P 31/14; C07K 2317/21; C07K 2317/24; C07K 2317/31; C07K 2317/35; C07K 2317/622

(57) ABSTRACT

The present disclosure provides multivalent anti-spike protein binding molecules, comprising multimerization moieties linked to anti-spike protein antigen-binding domains, that specifically bind to RBD regions of SARS-COV and/or SARS-COV-2. The present disclosure further relates to the methods of producing the multivalent anti-spike protein binding molecules, pharmaceutical compositions comprising of the multivalent anti-spike protein binding molecules, and methods of use of the multivalent anti-spike protein binding molecules to treat conditions associated with SARS-COV and SARS-COV-2 infections, such as COVID-19.

### 1. CROSS-REFERENCE TO RELATED APPLICATIONS

This application claims the priority benefit of U.S. provisional application No. 63/487,408, filed Feb. 28, 2023, the contents of which are incorporated herein in their entirety by reference thereto.

### 2. SEQUENCE LISTING

The instant application contains a Sequence Listing which has been submitted electronically in XML format and is hereby incorporated by reference in its entirety. Said XML Sequence Listing, created on Feb. 27, 2024 is named RGN-028US_SL.xml and is 643,930 bytes in size.

### 3. BACKGROUND

Severe acute respiratory syndrome coronavirus 2 (SARS-COV-2) is an enveloped, positive-sense, single-stranded RNA virus of the genus Betacoronavirus, which also includes SARS-COV, Middle East respiratory syndrome coronavirus (MERS-COV), human coronavirus (HCoV)-OC43, and HCoV-HKU1 (Jackson et al., 2022, Nat Rev Mol Cell Biol. 23(1): 3-20). SARS-COV-2 causes COVID-19, a potentially life-threatening disease which was first characterized in late 2019 and escalated into a global pandemic in early 2020.

SARS-COV-2 shares ˜80% identity with SARS-COV and both viruses rely on their interaction with the angiotensin-converting enzyme 2 (ACE2) for cellular entry, an enzyme expressed on the extracellular surface of many types of cells.

Currently, several vaccines against SARS-COV-2 are used to prevent manifestation of severe disease. However, vaccination rates vary among populations, and even in areas with high rates of vaccination, breakthrough infections leading to COVID-19 have been observed in individuals who have been immunized against SARS-COV-2. Previous SARS-COV-2 infections don't seem to provide a complete immunity against future infections either, as some individuals have been diagnosed with COVID-19 multiple times. Moreover, SARS-COV-2 infection in some individuals leads to a prolonged disease associated with persistence of one or more symptoms of COVID-19 for weeks to months after the clearance of infection. These observations underscore the serious population health threat posed by COVID-19 and the need to fight SARS-COV-2 infections with effective treatments.

Small molecule treatments such as Paxlovid—a combination of the oral antiviral drugs nirmatrelvir and ritonavir—can prevent hospitalization, yet they are associated with a ‘Paxlovid rebound’ effect in which the virus reemerges (Callaway, Nature (News), 11 Aug. 2022). On the other hand, biologic treatments, such as monoclonal antibodies, can directly blunt viral propagation, leading to robust and long-lasting therapeutic effects. However, due to rapid emergence of new SARS-COV-2 variants, antibodies isolated from patients are typically strain-specific, rendering them ineffective against certain SARS-COV-2 variants. Hence, there remains a need to develop neutralizing treatments that will be effective against SARS-COV-2.

### 4. SUMMARY

The present disclosure relates to multivalent antigen-binding molecules that bind to a coronavirus spike protein, generally referred to herein as “multivalent anti-spike protein binding molecules”, for inhibiting the interaction between coronaviruses and host cells. Multivalent anti-spike protein binding molecules of the disclosure typically comprise a plurality of anti-spike protein antigen-binding domains (ABDs) operably linked by one or more multimerization moieties. Multivalent anti-spike protein binding molecules of the disclosure are described in Section 6.2 and numbered embodiments 1 to 96.

The multivalent anti-spike protein binding molecules of the disclosure are typically tetravalent and comprise four spike protein ABDs, e.g., in the form of a Fab or an scFv. Spike protein ABDs of a multivalent anti-spike protein binding molecule of the disclosure can be monospecific (e.g., all ABDs bind to the same region of spike protein and optionally all have the same sequence) or multispecific (e.g., at least two of the ABDs bind to different regions or variants of spike protein and differ in sequence). Spike protein ABDs and spike protein ABD formats that are suitable for incorporation into multivalent anti-spike protein binding molecules of the disclosure are described in Sections 6.3 and 6.4, and numbered embodiments 3 to 15 and 48 to 61.

The multivalent anti-spike protein binding molecules of the disclosure include one or more multimerization moieties, for example one or more multimerization moieties that comprise or consist of an Fc domain. Multimerization moieties suitable for incorporation into the multivalent anti-spike protein binding molecules of the disclosure are described in Section 6.5 and numbered embodiments 28 to 32 and 36 to 39.

Two or more components of the multivalent anti-spike binding protein binding molecules of the disclosure can be connected to one another by a linker, e.g., a peptide linker. By way of example and without limitation, linkers can be used to connect a spike protein ABD to a multimerization moiety. Linkers suitable for incorporation into the multivalent anti-spike binding protein binding molecules of the disclosure are described in Section 6.6 and numbered embodiments 44 to 47.

The multivalent anti-spike protein binding molecules of the disclosure can comprise a linker that is a hinge region. Suitable hinge sequences for incorporation into multivalent anti-spike protein binding molecules of the disclosure are described in Section 6.6.1 and numbered embodiments 40 to 43.

The present disclosure further provides nucleic acids encoding the multivalent anti-spike protein binding molecules of the disclosure, host cells engineered to express the multivalent anti-spike protein binding molecules of the disclosure, and recombinant methods for the production of the multivalent anti-spike protein binding molecules of the disclosure. Such nucleic acids, host cells and production methods are described in Section 6.7 and numbered embodiments 97 to 99.

The present disclosure further provides pharmaceutical compositions comprising the multivalent anti-spike protein binding molecules of the disclosure as well as therapeutic indications and methods of use. Pharmaceutical compositions are described in Section 6.8 and numbered embodiment 100. Methods of use of the multivalent anti-spike protein binding molecules are described in Section 6.9 and numbered embodiments 101 to 113.

Other features and advantages of aspects of the multivalent anti-spike protein binding molecules of the present disclosure will become apparent from the following more detailed description, taken in conjunction with the accompanying drawings.

### 5. BRIEF DESCRIPTION OF THE FIGURES

FIGS. 1A-1D show exemplary multivalent anti-spike protein binding molecules of the disclosure. FIG. 1A represents a monospecific tetravalent 2×2 N-Fab alternative format (“AF”) antibody construct with identical Fab arms, each comprising two serially linked Fab moieties, connected to the N-terminal end of an Fc moiety. FIG. 1B represents a bispecific tetravalent 2×2 N-Fab AF construct with different Fab arms, each comprising two serially linked Fab moieties, connected to the N-terminal end of an Fc moiety. FIG. 1C represents a monospecific tetravalent 2×2 C-Fab AF constructs with identical Fab arms and comprising Fab moieties connected to the N-terminal ends of Fc moieties, which are also linked to Fab moieties on their C-terminal ends. FIG. 1D represents a bispecific tetravalent 2×2 C-Fab AF constructs with different Fab arms and comprising Fab moieties connected to the N-terminal ends of Fc moieties, which are also linked to Fab moieties on their C-terminal ends.

FIGS. 2A-2B show neutralization activities of different 2×2 N-Fab AF constructs against SARS-COV-2 pseudovirus variants. An individual Fab arm from REGN10987, 14315, 10933, 14256 and 10985 has been used to pair with itself or others. FIG. 2A displays the pseudovirus neutralization IC50 values of constructs grouped by Fab arms against the D614G variant, whereas FIG. 2B displays the neutralization IC50 values against the BA.2 variant. Closed circles represent the 2×2 N-Fab AF constructs that have identical Fab arms, hence are monospecific. Open circles represent the 2×2 N-Fab AF constructs that have different Fab arms, thus are bispecific. The closed triangle in each Fab group represents the parental IgG control with that Fab.

FIGS. 3A-3B show neutralization activities of different 2×2 C-Fab AF constructs against SARS-COV-2 pseudovirus variants. FIG. 3A displays the pseudovirus neutralization IC50 values of constructs against the D614G variant, whereas FIG. 3B displays the neutralization IC50 values of the same constructs against the BA.2 variant, grouped by Fab arms. Closed circles represent the 2×2 C-Fab AF constructs that have identical Fab arms, hence are monospecific. Open circles represent the 2×2 C-Fab AF constructs that have different Fab arms, thus are bispecific. The closed triangle in each Fab group represents the parental IgG control with that Fab.

FIGS. 4A-4B show dose-dependent neutralization activities of REGN14287-based tetravalent AF molecules against SARS-COV-2 pseudovirus Omicron variant BQ.1. FIG. 4A shows the neutralization activity in percent neutralization against BQ.1 in dose titration of COVAF-46-51 in 2×2 N-Fab with the bivalent parental IgG REGN14287 control and the cocktail combination of REGN10933/REGN10987. FIG. 4B shows the neutralization activity in percent neutralization against BQ. 1 in dose titration of COVAF-52-57 in 2×2 C-Fab with the bivalent parental IgG REGN14287 control and the cocktail combination of REGN10933/REGN10987.

### 6. DETAILED DESCRIPTION

### 6.1. Definitions

As used herein, the following terms are intended to have the following meanings:

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain comprises a heavy chain variable region (abbreviated herein as VH) and a heavy chain constant region. The heavy chain constant region comprises three domains, CH1, CH2 and CH3. Each light chain comprises a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelized antibodies, chimeric antibodies, bispecific or multispecific antibodies and anti-idiotypic (anti-id) antibodies. The antibodies can be of any isotype/class (e.g., IgG, IgE, IgM, IgD, IgA and IgY) or subclass (e.g., IgG1, IgG2, IgG3, IgG4, IgA1 and IgA2). Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding domain or amino-terminus of the antibody. The N-terminus is a variable region and at the C-terminus is a constant region; the CH3 and CL domains represent the carboxy-terminus of the heavy and light chain, respectively, of natural antibodies. For convenience, and unless the context dictates otherwise, the reference to an antibody also refers to antibody fragments as well as engineered antibodies that include non-naturally occurring antigen-binding domains and/or antigen-binding domains having non-native configurations.

Antigen Binding Molecule or ABM: The term “antigen binding molecule” or “ABM” as used herein refers to a molecule (e.g., an assembly of multiple polypeptide chains) comprising two half antibodies. Typically, each half antibody comprises at least one antigen-binding domain. In some embodiments, the antigen is a coronavirus spike protein; hence, the ABMs of the disclosure are generally referred to as “anti-spike protein binding molecules.” The ABMs of the disclosure can be monospecific or multispecific (e.g., bispecific). The antigen binding domain in monospecific binding molecules all bind to the same epitope whereas multispecific binding molecules have at least two antigen-binding sites that bind to different epitopes, which can be on the same or different molecules (e.g., different spike protein variants).

Antigen-binding domain: The term “antigen-binding domain” or “ABD” as used herein refers to a portion of an antibody or antibody fragment that has the ability to bind to an antigen non-covalently, reversibly and specifically. Examples of an antibody fragment that can comprise an ABD include, but are not limited to, a single-chain Fv (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)₂ fragments) and engineered proteins comprising one or more portions of an antibody (e.g., an scFv). Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136).

Associated: The term “associated” in the context of a multivalent anti-spike protein binding molecule refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional multivalent anti-spike protein binding molecule. Examples of associations that might be present in a multivalent anti-spike protein binding molecule of the disclosure include (but are not limited to) associations between Fc domains to form an Fc region (e.g., as described in Section 6.5.1).

Bivalent: The term “bivalent” as used herein refers to a binding molecule comprising two antigen binding domains, whether in the same polypeptide chain or on different polypeptide chains.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Constant domain: The terms “constant domain” refers to a CH1, CH2, CH3 or CL domain of an immunoglobulin.

The term “CH1 domain” refers to the heavy chain constant region linking the variable domain to the hinge in a heavy chain constant domain. In some embodiments, the term “CH1 domain” refers to the region of an immunoglobulin molecule spanning amino acids 118 to 215 (EU numbering). The term “CH1 domain” encompasses wildtype CH1 domains as well as variants thereof (e.g., non-naturally-occurring CH1 domains or modified CH1 domains). For example, the term “CH1 domain” includes wildtype IgG1, IgG2, IgG3 and IgG4 CH1 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH1 domains include CH1 domains with mutations that modify a biological activity of an antibody, such as ADCC, CDC or half-life.

The term “CH2 domain” refers to the heavy chain constant region linking the hinge to the CH3 domain in a heavy chain constant domain. In some embodiments, the term “CH2 domain” refers to the region of an immunoglobulin molecule spanning amino acids 238 to 340 (EU numbering). The term “CH2 domain” encompasses wildtype CH2 domains as well as variants thereof (e.g., non-naturally-occurring CH2 domains or modified CH2 domains). For example, the term “CH2 domain” includes wildtype IgG1, IgG2, IgG3 and IgG4 CH2 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH2 domains include CH2 domains with mutations that modify a biological activity of an antibody, such as ADCC, CDC, purification, dimerization and half-life.

The term “CH3 domain” refers to the heavy chain constant region that is C-terminal to the CH2 domain in a heavy chain constant domain. In some embodiments, the term “CH3 domain” refers to the region of an immunoglobulin molecule spanning amino acids 341 to 447 (EU numbering). The term “CH3 domain” encompasses wildtype CH3 domains as well as variants thereof (e.g., non-naturally-occurring CH3 domains or modified CH3 domains). For example, the term “CH3 domain” includes wildtype IgG1, IgG2, IgG3 and IgG4 CH3 domains and variants thereof having 1, 2, 3, 4, 5, 1-3, 1-5, 3-5 and/or at most 5, 4, 3, 2, or 1 mutations, e.g., substitutions, deletions and/or additions. Exemplary CH3 domains include CH3 domains with mutations that modify a biological activity of an antibody, such as ADCC, CDC, purification, dimerization and half-life.

The term “CL domain” refers to the constant region of an immunoglobulin light chain. The term “CL domain” encompasses wildtype CL domains (e.g., kappa or lambda light chain constant regions) as well as variants thereof (e.g., non-naturally-occurring CL domains or modified CL domains). For example, the term “CL domain” includes wildtype kappa and lambda constant domains and variants thereof having 1, 2, 3, 4, 5, 1-3, 1-5, 3-5 and/or at most 5, 4, 3, 2, or 1 mutations, e.g., substitutions, deletions and/or additions.

COVID-19: The term “COVID-19” is the abbreviation of “Coronavirus disease 2019” and refers to the infectious disease caused by SARS-COV-2 infection. Patients with COVID-19 may experience a wide range of symptoms ranging from mild to severe, which may include but are not limited to, fever, chills, cough, shortness of breath, difficulty breathing, fatigue, muscle aches, body aches, headache, loss of smell, loss of taste, sore throat, congestion, runny nose, nausea, and diarrhea.

EC50: The term “EC50” refers to the half maximal effective concentration of a molecule (such as a multivalent anti-spike protein binding molecule) which induces a response halfway between the baseline and maximum after a specified exposure time. The EC50 essentially represents the concentration of a multivalent anti-spike protein binding molecule where 50% of its maximal effect is observed. In certain embodiments, the EC50 value equals the concentration of a multivalent anti-spike protein binding molecule that gives half-maximal virus or pseudovirus neutralization in an assay as described in Section 8.1.2.

Epitope: An epitope, or antigenic determinant, is a portion of an antigen recognized by an antibody or a fragment thereof, e.g., an antigen-binding domain. An epitope can be linear or conformational.

Fab: The term “Fab” refers to a pair of polypeptide chains, the first comprising a variable heavy (VH) domain of an antibody operably linked (typically N-terminal to) to a first constant domain (referred to herein as C1), and the second comprising variable light (VL) domain of an antibody N-terminal operably linked (typically N-terminal) to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps that facilitate correct VH and VL pairings. For example, it is possible to replace the CH1 and CL domain pair in a Fab with a CH3-domain pair to facilitate correct modified Fab-chain pairing in heterodimeric molecules. It is also possible to reverse CH1 and CL, so that the CH1 is attached to VL and CL is attached to the VH, a configuration generally known as Crossmab. The term “Fab” encompasses single chain Fabs.

Fc Domain and Fc Region: The term “Fc domain” refers to a portion of the heavy chain that pairs with the corresponding portion of another heavy chain. In some embodiments an Fc domain comprises a CH2 domain followed by a CH3 domain, with or without a hinge region N-terminal to the CH2 domain. The term “Fc region” refers to the region formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. When present on a single polypeptide chain (e.g., a scFv), the VH and be N-terminal or C-terminal to the VL.

Half Antibody: The term “half antibody” refers to a molecule that comprises at least Fc domain and can associate with another molecule comprising an Fc domain through, e.g., a disulfide bridge or molecular interactions (e.g., knob-in-hole interactions between Fc heterodimers). A half antibody can be composed of one polypeptide chain or more than one polypeptide chains (e.g., a heavy chain and a light chain).

Hinge: The term “hinge”, as used herein, is intended to include the region of consecutive amino acid residues that connect the C-terminus of the CH1 to the N-terminus of the CH2 domain of an immunoglobulin. Several amino acids of the N-terminus of the CH2 domain, which are coded by the CH2 exon, are also considered part of the “lower hinge”. Without being bound by any one theory, amino acids of the hinge region of IgG1, IgG2 and IgG4 have been characterized as comprising 12-15 consecutive amino acids encoded by a distinct hinge exon, and several N-terminal amino acids of the CH2 domain (encoded by the CH2 exon) (Brekke et al., 1995, Immunology Today 16(2):85-90). On the other hand, IgG3 comprises a hinge region consisting of four segments: one upper segment resembling the hinge region of IgG1, and 3 segments that are identical amino acid repeats unique to IgG3.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey, or human cell line, for example HKB11 cells, PER.C6 cells, HEK cells or CHO cells.

Immunoglobulin: The term “immunoglobulin” (Ig) refers to a class of structurally related glycoproteins consisting of two pairs of polypeptide chains, one pair of light (L) chains and one pair of heavy (H) chains, which may all four be inter-connected by disulfide bonds. The structure of immunoglobulins has been well characterized. See for instance Fundamental Immunology Ch. 7 (Paul, W., ed., 2nd ed. Raven Press, N. Y. (1989)). Each heavy chain typically comprises a heavy chain variable region (abbreviated herein as VH or VH) and a heavy chain constant region (CH or CH). The heavy chain constant region typically comprises three domains, CH1, CH2, and CH3. The CH1 and CH2 domains are linked by a hinge. The Fc portion comprises at least the CH2 and CH3 domains.

Typically, the numbering of amino acid residues of immunoglobulins is according to IMGT, Sequences of Proteins of Immunological Interest, 5th Ed. Public Health Service, National Institutes of Health, Bethesda, MD. (1991), or by the EU numbering system of Kabat (also known as “EU numbering” or “EU index”), e.g., as in Kabat et al. Sequences of Proteins of Immunological interest. 5th ed. US Department of Health and Human Services, NIH publication No. 91-3242 (1991).

Linker: The term “linker” as used herein refers to a connecting peptide between two moieties. For example, a linker can connect a spike protein ABD to an Fc domain.

Multivalent: The term “multivalent” as used herein refers to an antigen-binding molecule comprising two or ABDs, on one, two or more polypeptide chains.

Neutralizing, Blocking: A “neutralizing” or “blocking” spike protein ABD refers to an ABD, whose binding to spike protein inhibits an activity of the spike protein to any detectable degree, e.g., inhibits the ability of spike protein to bind to a receptor such as ACE2, to be cleaved by a protease such as TMPRSS2, or to mediate viral entry into a host cell or viral reproduction in a host cell.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of a multivalent anti-spike protein binding molecule of the disclosure, separate components (e.g., a spike protein ABD and an Fc domain) can be operably linked directly or through peptide linker sequences. In the context of a nucleic acid encoding a fusion protein, such as a multivalent anti-spike protein binding molecule of the disclosure, “operably linked” means that the two nucleic acids are joined such that the amino acid sequences encoded by the two nucleic acids remain in-frame.

Polypeptide, Peptide and Protein: The terms “polypeptide”, “peptide” and “protein” are used interchangeably herein to refer to a polymer of amino acid residues.

Recognize: The term “recognize” as used herein refers to an antibody or antibody fragment (e.g., a spike protein ABD) that finds and interacts (e.g., binds) with its epitope.

Single Chain Fab or scFab: The term “single chain Fab” or “scFab” as used herein refers to a polypeptide chain comprising the VH, CH1, VL and CL domains of antibody, where these domains are present in a single polypeptide chain.

Single Chain Fv or scFv: The term “single-chain Fv” or “scFv” as used herein refers to ABDs comprising the VH and VL domains of an antibody, wherein these domains are present in a single polypeptide chain. Preferably, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds. (1994), Springer-Verlag, New York, pp. 269-315. The VH and VL can be arranged in the N-to C-terminal order VH-VL or VL-VH, typically separated by a linker.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

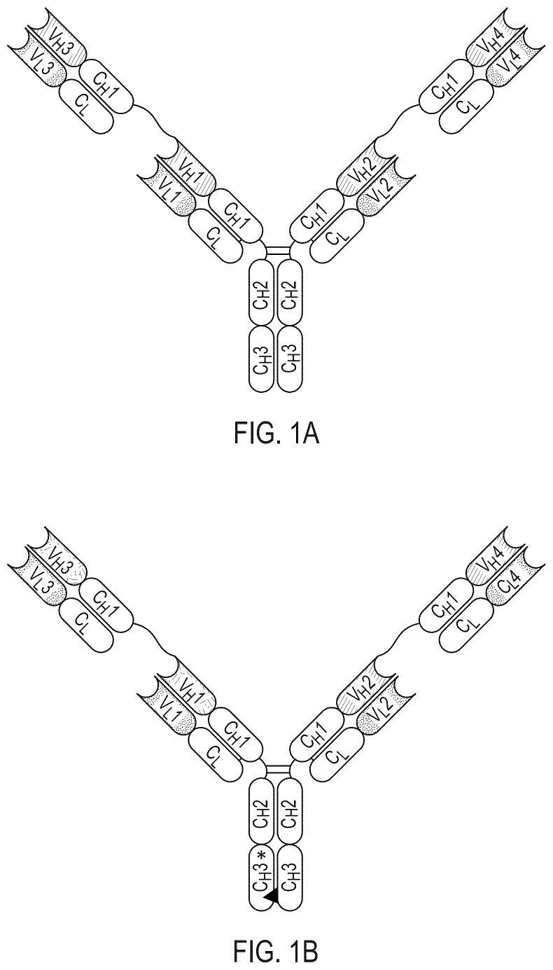

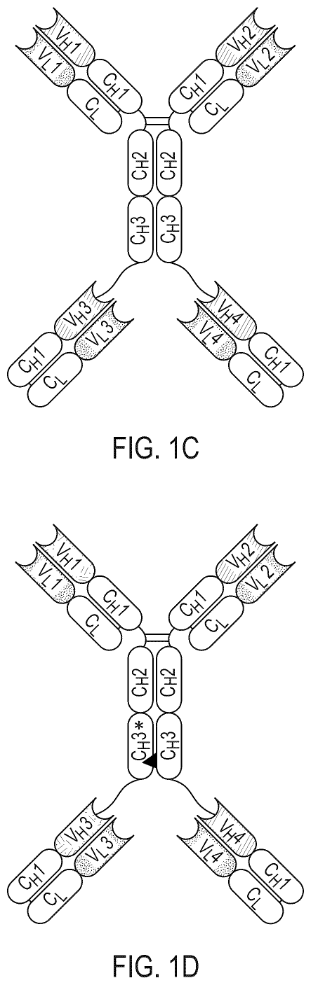

Tetravalent: The term “tetravalent” as used herein in relation to an antigen-binding molecule refers to an antigen-binding molecule comprising four ABDs. In some embodiments, a tetravalent anti-spike protein binding molecule refers to an anti-spike protein binding molecule comprising four spike protein ABDs. The four spike protein ABDs can be the same or different. In some embodiments, a tetravalent spike protein binding molecule has the configuration depicted in any one of FIGS. 1A-1D.

Treat, Treatment, Treating: As used herein, the terms “treat”, “treatment” and “treating” refer to the reduction or amelioration of the progression, severity and/or duration of a disease or condition and/or the amelioration of one or more symptoms (preferably, one or more discernible symptoms) of a disease or condition resulting from the administration of one or more multivalent anti-spike protein binding molecules of the disclosure.

In some embodiments, the disease or condition is caused by a coronavirus infection, for example SARS-COV or SARS-COV-2, for example COVID-19. In some embodiments, the disease or condition is any other ailment associated with SARS-COV or SARS-COV-2 infection, or similar infections. With reference to these diseases and conditions, the terms “treat”, “treatment” and “treating” refer to the reduction or amelioration of the progression, severity and/or duration of the disease or the amelioration of one or more symptoms (preferably, one or more discernible symptoms) of a disease resulting from the administration of one or more multivalent anti-spike protein binding molecules of the disclosure. In specific embodiments, the terms “treat”, “treatment” and “treating” refer to the amelioration of at least one measurable physical parameter of COVID-19, such as blood oxygen saturation levels, not necessarily discernible by the patient. In other embodiments the terms “treat”, “treatment” and “treating” refer to the inhibition of the progression of COVID-19, either physically by, e.g., stabilization of a discernible symptom, physiologically by, e.g., stabilization of a physical parameter, or both. In other embodiments the terms “treat”, “treatment” and “treating” refer to the reduction or elimination of infection.

### 6.2. Multivalent Anti-Spike Protein Binding Molecules

Disclosed herein are multivalent antigen-binding molecules that bind to a coronavirus spike protein, generally referred to herein as “multivalent anti-spike protein binding molecules.”

The anti-spike protein binding molecules of the disclosure typically have a valency of greater than two. In some embodiments, the anti-spike protein binding molecules include at least four antigen-binding domains (ABDs) that bind to spike protein. In some embodiments, the anti-spike protein binding molecules are tetravalent.

The anti-spike protein binding molecules can be monospecific or multispecific.

In some embodiments, the anti-spike protein binding molecules are monospecific, wherein all the ABDs bind to the same epitope, and optionally all have the same binding sequences.

In other embodiments, the anti-spike protein binding molecules are multispecific, wherein the ABDs bind to two or more different epitopes. In some embodiments, the anti-spike protein binding molecules are bispecific, with antigen-binding domains that bind to two different epitopes. In some embodiments, the two different epitopes are spike protein epitope, whether two different epitopes in the same spike protein, two different epitopes present in two spike protein variants, or a combination thereof.

In some aspects, a tetravalent anti-spike protein binding molecule comprises four spike protein ABDs, e.g., in the form of a Fab or an scFv as described in Section 6.4.1 or Section 6.4.2, respectively.

The ABDs of a multivalent anti-spike protein binding molecule of the disclosure are operably linked by one more multimerization moieties and may be present one or more polypeptide chains. Exemplary multimerization moieties are described in Section 6.5.

In some embodiments, the one or more multimerization moieties are a pair of Fc domains that associate to form an Fc dimer. The Fc dimer can be a homodimer, e.g., as depicted in FIG. 1A or FIG. 1C, or a heterodimer, e.g., as depicted in FIG. 1B or FIG. 1D. Each polypeptide chain comprising an Fc domain and associated polypeptide chains (e.g., light chain polypeptide chains) are referred to herein as “half antibodies”.

Thus, the present disclosure provides a multivalent spike protein binding molecule comprising two half antibodies. In some embodiments, each half antibody comprises at least two ABDs.

In certain aspects, the present disclosure provides a tetravalent multivalent spike protein binding molecule comprising two half antibodies. In some embodiments, each half antibody comprises at least two ABDs.

In some embodiments, the present disclosure provides a multivalent spike protein binding molecule, sometimes referred to herein as a Type 1 multivalent spike protein binding molecule or Type 1 ABM, comprising two half antibodies:
a) a first half antibody comprising:
   i) a first ABD, typically comprising a first VH and a first VL;
   ii) an optional linker;
   iii) a second ABD, typically comprising a second VH and a second VL;
   iv) an optional linker (e.g., a hinge domain); and
   v) a first Fc domain; and
b) a second half antibody comprising:
   i) a third ABD, typically comprising a third VH and a third VL;
   ii) an optional linker;
   iii) a fourth ABD, typically comprising a fourth VH and a fourth VL;
   iv) an optional linker (e.g., a hinge domain); and
   v) a second Fc domain.

In further embodiments, the present disclosure provides a multivalent spike protein binding molecule, sometimes referred to herein as a Type 2 multivalent spike protein binding molecule or a Type 2 ABM, comprising two half antibodies:
a) a first half antibody comprising:
   i) a first ABD, typically comprising a first VH and a first VL;
   ii) an optional linker (e.g., a hinge domain);
   iii) a first Fc domain;
   iv) an optional linker; and
   v) a second ABD, typically comprising a second VH and a second VL; and
b) a second half antibody comprising:
   i) a third ABD, typically comprising a third VH and a third VL;
   ii) an optional linker (e.g., a hinge domain);
   iii) a second Fc domain;
   iv) an optional linker; and
   v) a fourth ABD, typically comprising a fourth VH and a fourth VL.

In both the Type 1 and Type 2 ABMs, the first, second, third and fourth ABDs can be the same or different. In some embodiments, the first, second, third and fourth ABDs are the same. In other embodiments, the first and second ABDs are the same and the third and fourth ABDs are the same (but different from the first and second ABDs). In further embodiments, the first and third ABDs are the same and the second and fourth ABDs are the same (but different from the first and third ABDs).

In some embodiments, two of the ABDs or all four of the ABDs are scFvs, and thus the VH and the VLs of such ABDs are on the same polypeptide chain. Exemplary scFv structures are described in Section 6.4.2.

In some embodiments, two of the ABDs or all four of the ABDs are Fabs. Optionally, the Fabs are not single chain Fabs, and thus the VH and the VLs of such ABDs are on the separate polypeptide chains. In some embodiments, the VH of an ABD is on the same polypeptide chain as the Fc domain of the half antibody comprising the ABD, and the VL is on a separate polypeptide chain. In some embodiments, the VL of an ABD is on the same polypeptide chain as the Fc domain of the half antibody comprising the ABD, and the VH is on a separate polypeptide chain. The Fab can be in native immunoglobulin conformation and the polypeptide chain comprising the VH can further comprise the CH1 domain of the Fab, or can be domain exchanged and thus the polypeptide chain comprising the VH can further comprise the CL domain of the Fab. Exemplary Fab structures (including domain exchange structures) are described in Section 6.4.1.

Exemplary linkers for the multivalent spike protein binding molecules of the disclosure, including the linkers for the Type 1 and Type 2 multivalent spike protein binding molecules of the disclosure, are described in Section 6.6.

In some embodiments, the multivalent spike protein binding molecule is monospecific, wherein all ABDs bind to the same epitope. The ABDs in a monospecific multivalent spike protein binding molecule may all comprise the same CDR sequences or the same VH and VL sequences. In some embodiments, the ABDs of a monospecific multivalent spike protein binding molecule are configured as shown in FIG. 1A or FIG. 1C. FIG. 1A depicts a monospecific Type 1 ABM and FIG. 1C depicts a monospecific Type 2 ABM. In FIG. 1A, the Fab comprising VH3-VL3 represents the first ABD of a Type 1 ABM, the Fab comprising VH1-VL1 represents the second ABD of a Type 1 ABM, the Fab comprising VH4-VL4 represents the third ABD of a Type 1 ABM, and the Fab comprising VH2-VL2 represents the fourth ABDs of a Type 1 ABM. In FIG. 1B, the Fab comprising VH1-VL1 represents the first ABD of a Type 2 ABM, the Fab comprising VH3-VL3 represents the second ABD of a Type 2 ABM, the Fab comprising VH2-VL2 represents the third ABD of a Type 2 ABM, and the Fab comprising VH4-VL4 represents the fourth ABDs of a Type 2 ABM. Monospecific Type 1 and Type 2 ABMs may include two identical half antibodies, and thus the association of the two half antibodies forms an Fc homodimer. Suitable Fc domains are described in Section 6.5.1. The Fc domains can include mutations that reduce effector function as compared to wild type Fc domains, e.g., as described in Section 6.5.1.1.

In some embodiments, the multivalent spike protein binding molecule is multispecific, wherein at least two ABDs bind to different epitopes. At least two ABDs in a multispecific multivalent spike protein binding molecule comprise different CDR sequences. In some embodiments, the multispecific multivalent spike protein binding molecule is tetravalent, with a first pair of ABDs sharing the same CDR sequences or the same VH and VL sequences and a second pair of ABDs sharing the CDR sequences or the same VH and VL sequences (which differ from the CDR sequences of the first pair of ABDs). In some embodiments, the ABDs of a multispecific multivalent spike protein binding molecule are configured as shown in FIG. 1B or FIG. 1D. FIG. 1B depicts a multispecific Type 1 ABM and FIG. 1D depicts a multispecific Type 2 ABM. In FIG. 1B, the Fab comprising VH3-VL3 represents the first ABD of a Type 1 ABM, the Fab comprising VH1-VL1 represents the second ABD of a Type 1 ABM, the Fab comprising VH4-VL4 represents the third ABD of a Type 1 ABM, and the Fab comprising VH2-VL2 represents the fourth ABDs of a Type 1 ABM. In FIG. 1D, the Fab comprising VH1-VL1 represents the first ABD of a Type 2 ABM, the Fab comprising VH3-VL3 represents the second ABD of a Type 2 ABM, the Fab comprising VH2-VL2 represents the third ABD of a Type 2 ABM, and the Fab comprising VH4-VL4 represents the fourth ABDs of a Type 2 ABM. Multispecific Type 1 and Type 2 ABMs may include two identical half antibodies or two different antibodies, and thus the association of the two half antibodies may form an Fc homodimer or an Fc heterodimer. Suitable Fc domains are described in Section 6.5.1, and suitable approaches for heterodimerization are described in Section 6.5.1.2. The Fc domains can include mutations that reduce effector function as compared to wild type Fc domains, e.g., as described in Section 6.5.1.1.

Exemplary spike protein ABD structures (e.g., CDR or VH/VL sequences) are disclosed in Section 6.3.

### 6.3. Spike Protein Antigen-Binding Domain

The present disclosure relates to multivalent anti-spike protein binding molecules comprising a plurality of spike protein antigen binding domains (ABDs).

In some embodiments, a multivalent anti-spike protein binding molecule of the disclosure comprises two or more spike protein ABDs.

In some embodiments, the multivalent anti-spike protein binding molecules of the disclosure are monospecific, e.g., bind the same epitope on spike protein. In some of these embodiments, the spike protein ABDs are identical.

In other embodiments, the multivalent anti-spike protein binding molecules of the disclosure are multispecific, e.g., bind to different epitopes. In some embodiments, the multispecific anti-spike protein binding molecules bind to different epitopes on the same spike protein. In other embodiments, multispecific anti-spike protein binding molecules bind to different epitopes on different spike protein variants. The different epitopes can correspond to sequence variants of the same region in a spike protein or in different regions altogether.

In further embodiments, a plurality or all of the ABDs in the multivalent anti-spike protein binding molecules of the disclosure bind to the receptor binding domain (RBD) of a spike protein and/or are capable of blocking or neutralizing spike protein, e.g., inhibit the ability of spike protein to bind to a receptor such as ACE2, to be cleaved by a protease such as TMPRSS2, or to mediate viral entry into a host cell or viral reproduction in a host cell.

Suitable spike protein ABD formats are described in Section 6.4. The spike protein ABD can be, for example, an antibody or an antigen-binding portion of an antibody, e.g., a Fab, as described in Section 6.4.1 or an scFv, as described in Section 6.4.2.

In some embodiments, a spike protein ABD competes with exemplary antibody or an antibody having the sequence set forth in Table 1 below for binding to spike protein and/or comprises binding portions of an exemplary antibody or an antibody having an antibody sequence set forth in Table 1. In some aspects, the spike protein ABD competes with an antibody set forth in Table 1 for binding to a spike protein. In further aspects, the spike protein ABD comprises CDRs having CDR sequences of an antibody set forth in Table 1. In some embodiments, the spike protein ABD comprises all 6 CDR sequences of the antibody set forth in Table 1. In other embodiments, the spike protein ABD comprises at least the heavy chain CDR sequences (CDR-H1, CDR-H2, CDR-H3 and the light chain CDR sequences of a universal light chain. In further aspects, a spike protein ABD comprises a VH comprising the amino acid sequence of the VH of an antibody set forth in Table 1. In some embodiments, the spike protein ABD further comprises a VL comprising the amino acid sequence of the VL of the antibody set forth in Table 1. In other embodiments, the spike protein ABD further comprises a universal light chain VL sequence.

TABLE 1
Exemplary Spike Protein Antibodies and Antigen-Binding Domains

| | | Blocking/Non- |
|---|---|---|
| | | blocking ACE2 |
| Target/Epitope | Antibody Name and/or Binding Sequences | binding |
| SARS-CoV-2 | P5-22 | Blocking |
| Spike protein | VH: SEQ ID NO: 2 of PCT Publication No: WO | |
| | 2022/052968 A1 | |
| | VL: SEQ ID NO: 4 of PCT Publication No: WO | |
| | 2022/052968 A1 | |
| SARS-CoV-2 | P14-44 | Blocking |
| Spike protein | VH: SEQ ID NO: 6 of PCT Publication No: WO | |
| | 2022/052968 A1 | |
| | VL: SEQ ID NO: 8 of PCT Publication No: WO | |
| | 2022/052968 A1 | |
| SARS-CoV-2 | P15-16 | Blocking |
| Spike protein | VH: SEQ ID NO: 10 of PCT Publication No: WO | |
| | 2022/052968 A1 | |
| | VL: SEQ ID NO: 12 of PCT Publication No: WO | |
| | 2022/052968 A1 | |
| SARS-CoV-2 | P10-20 | Blocking |
| Spike protein | VH: SEQ ID NO: 14 of PCT Publication No: WO | |
| | 2022/052968 A1 | |
| | VL: SEQ ID NO: 16 of PCT Publication No: WO | |
| | 2022/052968 A1 | |
| SARS-CoV-2 | P14-37 | Blocking |
| Spike protein | VH: SEQ ID NO: 18 of PCT Publication No: WO | |
| | 2022/052968 A1 | |
| | VL: SEQ ID NO: 20 of PCT Publication No: WO | |
| | 2022/052968 A1 | |
| SARS-CoV-2 | P23-29 | Blocking |
| Spike protein | VH: SEQ ID NO: 22 of PCT Publication No: WO | |
| | 2022/052968 A1 | |
| | VL: SEQ ID NO: 24 of PCT Publication No: WO | |
| | 2022/052968 A1 | |
| SARS-CoV-2 | P3-11 | Blocking |
| Spike protein | VH: SEQ ID NO: 26 of PCT Publication No: WO | |
| | 2022/052968 A1 | |
| | VL: SEQ ID NO: 28 of PCT Publication No: WO | |
| | 2022/052968 A1 | |
| aa 319-532 of S- | P16-A3 | Blocking |
| protein RBD | VH: SEQ ID NO: 17 of PCT Publication No: WO | |
| domain | 2021/196268 A1 | |
| | VL: SEQ ID NO: 18 of PCT Publication No: WO | |
| | 2021/196268 A1 | |
| aa 319-532 of S- | P17-A11 | Blocking |
| protein RBD | VH: SEQ ID NO: 19 of PCT Publication No: WO | |
| domain | 2021/196268 A1 | |
| | VL: SEQ ID NO: 20 of PCT Publication No: WO | |
| | 2021/196268 A1 | |
| aa 319-532 of S- | R15-F7 | Blocking |
| protein RBD | VH: SEQ ID NO: 1 of PCT Publication No: WO | |
| domain | 2022/104918 A1 | |
| | VL: SEQ ID NO: 2 of PCT Publication No: WO | |
| | 2022/104918 A1 | |
| S-protein RBD | DH1041 | Blocking |
| domain | VH: SEQ ID NO: 1100 of PCT Publication No: WO | |
| | 2022/060906 A1 | |
| | VL: SEQ ID NO: 1102 of PCT Publication No: WO | |
| | 2022/060906 A1 | |
| aa 481-491 of S- | DH1042 | Blocking |
| protein RBD | VH: SEQ ID NO: 1104 of PCT Publication No: WO | |
| domain | 2022/060906 A1 | |
| | VL: SEQ ID NO: 1106 of PCT Publication No: WO | |
| | 2022/060906 A1 | |
| S-protein RBD | DH1043 | Blocking |
| domain | VH: SEQ ID NO: 1108 of PCT Publication No: WO | |
| | 2022/060906 A1 | |
| | VL: SEQ ID NO: 1110 of PCT Publication No: WO | |
| | 2022/060906 A1 | |
| S-protein RBD | DH1044 | Non-blocking |
| domain | VH: SEQ ID NO: 1112 of PCT Publication No: WO | |
| | 2022/060906 A1 | |
| | VL: SEQ ID NO: 1114 of PCT Publication No: WO | |
| | 2022/060906 A1 | |
| S-protein RBD | DH1221 | Blocking |
| domain | VH: SEQ ID NO: 1214 of PCT Publication No: WO | |
| | 2022/060906 A1 | |
| | VL: SEQ ID NO: 1226 of PCT Publication No: WO | |
| | 2022/060906 A1 | |
| S-protein RBD | DH1222 | Blocking |
| domain | VH: SEQ ID NO: 1216 of PCT Publication No: WO | |
| | 2022/060906 A1 | |
| | VL: SEQ ID NO: 1228 of PCT Publication No: WO | |
| | 2022/060906 A1 | |
| S-protein RBD | DH1223 | Blocking |
| domain | VH: SEQ ID NO: 1218 of PCT Publication No: WO | |
| | 2022/060906 A1 | |
| | VL: SEQ ID NO: 1230 of PCT Publication No: WO | |
| | 2022/060906 A1 | |
| S-protein RBD | DH1224 | Blocking |
| domain | VH: SEQ ID NO: 1220 of PCT Publication No: WO | |
| | 2022/060906 A1 | |
| | VL: SEQ ID NO: 1232 of PCT Publication No: WO | |
| | 2022/060906 A1 | |
| S-protein RBD | DH1225 | Blocking |
| domain | VH: SEQ ID NO: 1222 of PCT Publication No: WO | |
| | 2022/060906 A1 | |
| | VL: SEQ ID NO: 1234 of PCT Publication No: WO | |
| | 2022/060906 A1 | |
| S-protein RBD | DH1226 | Blocking |
| domain | VH: SEQ ID NO: 1224 of PCT Publication No: WO | |
| | 2022/060906 A1 | |
| | VL: SEQ ID NO: 1236 of PCT Publication No: WO | |
| | 2022/060906 A1 | |
| S-protein RBD | mAb S1D2 | Blocking |
| domain | VH: SEQ ID NO: 6 of PCT Publication No: WO | |
| SEQ ID NO: 5 of | 2022/032139 A1 | |
| PCT WO | VL: SEQ ID NO: 7 of PCT Publication No: WO | |
| 2022/032139 A1 | 2022/032139 A1 | |
| S-protein RBD | mAb S1D7270 | Blocking |
| domain | VH: SEQ ID NO: 28 of PCT Publication No: WO | |
| SEQ ID NO: 5 of | 2022/032139 A1 | |
| PCT WO | VL: SEQ ID NO: 29 of PCT Publication No: WO | |
| 2022/032139 A1 | 2022/032139 A1 | |
| aa 331-524 of S- | 413-2 | |
| protein RBD | VH: SEQ ID NO: 15 of PCT Publication No: WO | |
| domain | 2021/248279 A1 | |
| SEQ ID NO: 61 | VL: SEQ ID NO: 43 of PCT Publication No: WO | |
| | 2021/248279 A1 | |
| aa 331-524 of S- | 413-3 | |
| protein RBD | VH: SEQ ID NO: 16 of PCT Publication No: WO | |
| domain | 2021/248279 A1 | |
| SEQ ID NO: 61 | VL: SEQ ID NO: 44 of PCT Publication No: WO | |
| | 2021/248279 A1 | |
| aa 331-524 of S- | 505-1 | |
| protein RBD | VH: SEQ ID NO: 17 of PCT Publication No: WO | |
| domain | 2021/248279 A1 | |
| SEQ ID NO: 61 | VL: SEQ ID NO: 45 of PCT Publication No: WO | |
| | 2021/248279 A1 | |
| aa 331-524 of S- | 505-3 | |
| protein RBD | VH: SEQ ID NO: 18 of PCT Publication No: WO | |
| domain | 2021/248279 A1 | |
| SEQ ID NO: 61 | VL: SEQ ID NO: 46 of PCT Publication No: WO | |
| | 2021/248279 A1 | |
| aa 331-524 of S- | 505-5 | |
| protein RBD | VH: SEQ ID NO: 19 of PCT Publication No: WO | |
| domain | 2021/248279 A1 | |
| SEQ ID NO: 61 | VL: SEQ ID NO: 47 of PCT Publication No: WO | |
| | 2021/248279 A1 | |
| aa 331-524 of S- | 515-5 | |
| protein RBD | VH: SEQ ID NO: 20 of PCT Publication No: WO | |
| domain | 2021/248279 A1 | |
| SEQ ID NO: 61 | VL: SEQ ID NO: 48 of PCT Publication No: WO | |
| | 2021/248279 A1 | |
| aa 331-524 of S- | 553-13 | |
| protein RBD | VH: SEQ ID NO: 21 of PCT Publication No: WO | |
| domain | 2021/248279 A1 | |
| SEQ ID NO: 61 | VL: SEQ ID NO: 49 of PCT Publication No: WO | |
| | 2021/248279 A1 | |
| aa 331-524 of S- | 553-15 | |
| protein RBD | VH: SEQ ID NO: 22 of PCT Publication No: WO | |
| domain | 2021/248279 A1 | |
| SEQ ID NO: 61 | VL: SEQ ID NO: 50 of PCT Publication No: WO | |
| | 2021/248279 A1 | |
| aa 331-524 of S- | 553-17 | |
| protein RBD | VH: SEQ ID NO: 23 of PCT Publication No: WO | |
| domain | 2021/248279 A1 | |
| SEQ ID NO: 61 | VL: SEQ ID NO: 51 of PCT Publication No: WO | |
| | 2021/248279 A1 | |
| aa 331-524 of S- | 553-18 | |
| protein RBD | VH: SEQ ID NO: 24 of PCT Publication No: WO | |
| domain | 2021/248279 A1 | |
| SEQ ID NO: 61 | VL: SEQ ID NO: 52 of PCT Publication No: WO | |
| | 2021/248279 A1 | |
| aa 331-524 of S- | 553-20 | |
| protein RBD | VH: SEQ ID NO: 25 of PCT Publication No: WO | |
| domain | 2021/248279 A1 | |
| SEQ ID NO: 61 | VL: SEQ ID NO: 53 of PCT Publication No: WO | |
| | 2021/248279 A1 | |
| aa 331-524 of S- | 553-27 | |
| protein RBD | VH: SEQ ID NO: 26 of PCT Publication No: WO | |
| domain | 2021/248279 A1 | |
| SEQ ID NO: 61 | VL: SEQ ID NO: 54 of PCT Publication No: WO | |
| | 2021/248279 A1 | |
| aa 331-524 of S- | 553-60 | |
| protein RBD | VH: SEQ ID NO: 27 of PCT Publication No: WO | |
| domain | 2021/248279 A1 | |
| SEQ ID NO: 61 | VL: SEQ ID NO: 55 of PCT Publication No: WO | |
| | 2021/248279 A1 | |
| aa 331-524 of S- | 553-63 | |
| protein RBD | VH: SEQ ID NO: 28 of PCT Publication No: WO | |
| domain | 2021/248279 A1 | |
| SEQ ID NO: 61 | VL: SEQ ID NO: 56 of PCT Publication No: WO | |
| | 2021/248279 A1 | |
| SARS-CoV-2 | N3-1 | |
| Spike protein | VH: SEQ ID NO: 1 of PCT Publication No: WO | |
| RBD | 2022/192661 A1 | |
| | VL: SEQ ID NO: 2 of PCT Publication No: WO | |
| | 2022/192661 A1 | |
| SARS-CoV-2 | N3-3 | |
| Spike protein | VH: SEQ ID NO: 1 of PCT Publication No: WO | |
| RBD | 2022/192661 A1 | |
| | VL: SEQ ID NO: 15 of PCT Publication No: WO | |
| | 2022/192661 A1 | |
| SARS-CoV-2 | 1D4 | |
| Spike protein | VH: SEQ ID NO: 18 of PCT Publication No: WO | |
| RBD | 2022/192661 A1 | |
| | VL: SEQ ID NO: 19 of PCT Publication No: WO | |
| | 2022/192661 A1 | |
| SARS-CoV-2 | N3-7 | |
| Spike protein | VH: SEQ ID NO: 1 of PCT Publication No: WO | |
| RBD | 2022/192661 A1 | |
| | VL: SEQ ID NO: 10 of PCT Publication No: WO | |
| | 2022/192661 A1 | |
| SARS-CoV-2 | N3-63A | |
| Spike protein | VH: SEQ ID NO: 1 of PCT Publication No: WO | |
| RBD | 2022/192661 A1 | |
| | VL: SEQ ID NO: 21 of PCT Publication No: WO | |
| | 2022/192661 A1 | |
| SARS-CoV-2 | N3-63B | |
| Spike protein | VH: SEQ ID NO: 1 of PCT Publication No: WO | |
| RBD | 2022/192661 A1 | |
| | VL: SEQ ID NO: 27 of PCT Publication No: WO | |
| | 2022/192661 A1 | |
| SARS-CoV-2 | N3-63C | |
| Spike protein | VH: SEQ ID NO: 1 of PCT Publication No: WO | |
| RBD | 2022/192661 A1 | |
| | VL: SEQ ID NO: 37 of PCT Publication No: WO | |
| | 2022/192661 A1 | |
| SARS-CoV-2 | 8B5 | |
| Spike protein | VH: SEQ ID NO: 22 of PCT Publication No: WO | |
| RBD | 2022/192661 A1 | |
| | VL: SEQ ID NO: 23 of PCT Publication No: WO | |
| | 2022/192661 A1 | |
| SARS-CoV-2 | 4A5 | |
| Spike protein | VH: SEQ ID NO: 24 of PCT Publication No: WO | |
| RBD | 2022/192661 A1 | |
| | VL: SEQ ID NO: 23 of PCT Publication No: WO | |
| | 2022/192661 A1 | |
| SARS-CoV-2 | 1D1 | |
| Spike protein | VH: SEQ ID NO: 18 of PCT Publication No: WO | |
| RBD | 2022/192661 A1 | |
| | VL: SEQ ID NO: 2 of PCT Publication No: WO | |
| | 2022/192661 A1 | |
| SARS-CoV-2 | 1D9 | |
| Spike protein | VH: SEQ ID NO: 18 of PCT Publication No: WO | |
| RBD | 2022/192661 A1 | |
| | VL: SEQ ID NO: 26 of PCT Publication No: WO | |
| | 2022/192661 A1 | |
| SARS-CoV-2 | 4A7 | |
| Spike protein | VH: SEQ ID NO: 24 of PCT Publication No: WO | |
| RBD | 2022/192661 A1 | |
| | VL: SEQ ID NO: 10 of PCT Publication No: WO | |
| | 2022/192661 A1 | |
| SARS-CoV-2 | 1D5 | |
| Spike protein | VH: SEQ ID NO: 18 of PCT Publication No: WO | |
| RBD | 2022/192661 A1 | |
| | VL: SEQ ID NO: 23 of PCT Publication No: WO | |
| | 2022/192661 A1 | |

In some embodiments, the spike protein ABDs comprise an amino acid sequence or are encoded by a nucleotide sequence set forth in Table 2 below. In particular aspects, the spike protein ABD comprises both heavy and light chain CDRs of an antibody set forth in Table 2 below. In other embodiments, the spike protein ABD comprises at least the heavy chain CDR sequences and the light chain CDR sequences of a universal light chain. In further aspects, a spike protein ABD comprises a VH having the amino acid sequence of the VH of an antibody set forth in Table 2 and a VL having the amino acid sequence of the VL of the same antibody as set forth in Table 2. In other aspects, a spike protein ABD comprises a VH having the amino acid sequence of the VH of an antibody set forth in Table 2 and a universal light chain VL sequence.

TABLE 2

| Antibody | Component | |
|---|---|---|
| Designation | Part | Sequence |
| mAb10933 | Amino Acids | |
| | VH | QVQLVESGGGLVKPGGSLRLSCAASGFTFSDYYMSWIRQAP |
| | | GKGLEWVSYITYSGSTIYYADSVKGRFTISRDNAKSSLYLQ |
| | | MNSLRAEDTAVYYCARDRGTTMVPFDYWGQGTLVTVSS |
| | | (SEQ ID NO: 1) |
| | CDR-H1 | GFTFSDYY (SEQ ID NO: 2) |
| | CDR-H2 | ITYSGSTI (SEQ ID NO: 3) |
| | CDR-H3 | ARDRGTTMVPFDY (SEQ ID NO: 4) |
| | VL | DIQMTQSPSSLSASVGDRVTITCQASQDITNYLNWYQQKPG |
| | | KAPKLLIYAASNLETGVPSRFSGSGSGTDFTFTISGLQPED |
| | | IATYYCQQYDNLPLTFGGGTKVEIK (SEQ ID NO: 5) |
| | CDR-L1 | QDITNY (SEQ ID NO: 6) |
| | CDR-L2 | AAS |
| | CDR-L3 | QQYDNLPLT (SEQ ID NO: 7) |
| | HC | QVQLVESGGGLVKPGGSLRLSCAASGFTFSDYYMSWIRQAP |
| | | GKGLEWVSYITYSGSTIYYADSVKGRFTISRDNAKSSLYLQ |
| | | MNSLRAEDTAVYYCARDRGTTMVPFDYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 8) |
| | LC | DIQMTQSPSSLSASVGDRVTITCQASQDITNYLNWYQQKPG |
| | | KAPKLLIYAASNLETGVPSRFSGSGSGTDFTFTISGLQPED |
| | | IATYYCQQYDNLPLTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 9) |
| | VH | CAGGTGCAGCTGGTGGAGTCTGGGGGAGGCTTGGTCAAGCC |
| | | TGGAGGGTCCCTGAGACTCTCCTGTGCAGCCTCTGGATTCA |
| | | CCTTCAGTGACTACTACATGAGCTGGATCCGCCAGGCTCCA |
| | | GGGAAGGGGCTGGAGTGGGTTTCATACATTACTTATAGTGG |
| | | TAGTACCATATACTACGCAGACTCTGTGAAGGGCCGATTCA |
| | | CCATCTCCAGGGACAACGCCAAGAGCTCACTGTATCTGCAA |
| | | ATGAACAGCCTGAGAGCCGAGGACACGGCCGTGTATTACTG |
| | | TGCGAGAGATCGCGGTACAACTATGGTCCCCTTTGACTACT |
| | | GGGGCCAGGGAACCCTGGTCACCGTCTCCTCA (SEQ ID |
| | | NO: 10) |
| | CDR-H1 | GGATTCACCTTCAGTGACTACTAC (SEQ ID NO: 11) |
| | CDR-H2 | ATTACTTATAGTGGTAGTACCATA (SEQ ID NO: 12) |
| | CDR-H3 | GCGAGAGATCGCGGTACAACTATGGTCCCCTTTGACTAC |
| | | (SEQ ID NO: 13) |
| | VL | GACATCCAGATGACCCAGTCTCCATCCTCCCTGTCTGCATC |
| | | TGTAGGAGACAGAGTCACCATCACTTGCCAGGCGAGTCAGG |
| | | ACATTACCAACTATTTAAATTGGTATCAGCAGAAACCAGGG |
| | | AAAGCCCCTAAGCTCCTGATCTACGCTGCATCCAATTTGGA |
| | | AACAGGGGTCCCATCAAGGTTCAGTGGAAGTGGATCTGGGA |
| | | CAGATTTTACTTTCACCATCAGCGGCCTGCAGCCTGAAGAT |
| | | ATTGCAACATATTACTGTCAACAGTATGATAATCTCCCTCT |
| | | CACTTTCGGCGGAGGGACCAAGGTGGAGATCAAA (SEQ |
| | | ID NO: 14) |
| | CDR-L1 | CAGGACATTACCAACTAT (SEQ ID NO: 15) |
| | CDR-L2 | GCTGCATCC |
| | CDR-L3 | CAACAGTATGATAATCTCCCTCTCACT (SEQ ID |
| | | NO: 16) |
| | HC | CAGGTGCAGCTGGTGGAGTCTGGGGGAGGCTTGGTCAAGCC |
| | | TGGAGGGTCCCTGAGACTCTCCTGTGCAGCCTCTGGATTCA |
| | | CCTTCAGTGACTACTACATGAGCTGGATCCGCCAGGCTCCA |
| | | GGGAAGGGGCTGGAGTGGGTTTCATACATTACTTATAGTGG |
| | | TAGTACCATATACTACGCAGACTCTGTGAAGGGCCGATTCA |
| | | CCATCTCCAGGGACAACGCCAAGAGCTCACTGTATCTGCAA |
| | | ATGAACAGCCTGAGAGCCGAGGACACGGCCGTGTATTACTG |
| | | TGCGAGAGATCGCGGTACAACTATGGTCCCCTTTGACTACT |
| | | GGGGCCAGGGAACCCTGGTCACCGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCC |
| | | AAAGCCAAAGGGCAGCCCCGAGAACCACAGGTGTACACCCT |
| | | GCCCCCATCCCGGGATGAGCTGACCAAGAACCAGGTCAGCC |
| | | TGACCTGCCTGGTCAAAGGCTTCTATCCCAGCGACATCGCC |
| | | GTGGAGTGGGAGAGCAATGGGCAGCCGGAGAACAACTACAA |
| | | GACCACGCCTCCCGTGCTGGACTCCGACGGCTCCTTCTTCC |
| | | TCTACAGCAAGCTCACCGTGGACAAGAGCAGGTGGCAGCAG |
| | | GGGAACGTCTTCTCATGCTCCGTGATGCATGAGGCTCTGCA |
| | | CAACCACTACACGCAGAAGTCCCTCTCCCTGTCTCCGGGTA |
| | | AATGA (SEQ ID NO: 17) |
| | LC | GACATCCAGATGACCCAGTCTCCATCCTCCCTGTCTGCATC |
| | | TGTAGGAGACAGAGTCACCATCACTTGCCAGGCGAGTCAGG |
| | | ACATTACCAACTATTTAAATTGGTATCAGCAGAAACCAGGG |
| | | AAAGCCCCTAAGCTCCTGATCTACGCTGCATCCAATTTGGA |
| | | AACAGGGGTCCCATCAAGGTTCAGTGGAAGTGGATCTGGGA |
| | | CAGATTTTACTTTCACCATCAGCGGCCTGCAGCCTGAAGAT |
| | | ATTGCAACATATTACTGTCAACAGTATGATAATCTCCCTCT |
| | | CACTTTCGGCGGAGGGACCAAGGTGGAGATCAAACGAACTG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG (SEQ ID |
| | | NO: 19) |
| mAb10934 | VH | EVQLVESGGGLVKPGGSLRLSCAASGITFSNAWMSWVRQAP |
| | | GKGLEWVGRIKSKTDGGTTDYAAPVKGRFTISRDDSKNTLY |
| | | LQMNSLKTEDTAVYYCTTARWDWYFDLWGRGTLVTVSS |
| | | (SEQ ID NO: 20) |
| | CDR-H1 | GITFSNAW (SEQ ID NO: 21) |
| | CDR-H2 | IKSKTDGGTT (SEQ ID NO: 22) |
| | CDR-H3 | TTARWDWYFDL (SEQ ID NO: 23) |
| | VL | DIQMTQSPSSLSASVGDRVTITCQASQDIWNYINWYQQKPG |
| | | KAPKLLIYDASNLKTGVPSRFSGSGSGTDFTFTISSLQPED |
| | | IATYYCQQHDDLPPTFGQGTKVEIK (SEQ ID NO: 24) |
| | CDR-L1 | QDIWNY (SEQ ID NO: 25) |
| | CDR-L2 | DAS |
| | CDR-L3 | QQHDDLPPT (SEQ ID NO: 26) |
| | HC | EVQLVESGGGLVKPGGSLRLSCAASGITFSNAWMSWVRQAP |
| | | GKGLEWVGRIKSKTDGGTTDYAAPVKGRFTISRDDSKNTLY |
| | | LQMNSLKTEDTAVYYCTTARWDWYFDLWGRGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 27) |
| | LC | DIQMTQSPSSLSASVGDRVTITCQASQDIWNYINWYQQKPG |
| | | KAPKLLIYDASNLKTGVPSRFSGSGSGTDFTFTISSLQPED |
| | | IATYYCQQHDDLPPTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC (SEQ ID NO: 28) |
| | VH | GAGGTGCAGCTGGTGGAGTCTGGGGGAGGCTTGGTAAAGCC |
| | | TGGGGGGTCCCTTAGACTCTCCTGTGCAGCCTCTGGAATCA |
| | | CTTTCAGTAACGCCTGGATGAGTTGGGTCCGCCAGGCTCCA |
| | | GGGAAGGGGCTGGAGTGGGTTGGCCGTATTAAAAGCAAAAC |
| | | TGATGGTGGGACAACAGACTACGCCGCACCCGTGAAAGGCA |
| | | GATTCACCATCTCAAGAGATGATTCAAAAAACACGCTGTAT |
| | | CTACAAATGAACAGCCTGAAAACCGAGGACACAGCCGTGTA |
| | | TTACTGTACCACAGCGAGGTGGGACTGGTACTTCGATCTCT |
| | | GGGGCCGTGGCACCCTGGTCACTGTCTCCTCA (SEQ ID |
| | | NO: 29) |
| | CDR-H1 | GGAATCACTTTCAGTAACGCCTGG (SEQ ID NO: 30) |
| | CDR-H2 | ATTAAAAGCAAAACTGATGGTGGGACAACA (SEQ ID |
| | | NO: 31) |
| | CDR-H3 | ACCACAGCGAGGTGGGACTGGTACTTCGATCTC (SEQ ID |
| | | NO: 32) |
| | VL | GACATCCAGATGACCCAGTCTCCATCCTCCCTGTCTGCATC |
| | | TGTAGGAGACAGAGTCACCATCACTTGCCAGGCGAGTCAGG |
| | | ACATTTGGAATTATATAAATTGGTATCAGCAGAAACCAGGG |
| | | AAGGCCCCTAAGCTCCTGATCTACGATGCATCCAATTTGAA |
| | | AACAGGGGTCCCATCAAGGTTCAGTGGAAGTGGATCTGGGA |
| | | CAGATTTTACTTTCACCATCAGCAGCCTGCAGCCTGAAGAT |
| | | ATTGCAACATATTACTGTCAACAGCATGATGATCTCCCTCC |
| | | GACCTTCGGCCAAGGGACCAAGGTGGAAATCAAA (SEQ |
| | | ID NO: 33) |
| | CDR-L1 | CAGGACATTTGGAATTAT (SEQ ID NO: 34) |
| | CDR-L2 | GATGCATCC |
| | CDR-L3 | CAACAGCATGATGATCTCCCTCCGACC (SEQ ID |
| | | NO: 35) |
| | HC | GAGGTGCAGCTGGTGGAGTCTGGGGGAGGCTTGGTAAAGCC |
| | | TGGGGGGTCCCTTAGACTCTCCTGTGCAGCCTCTGGAATCA |
| | | CTTTCAGTAACGCCTGGATGAGTTGGGTCCGCCAGGCTCCA |
| | | GGGAAGGGGCTGGAGTGGGTTGGCCGTATTAAAAGCAAAAC |
| | | TGATGGTGGGACAACAGACTACGCCGCACCCGTGAAAGGCA |
| | | GATTCACCATCTCAAGAGATGATTCAAAAAACACGCTGTAT |
| | | CTACAAATGAACAGCCTGAAAACCGAGGACACAGCCGTGTA |
| | | TTACTGTACCACAGCGAGGTGGGACTGGTACTTCGATCTCT |
| | | GGGGCCGTGGCACCCTGGTCACTGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCC |
| | | AAAGCCAAAGGGCAGCCCCGAGAACCACAGGTGTACACCCT |
| | | GCCCCCATCCCGGGATGAGCTGACCAAGAACCAGGTCAGCC |
| | | TGACCTGCCTGGTCAAAGGCTTCTATCCCAGCGACATCGCC |
| | | GTGGAGTGGGAGAGCAATGGGCAGCCGGAGAACAACTACAA |
| | | GACCACGCCTCCCGTGCTGGACTCCGACGGCTCCTTCTTCC |
| | | TCTACAGCAAGCTCACCGTGGACAAGAGCAGGTGGCAGCAG |
| | | GGGAACGTCTTCTCATGCTCCGTGATGCATGAGGCTCTGCA |
| | | CAACCACTACACGCAGAAGTCCCTCTCCCTGTCTCCGGGTA |
| | | AATGA (SEQ ID NO: 36) |
| | LC | GACATCCAGATGACCCAGTCTCCATCCTCCCTGTCTGCATC |
| | | TGTAGGAGACAGAGTCACCATCACTTGCCAGGCGAGTCAGG |
| | | ACATTTGGAATTATATAAATTGGTATCAGCAGAAACCAGGG |
| | | AAGGCCCCTAAGCTCCTGATCTACGATGCATCCAATTTGAA |
| | | AACAGGGGTCCCATCAAGGTTCAGTGGAAGTGGATCTGGGA |
| | | CAGATTTTACTTTCACCATCAGCAGCCTGCAGCCTGAAGAT |
| | | ATTGCAACATATTACTGTCAACAGCATGATGATCTCCCTCC |
| | | GACCTTCGGCCAAGGGACCAAGGTGGAAATCAAACGAACTG |
| | | TGGCTGCACCATCTGTCTTCATCTTCCCGCCATCTGATGAG |
| | | CAGTTGAAATCTGGAACTGCCTCTGTTGTGTGCCTGCTGAA |
| | | TAACTTCTATCCCAGAGAGGCCAAAGTACAGTGGAAGGTGG |
| | | ATAACGCCCTCCAATCGGGTAACTCCCAGGAGAGTGTCACA |
| | | GAGCAGGACAGCAAGGACAGCACCTACAGCCTCAGCAGCAC |
| | | CCTGACGCTGAGCAAAGCAGACTACGAGAAACACAAAGTCT |
| | | ACGCCTGCGAAGTCACCCATCAGGGCCTGAGCTCGCCCGTC |
| | | ACAAAGAGCTTCAACAGGGGAGAGTGTTAG (SEQ ID |
| | | NO: 37) |
| mAb10987 | VH | QVQLVESGGGVVQPGRSLRLSCAASGFTFSNYAMYWVRQAP |
| | | GKGLEWVAVISYDGSNKYYADSVKGRFTISRDNSKNTLYLQ |
| | | MNSLRTEDTAVYYCASGSDYGDYLLVYWGQGTLVTVSS |
| | | (SEQ ID NO: 38) |
| | CDR-H1 | GFTFSNYA (SEQ ID NO: 39) |
| | CDR-H2 | ISYDGSNK (SEQ ID NO: 40) |
| | CDR-H3 | ASGSDYGDYLLVY (SEQ ID NO: 41) |
| | VL | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVFGGGTKLTVL (SEQ ID |
| | | NO: 42) |
| | CDR-L1 | SSDVGGYNY (SEQ ID NO: 43) |
| | CDR-L2 | DVS |
| | CDR-L3 | NSLTSISTWV (SEQ ID NO: 44) |
| | HC | QVQLVESGGGVVQPGRSLRLSCAASGFTFSNYAMYWVRQAP |
| | | GKGLEWVAVISYDGSNKYYADSVKGRFTISRDNSKNTLYLQ |
| | | MNSLRTEDTAVYYCASGSDYGDYLLVYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| | | (SEQ ID NO: 45) |
| | LC | QSALTQPASVSGSPGQSITISCTGTSSDVGGYNYVSWYQQH |
| | | PGKAPKLMIYDVSKRPSGVSNRFSGSKSGNTASLTISGLQS |
| | | EDEADYYCNSLTSISTWVFGGGTKLTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 46) |
| | VH | CAGGTGCAGCTGGTGGAGTCTGGGGGAGGCGTGGTCCAGCC |
| | | TGGGAGGTCCCTGAGACTCTCCTGTGCAGCCTCTGGATTCA |
| | | CCTTCAGTAACTATGCTATGTACTGGGTCCGCCAGGCTCCA |
| | | GGCAAGGGGCTGGAGTGGGTGGCAGTTATATCATATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAACTGAGGACACGGCTGTGTATTACTG |
| | | TGCGAGTGGCTCCGACTACGGTGACTACTTATTGGTTTACT |
| | | GGGGCCAGGGAACCCTGGTCACCGTCTCCTCA (SEQ ID |
| | | NO: 47) |
| | CDR-H1 | GGATTCACCTTCAGTAACTATGCT (SEQ ID NO: 48) |
| | CDR-H2 | ATATCATATGATGGAAGTAATAAA (SEQ ID NO: 409) |
| | CDR-H3 | GCGAGTGGCTCCGACTACGGTGACTACTTATTGGTTTAC |
| | | (SEQ ID NO: 50) |
| | VL | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTGGTTATAACTATGTCTCCTGGTACCAACAACAC |
| | | CCAGGCAAAGCCCCCAAACTCATGATTTATGATGTCAGTAA |
| | | GCGGCCCTCAGGGGTTTCTAATCGCTTCTCTGGCTCCAAGT |
| | | CTGGCAACACGGCCTCCCTGACCATCTCTGGGCTCCAGTCT |
| | | GAGGACGAGGCTGATTATTACTGCAACTCTTTGACAAGCAT |
| | | CAGCACTTGGGTGTTCGGCGGAGGGACCAAGCTGACCGTCC |
| | | TA (SEQ ID NO: 51) |
| | CDR-L1 | AGCAGTGACGTTGGTGGTTATAACTAT (SEQ ID |
| | | NO: 52) |
| | CDR-L2 | GATGTCAGT |
| | CDR-L3 | AACTCTTTGACAAGCATCAGCACTTGGGTG (SEQ ID |
| | | NO: 53) |
| | HC | CAGGTGCAGCTGGTGGAGTCTGGGGGAGGCGTGGTCCAGCC |
| | | TGGGAGGTCCCTGAGACTCTCCTGTGCAGCCTCTGGATTCA |
| | | CCTTCAGTAACTATGCTATGTACTGGGTCCGCCAGGCTCCA |
| | | GGCAAGGGGCTGGAGTGGGTGGCAGTTATATCATATGATGG |
| | | AAGTAATAAATACTATGCAGACTCCGTGAAGGGCCGATTCA |
| | | CCATCTCCAGAGACAATTCCAAGAACACGCTGTATCTGCAA |
| | | ATGAACAGCCTGAGAACTGAGGACACGGCTGTGTATTACTG |
| | | TGCGAGTGGCTCCGACTACGGTGACTACTTATTGGTTTACT |
| | | GGGGCCAGGGAACCCTGGTCACCGTCTCCTCAGCCTCCACC |
| | | AAGGGCCCATCGGTCTTCCCCCTGGCACCCTCCTCCAAGAG |
| | | CACCTCTGGGGGCACAGCGGCCCTGGGCTGCCTGGTCAAGG |
| | | ACTACTTCCCCGAACCGGTGACGGTGTCGTGGAACTCAGGC |
| | | GCCCTGACCAGCGGCGTGCACACCTTCCCGGCTGTCCTACA |
| | | GTCCTCAGGACTCTACTCCCTCAGCAGCGTGGTGACCGTGC |
| | | CCTCCAGCAGCTTGGGCACCCAGACCTACATCTGCAACGTG |
| | | AATCACAAGCCCAGCAACACCAAGGTGGACAAGAAAGTTGA |
| | | GCCCAAATCTTGTGACAAAACTCACACATGCCCACCGTGCC |
| | | CAGCACCTGAACTCCTGGGGGGACCGTCAGTCTTCCTCTTC |
| | | CCCCCAAAACCCAAGGACACCCTCATGATCTCCCGGACCCC |
| | | TGAGGTCACATGCGTGGTGGTGGACGTGAGCCACGAAGACC |
| | | CTGAGGTCAAGTTCAACTGGTACGTGGACGGCGTGGAGGTG |
| | | CATAATGCCAAGACAAAGCCGCGGGAGGAGCAGTACAACAG |
| | | CACGTACCGTGTGGTCAGCGTCCTCACCGTCCTGCACCAGG |
| | | ACTGGCTGAATGGCAAGGAGTACAAGTGCAAGGTCTCCAAC |
| | | AAAGCCCTCCCAGCCCCCATCGAGAAAACCATCTCC |
| | | AAAGCCAAAGGGCAGCCCCGAGAACCACAGGTGTACACCCT |
| | | GCCCCCATCCCGGGATGAGCTGACCAAGAACCAGGTCAGCC |
| | | TGACCTGCCTGGTCAAAGGCTTCTATCCCAGCGACATCGCC |
| | | GTGGAGTGGGAGAGCAATGGGCAGCCGGAGAACAACTACAA |
| | | GACCACGCCTCCCGTGCTGGACTCCGACGGCTCCTTCTTCC |
| | | TCTACAGCAAGCTCACCGTGGACAAGAGCAGGTGGCAGCAG |
| | | GGGAACGTCTTCTCATGCTCCGTGATGCATGAGGCTCTGCA |
| | | CAACCACTACACGCAGAAGTCCCTCTCCCTGTCTCCGGGTA |
| | | AATGA (SEQ ID NO: 54) |
| | LC | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTGGTTATAACTATGTCTCCTGGTACCAACAACAC |
| | | CCAGGCAAAGCCCCCAAACTCATGATTTATGATGTCAGTAA |
| | | GCGGCCCTCAGGGGTTTCTAATCGCTTCTCTGGCTCCAAGT |
| | | CTGGCAACACGGCCTCCCTGACCATCTCTGGGCTCCAGTCT |
| | | GAGGACGAGGCTGATTATTACTGCAACTCTTTGACAAGCAT |
| | | CAGCACTTGGGTGTTCGGCGGAGGGACCAAGCTGACCGTCC |
| | | TAGGCCAGCCCAAGGCCGCCCCCTCCGTGACCCTGTTCCCC |
| | | CCCTCCTCCGAGGAGCTGCAGGCCAACAAGGCCACCCTGGT |
| | | GTGCCTGATCTCCGACTTCTACCCCGGCGCCGTGACCGTGG |
| | | CCTGGAAGGCCGACTCCTCCCCCGTGAAGGCCGGCGTGGAG |
| | | ACCACCACCCCCTCCAAGCAGTCCAACAACAAGTACGCCGC |
| | | CTCCTCCTACCTGTCCCTGACCCCCGAGCAGTGGAAGTCCC |
| | | ACCGGTCCTACTCCTGCCAGGTGACCCACGAGGGCTCCACC |
| | | GTGGAGAAGACCGTGGCCCCCACCGAGTGCTCCTGA (SEQ |
| | | ID NO: 55) |
| mAb10989 | VH | QVQLVQSGAEVKKPGASVKVSCKASGYIFTGYYMHWVRQAP |
| | | GQGLEWMGWINPNSGGANYAQKFQGRVTLTRDTSITTVYME |
| | | LSRLREDDTAVYYCARGSRYDWNQNNWEDPWGQGTLVTVSS |
| | | (SEQ ID NO: 56) |
| | CDR-H1 | GYIFTGYY (SEQ ID NO: 57) |
| | CDR-H2 | INPNSGGA (SEQ ID NO: 58) |
| | CDR-H3 | ARGSRYDWNQNNWFDP (SEQ ID NO: 59) |
| | VL | QSALTQPASVSGSPGQSITISCTGTSSDVGTYNYVSWYQQH |
| | | PGKAPKLMIFDVSNRPSGVSDRFSGSKSGNTASLTISGLQA |
| | | EDEADYYCSSFTTSSTVVFGGGTKLTVL (SEQ ID |
| | | NO: 60) |
| | CDR-L1 | SSDVGTYNY (SEQ ID NO: 61) |
| | CDR-L2 | DVS |
| | CDR-L3 | SSFTTSSTVV (SEQ ID NO: 62) |
| | HC | QVQLVQSGAEVKKPGASVKVSCKASGYIFTGYYMHWVRQAP |
| | | GQGLEWMGWINPNSGGANYAQKFQGRVTLTRDTSITTVYME |
| | | LSRLREDDTAVYYCARGSRYDWNQNNWEDPWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYI |
| | | CNVNHKPSNTKVDKKVEPKSCDKTHTCPPCPAPELLGGPSV |
| | | FLFPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQV |
| | | SLTCLVKGFYPSDIAVEWESNGQPENNYKTTPPVLDSDGSF |
| | | FLYSKLTVDKSRWQQGNVFSCSVMHEALHNHYTQKSLSLSP |
| | | GK (SEQ ID NO: 63) |
| | LC | QSALTQPASVSGSPGQSITISCTGTSSDVGTYNYVSWYQQH |
| | | PGKAPKLMIFDVSNRPSGVSDRFSGSKSGNTASLTISGLQA |
| | | EDEADYYCSSFTTSSTVVFGGGTKLTVLGQPKAAPSVTLEP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS (SEQ ID NO: 64) |
| | VH | CAGGTGCAGCTGGTGCAGTCTGGGGCTGAGGTGAAGAAGCC |
| | | TGGGGCCTCAGTGAAGGTCTCCTGCAAGGCTTCTGGATACA |
| | | TCTTCACCGGCTACTATATGCACTGGGTGCGACAGGCCCCT |
| | | GGACAGGGGCTTGAGTGGATGGGATGGATCAACCCTAACAG |
| | | TGGTGGCGCAAACTATGCACAGAAGTTTCAGGGCAGGGTCA |
| | | CCCTGACCAGGGACACGTCCATCACCACAGTCTACATGGAA |
| | | CTGAGCAGGCTGAGATTTGACGACACGGCCGTGTATTACTG |
| | | TGCGAGAGGATCCCGGTATGACTGGAACCAGAACAACTGGT |
| | | TCGACCCCTGGGGCCAGGGAACCCTGGTCACCGTCTCCTCA |
| | | (SEQ ID NO: 65) |
| | CDR-H1 | GGATACATCTTCACCGGCTACTAT (SEQ ID NO: 66) |
| | CDR-H2 | ATCAACCCTAACAGTGGTGGCGCA (SEQ ID NO: 67) |
| | CDR-H3 | GCGAGAGGATCCCGGTATGACTGGAACCAGAACAACTGGTT |
| | | CGACCCC (SEQ ID NO: 68) |
| | VL | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTACTTATAACTATGTCTCCTGGTACCAACAACAC |
| | | CCAGGCAAAGCCCCCAAACTCATGATTTTTGATGTCAGTAA |
| | | TCGGCCCTCAGGGGTTTCTGATCGCTTCTCTGGCTCCAAGT |
| | | CTGGCAACACGGCCTCCCTGACCATCTCTGGGCTCCAGGCT |
| | | GAGGACGAGGCTGATTATTACTGCAGCTCATTTACAACCAG |
| | | CAGCACTGTGGTTTTCGGCGGAGGGACCAAGCTGACCGTCC |
| | | TA (SEQ ID NO: 69) |
| | CDR-L1 | AGCAGTGACGTTGGTACTTATAACTAT (SEQ ID |
| | | NO: 70) |
| | CDR-L2 | GATGTCAGT |
| | CDR-L3 | AGCTCATTTACAACCAGCAGCACTGTGGTT (SEQ ID |
| | | NO: 71) |
| | HC | CAGGTGCAGCTGGTGCAGTCTGGGGCTGAGGTGAAGAAGCC |
| | | TGGGGCCTCAGTGAAGGTCTCCTGCAAGGCTTCTGGATACA |
| | | TCTTCACCGGCTACTATATGCACTGGGTGCGACAGGCCCCT |
| | | GGACAGGGGCTTGAGTGGATGGGATGGATCAACCCTAACAG |
| | | TGGTGGCGCAAACTATGCACAGAAGTTTCAGGGCAGGGTCA |
| | | CCCTGACCAGGGACACGTCCATCACCACAGTCTACATGGAA |
| | | CTGAGCAGGCTGAGATTTGACGACACGGCCGTGTATTACTG |
| | | TGCGAGAGGATCCCGGTATGACTGGAACCAGAACAACTGGT |
| | | TCGACCCCTGGGGCCAGGGAACCCTGGTCACCGTCTCCTCA |
| | | GCCTCCACCAAGGGCCCATCGGTCTTCCCCCTGGCACCCTC |
| | | CTCCAAGAGCACCTCTGGGGGCACAGCGGCCCTGGGCTGCC |
| | | TGGTCAAGGACTACTTCCCCGAACCGGTGACGGTGTCGTGG |
| | | AACTCAGGCGCCCTGACCAGCGGCGTGCACACCTTCCCGGC |
| | | TGTCCTACAGTCCTCAGGACTCTACTCCCTCAGCAGCGTGG |
| | | TGACCGTGCCCTCCAGCAGCTTGGGCACCCAGACCTACATC |
| | | TGCAACGTGAATCACAAGCCCAGCAACACCAAGGTGGACAA |
| | | GAAAGTTGAGCCCAAATCTTGTGACAAAACTCACACATGCC |
| | | CACCGTGCCCAGCACCTGAACTCCTGGGGGGACCGTCAGTC |
| | | TTCCTCTTCCCCCCAAAACCCAAGGACACCCTCATGATCTC |
| | | CCGGACCCCTGAGGTCACATGCGTGGTGGTGGACGTGAGCC |
| | | ACGAAGACCCTGAGGTCAAGTTCAACTGGTACGTGGACGGC |
| | | GTGGAGGTGCATAATGCCAAGACAAAGCCGCGGGAGGAGCA |
| | | GTACAACAGCACGTACCGTGTGGTCAGCGTCCTCACCGTCC |
| | | TGCACCAGGACTGGCTGAATGGCAAGGAGTACAAGTGCAAG |
| | | GTCTCCAACAAAGCCCTCCCAGCCCCCATCGAGAAA |
| | | ACCATCTCCAAAGCCAAAGGGCAGCCCCGAGAACCACAGGT |
| | | GTACACCCTGCCCCCATCCCGGGATGAGCTGACCAAGAACC |
| | | AGGTCAGCCTGACCTGCCTGGTCAAAGGCTTCTATCCCAGC |
| | | GACATCGCCGTGGAGTGGGAGAGCAATGGGCAGCCGGAGAA |
| | | CAACTACAAGACCACGCCTCCCGTGCTGGACTCCGACGGCT |
| | | CCTTCTTCCTCTACAGCAAGCTCACCGTGGACAAGAGCAGG |
| | | TGGCAGCAGGGGAACGTCTTCTCATGCTCCGTGATGCATGA |
| | | GGCTCTGCACAACCACTACACGCAGAAGTCCCTCTCCCTGT |
| | | CTCCGGGTAAATGA (SEQ ID NO: 72) |
| | LC | CAGTCTGCCCTGACTCAGCCTGCCTCCGTGTCTGGGTCTCC |
| | | TGGACAGTCGATCACCATCTCCTGCACTGGAACCAGCAGTG |
| | | ACGTTGGTACTTATAACTATGTCTCCTGGTACCAACAACAC |
| | | CCAGGCAAAGCCCCCAAACTCATGATTTTTGATGTCAGTAA |
| | | TCGGCCCTCAGGGGTTTCTGATCGCTTCTCTGGCTCCAAGT |
| | | CTGGCAACACGGCCTCCCTGACCATCTCTGGGCTCCAGGCT |
| | | GAGGACGAGGCTGATTATTACTGCAGCTCATTTACAACCAG |
| | | CAGCACTGTGGTTTTCGGCGGAGGGACCAAGCTGACCGTCC |
| | | TAGGCCAGCCCAAGGCCGCCCCCTCCGTGACCCTGTTCCCC |
| | | CCCTCCTCCGAGGAGCTGCAGGCCAACAAGGCCACCCTGGT |
| | | GTGCCTGATCTCCGACTTCTACCCCGGCGCCGTGACCGTGG |
| | | CCTGGAAGGCCGACTCCTCCCCCGTGAAGGCCGGCGTGGAG |
| | | ACCACCACCCCCTCCAAGCAGTCCAACAACAAGTACGCCGC |
| | | CTCCTCCTACCTGTCCCTGACCCCCGAGCAGTGGAAGTCCC |
| | | ACCGGTCCTACTCCTGCCAGGTGACCCACGAGGGCTCCACC |
| | | GTGGAGAAGACCGTGGCCCCCACCGAGTGCTCCTGA (SEQ |
| | | ID NO: 73) |

In further embodiments, the spike protein ABDs comprise an amino acid sequence set forth in Table 3 below. In particular aspects, the spike protein ABD comprises both heavy and light chain CDRs of an antibody set forth in Table 3 below. In other embodiments, the spike protein ABD comprises at least the heavy chain CDR sequences and the light chain CDR sequences of a universal light chain. In further aspects, a spike protein ABD comprises a VH having the amino acid sequence of the VH of an antibody set forth in Table 3 and a VL having the amino acid sequence of the VL of the same antibody as set forth in Table 3. In other aspects, a spike protein ABD comprises a VH having the amino acid sequence of the VH of an antibody set forth in Table 3 and a universal light chain VL sequence. The initial sequence identifiers for Table 3 are in relation to the sequence listing in WO 2021/045836A1, which sequence identifiers are incorporated by reference herein, while sequence identifiers presented parenthetically are those of the disclosure.

TABLE 3
SEQ ID NO in WO2021/045836A1 (SEQ ID NO of the disclosure)

| Antibody | | CDR- | CDR- | CDR- | | CDR- | CDR- | CDR- |
|---|---|---|---|---|---|---|---|---|
| Designation | VH | H1 | H2 | H3 | VL | L1 | L2 | L3 |
| mAb10913 | 2 | 4 | 6 | 8 | 10 | 12 | 14 | 16 |
| | (88) | (89) | (90) | (91) | (92) | (93) | (94) | (95) |
| mAb10915 | 22 | 24 | 26 | 28 | 30 | 32 | 34 | 36 |
| | (96) | (97) | (98) | (99) | (100) | (101) | (102) | (103) |
| mAb10916 | 2 | 4 | 6 | 8 | 10 | 12 | 14 | 16 |
| | (88) | (89) | (90) | (91) | (92) | (93) | (94) | (95) |
| mAb10917 | 44 | 46 | 26 | 49 | 51 | 53 | 55 | 57 |
| | (104) | (105) | (98) | (106) | (107) | (108) | (109) | (110) |
| mAb10918 | 22 | 24 | 26 | 28 | 30 | 32 | 34 | 36 |
| | (96) | (97) | (98) | (99) | (100) | (101) | (102) | (103) |
| mAb10920 | 65 | 67 | 69 | 71 | 73 | 75 | 55 | 77 |
| | (111) | (112) | (113) | (114) | (115) | (116) | (109) | (117) |
| mAb10921 | 83 | 85 | 26 | 87 | 89 | 91 | 55 | 93 |
| | (118) | (119) | (98) | (120) | (121) | (122) | (109) | (123) |
| mAb10922 | 99 | 101 | 103 | 105 | 107 | 109 | 111 | 113 |
| | (124) | (125) | (126) | (127) | (128) | (129) | (130) | (131) |
| mAb10923 | 119 | 121 | 123 | 125 | 127 | 129 | 55 | 131 |
| | (132) | (133) | (134) | (135) | (136) | (137) | (109) | (138) |
| mAb10924 | 137 | 139 | 141 | 143 | 145 | 147 | 149 | 151 |
| | (139) | (140) | (141) | (142) | (143) | (144) | (145) | (146) |
| mAb10925 | 65 | 67 | 69 | 71 | 73 | 75 | 55 | 77 |
| | (111) | (112) | (113) | (114) | (115) | (116) | (109) | (117) |
| mAb10926 | 83 | 85 | 26 | 87 | 89 | 91 | 55 | 93 |
| | (118) | (119) | (98) | (120) | (121) | (122) | (109) | (123) |
| mAb10927 | 99 | 101 | 103 | 105 | 107 | 109 | 111 | 113 |
| | (124) | (125) | (126) | (127) | (128) | (129) | (130) | (131) |
| mAb10928 | 119 | 121 | 123 | 125 | 127 | 129 | 55 | 131 |
| | (132) | (133) | (134) | (135) | (136) | (137) | (109) | (138) |
| mAb10929 | 137 | 139 | 141 | 143 | 145 | 147 | 149 | 151 |
| | (139) | (140) | (141) | (142) | (143) | (144) | (145) | (146) |
| mAb10930 | 167 | 169 | 171 | 173 | 175 | 129 | 55 | 177 |
| | (147) | (148) | (149) | (150) | (151) | (137) | (109) | (152) |
| mAb10931 | 167 | 169 | 171 | 173 | 175 | 129 | 55 | 177 |
| | (147) | (148) | (149) | (150) | (151) | (137) | (109) | (152) |
| mAb10932 | 185 | 187 | 26 | 189 | 191 | 75 | 194 | 196 |
| | (153) | (154) | (98) | (155) | (156) | (116) | (157) | (158) |
| mAb10933 | 202 | 204 | 206 | 208 | 210 | 212 | 55 | 214 |
| | (1) | (2) | (3) | (4) | (5) | (6) | (109) | (7) |
| mAb10934 | 220 | 222 | 224 | 226 | 228 | 230 | 194 | 232 |
| | (20) | (21) | (22) | (23) | (24) | (25) | (157) | (26) |
| mAb10935 | 238 | 24 | 26 | 240 | 242 | 244 | 194 | 246 |
| | (173) | (97) | (98) | (174) | (175) | (176) | (157) | (177) |
| mAb10936 | 252 | 254 | 256 | 258 | 260 | 129 | 55 | 262 |
| | (178) | (179) | (180) | (181) | (182) | (137) | (109) | (183) |
| mAb10937 | 268 | 270 | 272 | 274 | 276 | 129 | 55 | 278 |
| | (184) | (185) | (186) | (187) | (188) | (137) | (109) | (189) |
| mAb10940 | 284 | 169 | 286 | 288 | 290 | 292 | 294 | 296 |
| | (190) | (148) | (191) | (192) | (193) | (194) | (195) | (196) |
| mAb10938 | 302 | 24 | 26 | 304 | 306 | 308 | 194 | 310 |
| | (197) | (97) | (98) | (198) | (199) | (200) | (157) | (201) |
| mAb10939 | 316 | 187 | 319 | 321 | 323 | 325 | 55 | 327 |
| | (202) | (154) | (203) | (204) | (205) | (206) | (109) | (207) |
| mAb10941 | 333 | 85 | 26 | 336 | 338 | 340 | 294 | 296 |
| | (208) | (119) | (98) | (209) | (210) | (211) | (195) | (196) |
| mAb10942 | 185 | 187 | 26 | 189 | 191 | 75 | 194 | 196 |
| | (153) | (154) | (98) | (155) | (156) | (116) | (157) | (158) |
| mAb10943 | 202 | 204 | 206 | 208 | 210 | 212 | 55 | 214 |
| | (1) | (2) | (3) | (4) | (5) | (6) | (109) | (7) |
| mAb10944 | 220 | 222 | 224 | 226 | 228 | 230 | 194 | 232 |
| | (20) | (21) | (22) | (23) | (24) | (25) | (157) | (26) |
| mAb10945 | 238 | 24 | 26 | 240 | 242 | 244 | 194 | 246 |
| | (173) | (97) | (98) | (174) | (175) | (176) | (157) | (177) |
| mAb10946 | 252 | 254 | 256 | 258 | 260 | 129 | 55 | 262 |
| | (178) | (179) | (180) | (181) | (182) | (137) | (109) | (183) |
| mAb10947 | 268 | 270 | 272 | 274 | 276 | 129 | 55 | 278 |
| | (184) | (185) | (186) | (187) | (188) | (137) | (109) | (189) |
| mAb10948 | 302 | 24 | 26 | 304 | 306 | 308 | 194 | 310 |
| | (197) | (97) | (98) | (198) | (199) | (200) | (157) | (201) |
| mAb10949 | 316 | 187 | 319 | 321 | 323 | 325 | 55 | 327 |
| | (202) | (154) | (203) | (204) | (205) | (206) | (109) | (207) |
| mAb10951 | 333 | 85 | 26 | 336 | 338 | 340 | 294 | 296 |
| | (208) | (119) | (98) | (209) | (210) | (211) | (195) | (196) |
| mAb10950 | 284 | 169 | 286 | 288 | 290 | 292 | 294 | 296 |
| | (190) | (148) | (191) | (192) | (193) | (194) | (195) | (196) |
| mAb10954 | 366 | 85 | 26 | 370 | 372 | 244 | 194 | 375 |
| | (212) | (119) | (98) | (213) | (214) | (176) | (157) | (215) |
| mAb10955 | 381 | 383 | 26 | 385 | 387 | 389 | 194 | 310 |
| | (216) | (217) | (98) | (218) | (219) | (220) | (157) | (201) |
| mAb10956 | 396 | 187 | 26 | 399 | 401 | 389 | 194 | 403 |
| | (221) | (154) | (98) | (222) | (223) | (220) | (157) | (224) |
| mAb10957 | 409 | 411 | 26 | 414 | 416 | 53 | 55 | 418 |
| | (225) | (226) | (98) | (227) | (228) | (108) | (109) | (229) |
| mAb10958 | 366 | 85 | 26 | 370 | 372 | 244 | 194 | 375 |
| | (212) | (119) | (98) | (213) | (214) | (176) | (157) | (215) |
| mAb10959 | 381 | 383 | 26 | 385 | 387 | 389 | 194 | 310 |
| | (216) | (217) | (98) | (218) | (219) | (220) | (157) | (201) |
| mAb10960 | 396 | 187 | 26 | 399 | 401 | 389 | 194 | 403 |
| | (221) | (154) | (98) | (222) | (223) | (220) | (157) | (224) |
| mAb10961 | 409 | 411 | 26 | 414 | 416 | 53 | 55 | 418 |
| | (225) | (226) | (98) | (227) | (228) | (108) | (109) | (229) |
| mAb10964 | 432 | 434 | 436 | 438 | 440 | 442 | 55 | 445 |
| | (230) | (231) | (232) | (233) | (234) | (235) | (109) | (236) |
| mAb10965 | 451 | 453 | 26 | 455 | 457 | 459 | 34 | 462 |
| | (237) | (238) | (98) | (239) | (240) | (241) | (102) | (242) |
| mAb10966 | 468 | 187 | 26 | 470 | 472 | 389 | 194 | 474 |
| | (243) | (154) | (98) | (244) | (245) | (220) | (157) | (246) |
| mAb10967 | 480 | 24 | 483 | 485 | 487 | 389 | 194 | 489 |
| | (247) | (97) | (248) | (249) | (250) | (220) | (157) | (251) |
| mAb10969 | 495 | 497 | 499 | 501 | 503 | 389 | 194 | 214 |
| | (252) | (253) | (254) | (255) | (256) | (220) | (157) | (165) |
| mAb10970 | 510 | 24 | 26 | 512 | 514 | 516 | 194 | 518 |
| | (257) | (97) | (98) | (258) | (259) | (260) | (157) | (261) |
| mAb10971 | 524 | 411 | 26 | 528 | 530 | 532 | 55 | 534 |
| | (262) | (226) | (98) | (263) | (264) | (265) | (109) | (266) |
| mAb10973 | 432 | 434 | 436 | 438 | 440 | 442 | 55 | 445 |
| | (230) | (231) | (232) | (233) | (234) | (235) | (109) | (236) |
| mAb10974 | 451 | 453 | 26 | 455 | 457 | 459 | 34 | 462 |
| | (237) | (238) | (98) | (239) | (240) | (241) | (102) | (242) |
| mAb10975 | 468 | 187 | 26 | 470 | 472 | 389 | 194 | 474 |
| | (243) | (154) | (98) | (244) | (245) | (220) | (157) | (246) |
| mAb10976 | 480 | 24 | 483 | 485 | 487 | 389 | 194 | 489 |
| | (247) | (97) | (248) | (249) | (250) | (220) | (157) | (251) |
| mAb10977 | 548 | 550 | 552 | 554 | 556 | 558 | 294 | 560 |
| | (267) | (268) | (269) | (270) | (271) | (272) | (195) | (273) |
| mAb10978 | 495 | 497 | 499 | 501 | 503 | 389 | 194 | 214 |
| | (252) | (253) | (254) | (255) | (256) | (220) | (157) | (165) |
| mAb10979 | 510 | 24 | 26 | 512 | 514 | 516 | 194 | 518 |
| | (257) | (97) | (98) | (258) | (259) | (260) | (157) | (261) |
| mAb10980 | 524 | 411 | 26 | 528 | 530 | 532 | 55 | 534 |
| | (262) | (226) | (98) | (263) | (264) | (265) | (109) | (266) |
| mAb10981 | 548 | 550 | 552 | 554 | 556 | 558 | 294 | 560 |
| | (267) | (268) | (269) | (270) | (271) | (272) | (195) | (273) |
| mAb10982 | 574 | 187 | 576 | 578 | 580 | 582 | 584 | 586 |
| | (274) | (154) | (275) | (276) | (277) | (278) | (279) | (280) |
| mAb10983 | 574 | 187 | 576 | 578 | 580 | 582 | 584 | 586 |
| | (274) | (154) | (275) | (276) | (277) | (278) | (279) | (280) |
| mAb10984 | 594 | 596 | 26 | 598 | 600 | 12 | 14 | 602 |
| | (281) | (282) | (98) | (283) | (284) | (93) | (94) | (285) |
| mAb10985 | 608 | 169 | 610 | 612 | 614 | 616 | 584 | 618 |
| | (286) | (148) | (287) | (288) | (289) | (290) | (279) | (291) |
| mAb10986 | 624 | 626 | 26 | 628 | 630 | 582 | 632 | 634 |
| | (292) | (293) | (98) | (294) | (295) | (278) | (296) | (297) |
| mAb10987 | 640 | 642 | 499 | 644 | 646 | 648 | 650 | 652 |
| | (38) | (39) | (40) | (41) | (42) | (43) | (303) | (44) |
| mAb10988 | 658 | 660 | 662 | 664 | 666 | 668 | 670 | 672 |
| | (305) | (306) | (307) | (308) | (309) | (310) | (311) | (312) |
| mAb10989 | 678 | 680 | 682 | 684 | 686 | 688 | 650 | 690 |
| | (56) | (57) | (58) | (59) | (60) | (61) | (303) | (62) |
| mAb10990 | 594 | 596 | 26 | 598 | 600 | 12 | 14 | 602 |
| | (281) | (282) | (98) | (283) | (284) | (93) | (94) | (285) |
| mAb10991 | 608 | 169 | 610 | 612 | 614 | 616 | 584 | 618 |
| | (286) | (148) | (287) | (288) | (289) | (290) | (279) | (291) |
| mAb10992 | 624 | 626 | 26 | 628 | 630 | 582 | 632 | 634 |
| | (292) | (293) | (98) | (294) | (295) | (278) | (296) | (297) |
| mAb10993 | 640 | 642 | 499 | 644 | 646 | 648 | 650 | 652 |
| | (38) | (39) | (40) | (41) | (42) | (43) | (303) | (44) |
| mAb10994 | 658 | 660 | 662 | 664 | 666 | 668 | 670 | 672 |
| | (305) | (306) | (307) | (308) | (309) | (310) | (311) | (312) |
| mAb10995 | 678 | 680 | 682 | 684 | 686 | 688 | 650 | 690 |
| | (313) | (314) | (315) | (316) | (317) | (318) | (303) | (319) |
| mAb10996 | 708 | 24 | 26 | 711 | 713 | 129 | 55 | 715 |
| | (320) | (97) | (98) | (321) | (322) | (137) | (109) | (323) |
| mAb10997 | 708 | 24 | 26 | 711 | 713 | 129 | 55 | 715 |
| | (320) | (97) | (98) | (321) | (322) | (137) | (109) | (323) |
| mAb10998 | 723 | 187 | 26 | 725 | 727 | 129 | 55 | 729 |
| | (324) | (154) | (98) | (325) | (326) | (137) | (109) | (327) |
| mAb10999 | 723 | 187 | 26 | 725 | 727 | 129 | 55 | 729 |
| | (324) | (154) | (98) | (325) | (326) | (137) | (109) | (327) |
| mAb11000 | 737 | 24 | 26 | 739 | 741 | 743 | 55 | 745 |
| | (328) | (97) | (98) | (329) | (340) | (341) | (109) | (342) |
| mAb11001 | 737 | 24 | 26 | 739 | 741 | 743 | 55 | 745 |
| | (328) | (97) | (98) | (329) | (340) | (341) | (109) | (342) |
| mAb11002 | 753 | 24 | 26 | 755 | 713 | 129 | 55 | 715 |
| | (343) | (97) | (98) | (344) | (322) | (137) | (109) | (323) |
| mAb11003 | 753 | 24 | 26 | 755 | 713 | 129 | 55 | 715 |
| | (343) | (97) | (98) | (344) | (322) | (137) | (109) | (323) |
| mAb10914 | 44 | 46 | 26 | 49 | 51 | 53 | 55 | 57 |
| | (104) | (105) | (98) | (106) | (107) | (108) | (109) | (110) |
| mAb11004 | 764 | 766 | 499 | 768 | 770 | 91 | 55 | 772 |
| | (345) | (346) | (254) | (347) | (348) | (122) | (109) | (349) |
| mAb11005 | 764 | 766 | 499 | 768 | 770 | 91 | 55 | 772 |
| | (345) | (346) | (254) | (347) | (348) | (122) | (109) | (349) |
| mAb11006 | 780 | 782 | 26 | 784 | 786 | 53 | 55 | 788 |
| | (350) | (351) | (98) | (352) | (353) | (108) | (109) | (354) |
| mAb11007 | 780 | 782 | 26 | 784 | 786 | 53 | 55 | 788 |
| | (350) | (351) | (98) | (352) | (353) | (108) | (109) | (354) |
| mAb11008 | 796 | 24 | 26 | 798 | 800 | 53 | 55 | 802 |
| | (355) | (97) | (98) | (356) | (357) | (108) | (109) | (358) |
| mAb11009 | 796 | 24 | 26 | 798 | 800 | 53 | 55 | 802 |
| | (355) | (97) | (98) | (356) | (357) | (108) | (109) | (358) |
| mAb11010 | 810 | 812 | 814 | 816 | 818 | 129 | 820 | 822 |
| | (359) | (360) | (361) | (362) | (363) | (137) | (364) | (365) |
| mAb11011 | 810 | 812 | 814 | 816 | 818 | 129 | 820 | 822 |
| | (359) | (360) | (361) | (362) | (363) | (137) | (364) | (365) |

In some embodiments, the spike protein ABDs comprise an amino acid sequence set forth in Table 4 below. In some aspects, the spike protein ABD comprises both heavy and light chain CDRs of an antibody set forth in Table 4 below. In certain aspects, a spike protein ABD comprises a VH having the amino acid sequence of the VH of an antibody set forth in Table 4 and a VL having the amino acid sequence of the VL of the same antibody as set forth in Table 4. In other aspects, a spike protein ABD comprises a VH having the amino acid sequence of the VH of an antibody set forth in Table 4 and a universal light chain VL sequence. The initial sequence identifiers for Table 4 are in relation to the sequence listing in WO 2023/287875A1, which sequence identifiers are incorporated by reference herein, while sequence identifiers presented parenthetically are those of the disclosure.

TABLE 4
SEQ ID NO in WO2023/287875A1

| Antibody | | CDR- | CDR- | CDR- | | CDR- | CDR- | CDR- |
|---|---|---|---|---|---|---|---|---|
| Designation | VH | H1 | H2 | H3 | VL | L1 | L2 | L3 |
| mAb15163 | 2 | 4 | 6 | 8 | 10 | 12 | 14 | 16 |
| | (366) | (367) | (368) | (369) | (370) | (371) | (372) | (373) |
| mAb15164 | 22 | 24 | 26 | 28 | 30 | 32 | 34 | 36 |
| | (374) | (375) | (376) | (377) | (378) | (379) | (380) | (381) |
| mAb15165 | 42 | 44 | 26 | 47 | 49 | 51 | 34 | 36 |
| | (382) | (383) | (376) | (384) | (385) | (386) | (380) | (381) |
| mAb15166 | 58 | 60 | 62 | 64 | 66 | 51 | 68 | 36 |
| | (387) | (388) | (389) | (390) | (391) | (386) | (392) | (381) |
| mAb15167 | 74 | 76 | 78 | 80 | 82 | 84 | 86 | 88 |
| | (393) | (394) | (395) | (396) | (397) | (398) | (399) | (400) |
| mAb15170 | 94 | 96 | 98 | 100 | 102 | 104 | 106 | 108 |
| | (401) | (402) | (403) | (404) | (405) | (406) | (407) | (408) |
| mAb14296 | 114 | 116 | 118 | 120 | 122 | 124 | 126 | 128 |
| | (409) | (410) | (411) | (412) | (413) | (414) | (415) | (417) |
| mAb14297 | 134 | 136 | 138 | 140 | 142 | 144 | 126 | 146 |
| | (418) | (419) | (420) | (421) | (422) | (423) | (415) | (424) |
| mAb14312 | 152 | 154 | 156 | 158 | 160 | 162 | 164 | 166 |
| | (425) | (426) | (427) | (428) | (429) | (430) | (431) | (432) |
| mAb14313 | 172 | 174 | 176 | 178 | 180 | 182 | 184 | 186 |
| | (433) | (434) | (435) | (436) | (437) | (438) | (439) | (440) |
| mAb14314 | 192 | 194 | 196 | 198 | 200 | 202 | 204 | 206 |
| | (441) | (442) | (443) | (444) | (445) | (446) | (447) | (448) |
| mAb14315 | 212 | 214 | 216 | 218 | 220 | 222 | 126 | 224 |
| | (449) | (450) | (451) | (452) | (453) | (454) | (415) | (455) |
| mAb14316 | 230 | 232 | 234 | 236 | 238 | 240 | 242 | 244 |
| | (456) | (457) | (458) | (459) | (460) | (461) | (462) | (463) |
| mAb15150 | 250 | 252 | 254 | 256 | 258 | 260 | 262 | 264 |
| | (464) | (465) | (466) | (467) | (468) | (469) | (470) | (471) |
| mAb15151 | 270 | 272 | 274 | 276 | 278 | 280 | 106 | 282 |
| | (472) | (473) | (474) | (475) | (476) | (477) | (407) | (478) |
| mAb15156 | 288 | 290 | 292 | 294 | 296 | 298 | 300 | 302 |
| | (479) | (480) | (481) | (482) | (483) | (484) | (485) | (486) |
| mAb15157 | 308 | 310 | 312 | 314 | 316 | 318 | 320 | 322 |
| | (487) | (488) | (489) | (490) | (491) | (492) | (493) | (494) |
| mAb15158 | 328 | 330 | 332 | 334 | 336 | 338 | 106 | 340 |
| | (495) | (496) | (497) | (498) | (499) | (500) | (407) | (501) |
| mAb15159 | 346 | 96 | 98 | 350 | 352 | 354 | 106 | 356 |
| | (752) | (402) | (403) | (502) | (503) | (504) | (407) | (505) |
| mAb15160 | 362 | 364 | 366 | 368 | 370 | 372 | 106 | 374 |
| | (506) | (507) | (508) | (509) | (510) | (511) | (407) | (512) |
| mAb15161 | 380 | 382 | 384 | 386 | 388 | 390 | 392 | 394 |
| | (513) | (514) | (515) | (516) | (517) | (518) | (519) | (520) |
| mAb15162 | 400 | 402 | 98 | 405 | 407 | 409 | 242 | 411 |
| | (521) | (522) | (403) | (523) | (524) | (525) | (462) | (526) |
| mAb14280 | 417 | 419 | 421 | 423 | 425 | 427 | 14 | 429 |
| | (527) | (528) | (529) | (530) | (531) | (532) | (372) | (533) |
| mAb14281 | 435 | 437 | 138 | 440 | 442 | 444 | 446 | 448 |
| | (534) | (535) | (420) | (536) | (537) | (538) | (539) | (540) |
| mAb14282 | 454 | 456 | 458 | 460 | 462 | 84 | 465 | 467 |
| | (541) | (542) | (543) | (544) | (545) | (398) | (546) | (547) |
| mAb14283 | 473 | 475 | 477 | 479 | 481 | 483 | 485 | 487 |
| | (548) | (549) | (550) | (551) | (552) | (553) | (554) | (555) |
| mAb14284 | 493 | 495 | 497 | 499 | 501 | 503 | 505 | 507 |
| | (556) | (557) | (558) | (559) | (560) | (561) | (562) | (563) |
| mAb14285 | 513 | 515 | 517 | 519 | 521 | 523 | 525 | 36 |
| | (564) | (565) | (566) | (567) | (568) | (569) | (570) | (381) |
| mAb14286 | 531 | 533 | 535 | 537 | 539 | 483 | 542 | 544 |
| | (571) | (572) | (573) | (574) | (575) | (553) | (576) | (577) |
| mAb14287 | 550 | 552 | 554 | 556 | 558 | 84 | 14 | 560 |
| | (578) | (579) | (580) | (581) | (582) | (398) | (372) | (583) |
| mAb14288 | 566 | 568 | 570 | 572 | 574 | 576 | 578 | 580 |
| | (584) | (585) | (586) | (587) | (588) | (589) | (590) | (591) |
| mAb14289 | 586 | 588 | 590 | 592 | 594 | 596 | 126 | 598 |
| | (592) | (593) | (594) | (595) | (596) | (597) | (415) | (598) |
| mAb14290 | 604 | 606 | 608 | 610 | 612 | 614 | 126 | 616 |
| | (599) | (600) | (601) | (602) | (603) | (604) | (415) | (605) |
| mAb14291 | 622 | 624 | 626 | 628 | 630 | 632 | 634 | 636 |
| | (606) | (607) | (608) | (609) | (610) | (611) | (612) | (613) |
| mAb14292 | 642 | 644 | 646 | 648 | 650 | 652 | 634 | 655 |
| | (614) | (615) | (616) | (617) | (618) | (619) | (612) | (620) |
| mAb14293 | 661 | 663 | 665 | 667 | 669 | 124 | 126 | 671 |
| | (621) | (622) | (623) | (624) | (625) | (414) | (415) | (626) |
| mAb14295 | 677 | 679 | 78 | 682 | 684 | 686 | 126 | 688 |
| | (627) | (628) | (395) | (629) | (630) | (631) | (415) | (632) |
| mAb13459 | 694 | 696 | 698 | 700 | 702 | 704 | 706 | 708 |
| | (633) | (634) | (635) | (636) | (637) | (638) | (639) | (640) |
| mAb14230 | 714 | 696 | 716 | 718 | 720 | 722 | 126 | 724 |
| | (641) | (634) | (642) | (643) | (644) | (645) | (415) | (646) |
| mAb14231 | 730 | 732 | 734 | 736 | 738 | 740 | 106 | 742 |
| | (647) | (648) | (649) | (650) | (651) | (652) | (407) | (653) |
| mAb14232 | 748 | 750 | 497 | 752 | 754 | 756 | 505 | 758 |
| | (654) | (655) | (558) | (656) | (657) | (658) | (562) | (659) |
| mAb14233 | 764 | 766 | 768 | 770 | 772 | 774 | 776 | 778 |
| | (660) | (661) | (662) | (663) | (664) | (665) | (666) | (667) |
| mAb14234 | 784 | 786 | 788 | 790 | 792 | 794 | 796 | 798 |
| | (668) | (669) | (670) | (671) | (672) | (673) | (674) | (675) |
| mAb14235 | 804 | 806 | 497 | 808 | 810 | 812 | 505 | 814 |
| | (676) | (677) | (558) | (678) | (679) | (680) | (562) | (681) |
| mAb14247 | 820 | 822 | 497 | 825 | 827 | 756 | 14 | 829 |
| | (682) | (683) | (558) | (684) | (685) | (658) | (372) | (686) |
| mAb14248 | 835 | 837 | 839 | 841 | 843 | 845 | 847 | 849 |
| | (687) | (688) | (689) | (690) | (691) | (692) | (693) | (694) |
| mAb14249 | 855 | 857 | 859 | 861 | 863 | 865 | 106 | 867 |
| | (695) | (696) | (697) | (698) | (699) | (700) | (407) | (701) |
| mAb14255 | 873 | 76 | 876 | 878 | 880 | 84 | 86 | 36 |
| | (702) | (394) | (703) | (704) | (705) | (398) | (399) | (381) |
| mAb14256 | 887 | 889 | 891 | 893 | 895 | 897 | 164 | 899 |
| | (706) | (707) | (708) | (709) | (710) | (711) | (431) | (712) |
| mAb14257 | 905 | 154 | 908 | 910 | 912 | 914 | 916 | 166 |
| | (713) | (426) | (714) | (715) | (716) | (717) | (718) | (432) |
| mAb14258 | 922 | 924 | 926 | 928 | 930 | 576 | 933 | 935 |
| | (719) | (720) | (721) | (722) | (723) | (589) | (724) | (725) |
| mAb14259 | 941 | 943 | 945 | 947 | 949 | 951 | 634 | 655 |
| | (726) | (727) | (728) | (729) | (730) | (731) | (612) | (620) |
| mAb14260 | 957 | 959 | 961 | 963 | 965 | 967 | 969 | 971 |
| | (732) | (733) | (734) | (735) | (736) | (737) | (738) | (739) |
| mAb13457 | 694 | 696 | 698 | 700 | 977 | 704 | 706 | 979 |
| | (633) | (634) | (635) | (636) | (740) | (638) | (639) | (741) |
| mAb13458 | 694 | 696 | 698 | 700 | 983 | 704 | 706 | 985 |
| | (633) | (634) | (635) | (636) | (742) | (638) | (639) | (743) |
| mAb14294 | 989 | 991 | 993 | 995 | 997 | 999 | 1001 | 1003 |
| | (744) | (745) | (746) | (747) | (748) | (749) | (750) | (751) |
| mAb17090 | 493 | 495 | 497 | 499 | 501 | 503 | 505 | 507 |
| | (556) | (557) | (558) | (559) | (560) | (561) | (562) | (563) |
| mAb15160_2 | 362 | 364 | 366 | 368 | 370 | 372 | 106 | 374 |
| | (506) | (507) | (508) | (509) | (510) | (511) | (407) | (512) |

In some embodiments, a spike protein ABD of a multivalent anti-spike protein binding molecule comprises the heavy and light chain CDRs of antibody “mAb14287” as set forth in Table 4. Accordingly, in some embodiments, the spike protein ABD comprises a VH comprising CDR-H1, CDR-H2, and CDR-H3 having the amino acid sequences of SEQ ID NOs: 579, 580, and 581, respectively, and a VL comprising CDR-L1, CDR-L2, and CDR-L3 having the amino acid sequences of SEQ ID NOs: 398, 372, and 583, respectively. In some embodiments, the spike protein ABD comprises a VH having at least 90%, at least 95%, at least 96%, at least 97%, at least 98%, at least 99%, or 100% sequence identity to the amino acid sequence of SEQ ID NO:578 and a VL having at least 90%, at least 95%, at least 96%, at least 97%, at least 98%, at least 99%, or 100% sequence identity to the amino acid sequence of SEQ ID NO:582. In some embodiments, the spike protein ABD comprises a VH comprising the amino acid sequence of SEQ ID NO:578 and a VL comprising the amino acid sequence of SEQ ID NO:582.

In some embodiments, a spike protein ABD of a multivalent anti-spike protein binding molecule comprises the heavy and light chain CDRs of antibody “mAb 15160” as set forth in Table 4. Accordingly, in some embodiments, the spike protein ABD comprises a VH comprising CDR-H1, CDR-H2, and CDR-H3 having the amino acid sequences of SEQ ID NOs: 507, 508, and 509, respectively, and a VL comprising CDR-L1, CDR-L2, and CDR-L3 having the amino acid sequences of SEQ ID NOs: 511, 407, and 512, respectively. In some embodiments, the spike protein ABD comprises a VH having at least 90%, at least 95%, at least 96%, at least 97%, at least 98%, at least 99%, or 100% sequence identity to the amino acid sequence of SEQ ID NO:506 and a VL having at least 90%, at least 95%, at least 96%, at least 97%, at least 98%, at least 99%, or 100% sequence identity to the amino acid sequence of SEQ ID NO:510. In some embodiments, the spike protein ABD comprises a VH comprising the amino acid sequence of SEQ ID NO:506 and a VL comprising the amino acid sequence of SEQ ID NO:510.

In some embodiments, a spike protein ABD of a multivalent anti-spike protein binding molecule comprises the heavy and light chain CDRs of antibody “mAb14315” as set forth in Table 4. Accordingly, in some embodiments, the spike protein ABD comprises a VH comprising CDR-H1, CDR-H2, and CDR-H3 having the amino acid sequences of SEQ ID NOs: 450, 451, and 452, respectively, and a VL comprising CDR-L1, CDR-L2, and CDR-L3 having the amino acid sequences of SEQ ID NOs: 454, 415, and 455, respectively. In some embodiments, the spike protein ABD comprises a VH having at least 90%, at least 95%, at least 96%, at least 97%, at least 98%, at least 99%, or 100% sequence identity to the amino acid sequence of SEQ ID NO:449 and a VL having at least 90%, at least 95%, at least 96%, at least 97%, at least 98%, at least 99%, or 100% sequence identity to the amino acid sequence of SEQ ID NO:453. In some embodiments, the spike protein ABD comprises a VH comprising the amino acid sequence of SEQ ID NO:449 and a VL comprising the amino acid sequence of SEQ ID NO:453.

Exemplary formats of spike protein ABDs are disclosed in Section 6.4, and include Fabs (e.g., as described in Section 6.4.1) and scFvs (e.g., as described in Section 6.4.2).

In some embodiments, the spike protein ABD is in the form of a Fab or an scFv.

In further embodiments, all spike protein ABDs in a multivalent spike protein binding molecule of the disclosure (e.g., a tetravalent spike protein binding molecule of the disclosure) are Fabs.

In some embodiments, the multivalent spike protein binding molecule is monospecific, wherein all ABDs bind to the same epitope. The ABDs in a monospecific multivalent spike protein binding molecule may all comprise the same CDR sequences or the same VH and VL sequences. In some embodiments, the ABDs of a multispecific multivalent spike protein binding molecule are configured as shown in FIG. 1A or FIG. 1C.

In some embodiments, the multivalent spike protein binding molecule is multispecific, wherein at least two ABDs bind to different epitopes. At least two ABDs in a multispecific multivalent spike protein binding molecule comprise different CDR sequences. In some embodiments, the multispecific multivalent spike protein binding molecule is tetravalent, with a first pair of ABDs sharing the same CDR sequences or the same VH and VL sequences and a second pair of ABDs sharing the CDR sequences or the same VH and VL sequences (which differ from the CDR sequences of the first pair of ABDs). In some embodiments, the ABDs of a multispecific multivalent spike protein binding molecule are configured as shown in FIG. 1B or FIG. 1D.

TABLE S
Sequences of Tables 3 and 4 Excluded
from ST.26-Formatted Sequence Listing

| SEQ ID NO | Sequence |
|---|---|
| 94 | DNN |
| 102 | KAS |
| 109 | AAS |
| 130 | WAS |
| 145 | GVS |
| 157 | DAS |
| 195 | GAS |
| 279 | GNS |
| 296 | GNT |
| 303 | DVS |
| 311 | SNN |
| 364 | TAS |
| 372 | EVS |
| 380 | EGN |
| 392 | EGT |
| 399 | EDS |
| 407 | AAS |
| 415 | GAS |
| 431 | GNS |
| 439 | SND |
| 447 | DND |
| 462 | DAS |
| 470 | GAT |
| 485 | SDN |
| 493 | VNN |
| 519 | KAS |
| 539 | DKN |
| 546 | ELT |
| 554 | DVT |
| 562 | EVT |
| 570 | EGS |
| 576 | DVS |
| 590 | ENN |
| 612 | LGS |
| 639 | SAS |
| 666 | KDS |
| 674 | GNT |
| 693 | KIS |
| 718 | GHT |
| 724 | RNN |
| 738 | WAS |
| 750 | GAS |

### 6.4. Spike Protein Antigen Binding Domain Formats

In certain aspects, the multivalent anti-spike protein binding molecules of the disclosure comprise an ABD of an anti-spike protein antibody that retains specific binding to an antigenic determinant. In one embodiment, the spike protein ABD is a naturally occurring (e.g., by protease cleavage) or engineered fragment of an immunoglobulin. Antibody fragments include, but are not limited to, VH (or VH fragments), VL (or VL fragments), Fab fragments, F(ab')2 fragments, scFv fragments, Fv fragments, minibodies, diabodies, triabodies, and tetrabodies.

### 6.4.1. Fab

Fab domains were traditionally produced by proteolytic cleavage of immunoglobulin molecules using enzymes such as papain. The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding site. A disulfide bond between the two constant domains can further stabilize the Fab domain.

For the anti-spike protein binding antibodies of the disclosure that are not homodimeric, particularly when the light chains of the anti-spike protein antibody are not common or universal light chains, it is advantageous to use Fab heterodimerization strategies to permit the correct association of Fab domains belonging to the same antigen-binding domain and minimize aberrant pairing of Fab domains belonging to different antigen-binding domains. For example, the Fab heterodimerization strategies shown in Table 5 below can be used:

TABLE 5
Fab Heterodimerization Strategies

| STRATEGY | VH | CH1 | VL | CL | REFERENCE |
|---|---|---|---|---|---|
| CrossMabCH1- | WT | CL domain | WT | CH1 domain | Schaefer et al., 2011, |
| CL | | | | | Cancer Cell 2011; |
| | | | | | 20: 472-86; |
| | | | | | PMID:22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MABD |
| | | | | | 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, |
| | | | | | MABD 7: 377-89; Mazor |
| | | | | | et al., 2015, MABD |
| | | | | | 7: 461-669. |
| Domain | WT | CH3 + knob or | WT | CH3 + hole or | Wozniak-Knopp et al., |
| exchanged | | hole mutation | | knob mutation | 2018, PloS |
| | | | | | ONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014, Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MABD 7:377-89).

Fab domains can also be modified by replacing the CH1 domain and CL domain with alternative domains that promote correct assembly. For example, Wu et al., 2015, MABD 7:364-76, describes substituting the CH1 domain with the constant domain of the T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

6.4.2. scFv

Single chain Fv or “scFv” antibody fragments comprise the VH and VL domains of an antibody in a single polypeptide chain, are capable of being expressed as a single chain polypeptide and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFv are the linkers identified in Section 6.6.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.6 (typically a repeat of a sequence containing the amino acids glycine and serine, such as the amino acid sequence (Gly4˜Ser)3 (SEQ ID NO: 74), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.5. Multimerization Moiety

In some embodiments, the ABDs of the multivalent anti-spike protein binding molecules of the disclosure include and/or are operably linked by one or more multimerization moieties, for example one or more multimerization moieties that comprise or consist of an Fc domain.

In certain embodiments, a multivalent anti-spike protein binding molecule of the disclosure comprises a single multimerization moiety (e.g., a single Fc domain) but more typically comprises two or more multimerization moieties (e.g., two or more Fc domains that can associate to form an Fc region). In some embodiments, the multivalent anti-spike protein binding molecule is a dimer, and the Fc region comprises two IgG-derived Fc domains, for example as described in Section 6.5.1.

### 6.5.1. Fc Domains

The multivalent anti-spike protein binding molecules of the disclosure can include an Fc domain, or a pair of Fc domains that associate to form an Fc region, derived from any suitable species operably linked to a spike protein ABD. In one embodiment the Fc domain is derived from a human Fc domain. In preferred embodiments, the spike protein ABD is fused to an IgG Fc domain.

The Fc domains that can be incorporated into multivalent anti-spike protein binding molecules can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In another embodiment, the Fc domain is derived from IgG4.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In the multivalent anti-spike protein binding molecules of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

The heavy chain constant domains for use in producing an Fc region for the multivalent anti-spike protein binding molecules of the present disclosure may include variants of the naturally occurring constant domains described above. Such variants may comprise one or more amino acid variations compared to wildtype constant domains. In one example, the Fc region of the present disclosure comprises at least one constant domain that varies in sequence from the wildtype constant domain. It will be appreciated that the variant constant domains may be longer or shorter than the wildtype constant domain.

The Fc domains that are incorporated into the multivalent anti-spike protein binding molecules of the present disclosure may comprise one or more modifications that alter the functional properties of the proteins, for example, binding to Fc-receptors such as FcRn or leukocyte receptors, binding to complement, modified disulfide bond architecture, or altered glycosylation patterns. Exemplary Fc modifications that alter effector function are described in Section 6.5.1.1.

The Fc domains can also be altered to include modifications that improve

manufacturability of asymmetric multivalent anti-spike protein binding molecules, for example by allowing heterodimerization, which is the preferential pairing of non-identical Fc domains over identical Fc domains. Heterodimerization permits the production of multivalent anti-spike protein binding molecules in which different polypeptide components are connected to one another by an Fc region containing Fc domains that differ in sequence. Examples of heterodimerization strategies are exemplified in Section 6.5.1.2.

It will be appreciated that any of the modifications mentioned above can be combined in any suitable manner to achieve the desired functional properties and/or combined with other modifications to alter the properties of the multivalent anti-spike protein binding molecules.

6.5.1.1. Fc Domains with Altered Effector Function

In some embodiments, the Fc domain comprises one or more amino acid substitutions that reduce binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In one embodiment, the Fc domain (e.g., an Fc domain of a multivalent anti-spike protein binding molecule polypeptide chain or the Fc region (e.g., one or both Fc domains of a multivalent anti-spike protein binding construct that can associate to form an Fc region) comprises an amino acid substitution at a position selected from the group of E233, L234, L235, N297, P331 and P329 (numberings according to Kabat EU index). In a more specific embodiment, the Fc domain or the Fc region comprises an amino acid substitution at a position selected from the group of L234, L235 and P329 (numberings according to Kabat EU index). In some embodiments, the Fc domain or the Fc region comprises the amino acid substitutions L234A and L235A (numberings according to Kabat EU index). In one such embodiment, the Fc domain or region is an IgD Fc domain or region, particularly a human IgD Fc domain or region. In one embodiment, the Fc domain or the Fc region comprises an amino acid substitution at position P329. In a more specific embodiment, the amino acid substitution is P329A or P329G, particularly P329G (numberings according to Kabat EU index). In one embodiment, the Fc domain or the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain or the Fc region comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table 6 below: In some embodiments, the Fc domain includes only the bolded portion of the sequences shown below:

TABLE 6

| Fc Domain | Sequence |
|---|---|
| SEQ ID NO: 1 of | Asp Lys Arg Val Glu Ser Lys Tyr Gly Pro Cys Pro Pro Cys Pro |
| WO2014/121087 | Ala Pro Pro Val Ala Gly Pro Ser Val Phe Leu Phe Pro Pro Lys |
| | Pro Lys Asp Thr Leu Met Ile Ser Arg Thr Pro Glu Val Thr Cys |
| | Val Val Val Asp Val Ser Gln Glu Asp Pro Glu Val Gln Phe Asn |
| | Trp Tyr Val Asp Gly Val Glu Val His Asn Ala Lys Thr Lys Pro |
| | Arg Glu Glu Gln Phe Asn Ser Thr Tyr Arg Val Val Ser Val Leu |
| | Thr Val Leu His Gln Asp Trp Leu Asn Gly Lys Glu Tyr Lys Cys |
| | Lys Val Ser Asn Lys Gly Leu Pro Ser Ser Ile Glu Lys Thr Ile |
| | Ser Lys Ala Lys Gly Gln Pro Arg Glu Pro Gln Val Tyr Thr Leu |
| | Pro Pro Ser Gln Glu Glu Met Thr Lys Asn Gln Val Ser Leu Thr |
| | Cys Leu Val Lys Gly Phe Tyr Pro Ser Asp Ile Ala Val Glu Trp |
| | Glu Ser Asn Gly Gln Pro Glu Asn Asn Tyr Lys Thr Thr Pro Pro |
| | Val Leu Asp Ser Asp Gly Ser Phe Phe Leu Tyr Ser Arg Leu Thr |
| | Val Asp Lys Ser Arg Trp Gln Glu Gly Asn Val Phe Ser Cys Ser |
| | Val Met His Glu Ala Leu His Asn His Tyr Thr Gln Lys Ser Leu |
| | Ser Leu Ser Leu Gly Lys (SEQ ID NO: 75) |
| SEQ ID NO: 2 of | Asp Lys Lys Val Glu Pro Lys Ser Cys Asp Lys Thr His Thr Cys |
| WO2014/121087 | Pro Pro Cys Pro Ala Pro Pro Val Ala Gly Pro Ser Val Phe Leu |
| | Phe Pro Pro Lys Pro Lys Asp Thr Leu Met Ile Ser Arg Thr Pro |
| | Glu Val Thr Cys Val Val Val Asp Val Ser Gln Glu Asp Pro Glu |
| | Val Gln Phe Asn Trp Tyr Val Asp Gly Val Glu Val His Asn Ala |
| | Lys Thr Lys Pro Arg Glu Glu Gln Phe Asn Ser Thr Tyr Arg Val |
| | Val Ser Val Leu Thr Val Leu His Gln Asp Trp Leu Asn Gly Lys |
| | Glu Tyr Lys Cys Lys Val Ser Asn Lys Gly Leu Pro Ser Ser Ile |
| | Glu Lys Thr Ile Ser Lys Ala Lys Gly Gln Pro Arg Glu Pro Gln |
| | Val Tyr Thr Leu Pro Pro Ser Arg Asp Glu Leu Thr Lys Asn Gln |
| | Val Ser Leu Thr Cys Leu Val Lys Gly Phe Tyr Pro Ser Asp Ile |
| | Ala Val Glu Trp Glu Ser Asn Gly Gln Pro Glu Asn Asn Tyr Lys |
| | Thr Thr Pro Pro Val Leu Asp Ser Asp Gly Ser Phe Phe Leu Tyr |
| | Ser Lys Leu Thr Val Asp Lys Ser Arg Trp Gln Gln Gly Asn Val |
| | Phe Ser Cys Ser Val Met His Glu Ala Leu His Asn His Tyr Thr |
| | Gln Lys Ser Leu Ser Leu Ser Pro Gly Lys (SEQ ID NO: 18) |
| SEQ ID NO: 30 of | Ala Ser Thr Lys Gly Pro Ser Val Phe Pro Leu Ala Pro Ser Ser |
| WO2014/121087 | Lys Ser Thr Ser Gly Gly Thr Ala Ala Leu Gly Cys Leu Val Lys |
| | Asp Tyr Phe Pro Glu Pro Val Thr Val Ser Trp Asn Ser Gly Ala |
| | Leu Thr Ser Gly Val His Thr Phe Pro Ala Val Leu Gln Ser Ser |
| | Gly Leu Tyr Ser Leu Ser Ser Val Val Thr Val Pro Ser Ser Ser |
| | Leu Gly Thr Gln Thr Tyr Ile Cys Asn Val Asn His Lys Pro Ser |
| | Asn Thr Lys Val Asp Lys Lys Val Glu Pro Lys Ser Cys Asp |
| | Lys Thr His Thr Cys Pro Pro Cys Pro Ala Pro Pro Val Ala |
| | Gly Pro Ser Val Phe Leu Phe Pro Pro Lys Pro Lys Asp Thr |
| | Leu Met Ile Ser Arg Thr Pro Glu Val Thr Cys Val Val Val |
| | Asp Val Ser Gln Glu Asp Pro Glu Val Gln Phe Asn Trp Tyr |
| | Val Asp Gly Val Glu Val His Asn Ala Lys Thr Lys Pro Arg |
| | Glu Glu Gln Phe Asn Ser Thr Tyr Arg Val Val Ser Val Leu |
| | Thr Val Leu His Gln Asp Trp Leu Asn Gly Lys Glu Tyr Lys |
| | Cys Lys Val Ser Asn Lys Gly Leu Pro Ser Ser Ile Glu Lys |
| | Thr Ile Ser Lys Ala Lys Gly Gln Pro Arg Glu Pro Gln Val Tyr |
| | Thr Leu Pro Pro Ser Arg Asp Glu Leu Thr Lys Asn Gln Val |
| | Ser Leu Thr Cys Leu Val Lys Gly Phe Tyr Pro Ser Asp Ile |
| | Ala Val Glu Trp Glu Ser Asn Gly Gln Pro Glu Asn Asn Tyr |
| | Lys Thr Thr Pro Pro Val Leu Asp Ser Asp Gly Ser Phe Phe |
| | Leu Tyr Ser Lys Leu Thr Val Asp Lys Ser Arg Trp Gln Gln |
| | Gly Asn Val Phe Ser Cys Ser Val Met His Glu Ala Leu His |
| | Asn His Tyr Thr Gln Lys Ser Leu Ser Leu Ser Pro Gly Lys |
| | (SEQ ID NO: 76) |
| SEQ ID NO: 31 of | Ala Ser Thr Lys Gly Pro Ser Val Phe Pro Leu Ala Pro Cys Ser |
| WO2014/121087 | Arg Ser Thr Ser Glu Ser Thr Ala Ala Leu Gly Cys Leu Val Lys |
| | Asp Tyr Phe Pro Glu Pro Val Thr Val Ser Trp Asn Ser Gly Ala |
| | Leu Thr Ser Gly Val His Thr Phe Pro Ala Val Leu Gln Ser Ser |
| | Gly Leu Tyr Ser Leu Ser Ser Val Val Thr Val Pro Ser Ser Ser |
| | Leu Gly Thr Lys Thr Tyr Thr Cys Asn Val Asp His Lys Pro Ser |
| | Asn Thr Lys Val Asp Lys Arg Val Glu Ser Lys Tyr Gly Pro Pro |
| | Cys Pro Pro Cys Pro Ala Pro Pro Val Ala Gly Pro Ser Val |
| | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala |
| | Lys Gly Gln Pro Arg Glu Pro Gln Val Tyr Thr Leu Pro Pro |
| | Ser Gln Glu Glu Met Thr Lys Asn Gln Val Ser Leu Thr Cys |
| | Leu Val Lys Gly Phe Tyr Pro Ser Asp Ile Ala Val Glu Trp |
| | Glu Ser Asn Gly Gln Pro Glu Asn Asn Tyr Lys Thr Thr Pro |
| | Pro Val Leu Asp Ser Asp Gly Ser Phe Phe Leu Tyr Ser Arg |
| | Leu Thr Val Asp Lys Ser Arg Trp Gln Glu Gly Asn Val Phe |
| | Ser Cys Ser Val Met His Glu Ala Leu His Asn His Tyr Thr |
| | Gln Lys Ser Leu Ser Leu Ser Leu Gly Lys (SEQ ID NO: 77) |
| SEQ ID NO: 37 of | Ala Ser Thr Lys Gly Pro Ser Val Phe Pro Leu Ala Pro Ser Ser |
| WO2014/121087 | Lys Ser Thr Ser Gly Gly Thr Ala Ala Leu Gly Cys Leu Val Lys |
| | Asp Tyr Phe Pro Glu Pro Val Thr Val Ser Trp Asn Ser Gly Ala |
| | Leu Thr Ser Gly Val His Thr Phe Pro Ala Val Leu Gln Ser Ser |
| | Gly Leu Tyr Ser Leu Ser Ser Val Val Thr Val Pro Ser Ser Ser |
| | Leu Gly Thr Gln Thr Tyr Ile Cys Asn Val Asn His Lys Pro Ser |
| | Asn Thr Lys Val Asp Lys Lys Val Glu Pro Lys Ser Cys Asp |
| | Lys Thr His Thr Cys Pro Pro Cys Pro Ala Pro Pro Val Ala |
| | Gly Pro Ser Val Phe Leu Phe Pro Pro Lys Pro Lys Asp Thr |
| | Leu Met Ile Ser Arg Thr Pro Glu Val Thr Cys Val Val Val |
| | Asp Val Ser Gln Glu Asp Pro Glu Val Gln Phe Asn Trp Tyr |
| | Val Asp Gly Val Glu Val His Asn Ala Lys Thr Lys Pro Arg |
| | Glu Glu Gln Phe Asn Ser Thr Tyr Arg Val Val Ser Val Leu |
| | Thr Val Leu His Gln Asp Trp Leu Asn Gly Lys Glu Tyr Lys |
| | Cys Lys Val Ser Asn Lys Gly Leu Pro Ser Ser Ile Glu Lys |
| | Thr Ile Ser Lys Ala Lys Gly Gln Pro Arg Glu Pro Gln Val Tyr |
| | Thr Leu Pro Pro Ser Arg Asp Glu Leu Thr Lys Asn Gln Val |
| | Ser Leu Thr Cys Leu Val Lys Gly Phe Tyr Pro Ser Asp Ile |
| | Ala Val Glu Trp Glu Ser Asn Gly Gln Pro Glu Asn Asn |
| | Tyr Lys Thr Thr Pro Pro Val Leu Asp Ser Asp Gly Ser Phe |
| | Phe Leu Tyr Ser Lys Leu Thr Val Asp Lys Ser Arg Trp Gln |
| | Gln Gly Asn Val Phe Ser Cys Ser Val Met His Glu Ala Leu |
| | His Asn Arg Phe Thr Gln Lys Ser Leu Ser Leu Ser Pro Gly |
| | Lys (SEQ ID NO: 78) |
| SEQ ID NO: 38 of | Ala Ser Thr Lys Gly Pro Ser Val Phe Pro Leu Ala Pro Cys Ser |
| WO2014/121087 | Arg Ser Thr Ser Glu Ser Thr Ala Ala Leu Gly Cys Leu Val Lys |
| | Asp Tyr Phe Pro Glu Pro Val Thr Val Ser Trp Asn Ser Gly Ala |
| | Leu Thr Ser Gly Val His Thr Phe Pro Ala Val Leu Gln Ser Ser |
| | Gly Leu Tyr Ser Leu Ser Ser Val Val Thr Val Pro Ser Ser Ser |
| | Leu Gly Thr Lys Thr Tyr Thr Cys Asn Val Asp His Lys Pro Ser |
| | Asn Thr Lys Val Asp Lys Arg Val Glu Ser Lys Tyr Gly Pro Pro |
| | Cys Pro Pro Cys Pro Ala Pro Pro Val Ala Gly Pro Ser Val |
| | Phe Leu Phe Pro Pro Lys Pro Lys Asp Thr Leu Met Ile Ser |
| | Arg Thr Pro Glu Val Thr Cys Val Val Val Asp Val Ser Gln |
| | Glu Asp Pro Glu Val Gln Phe Asn Trp Tyr Val Asp Gly Val |
| | Glu Val His Asn Ala Lys Thr Lys Pro Arg Glu Glu Gln Phe |
| | Asn Ser Thr Tyr Arg Val Val Ser Val Leu Thr Val Leu His |
| | Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala |
| | Lys Gly Gln Pro Arg Glu Pro Gln Val Tyr Thr Leu Pro Pro |
| | Ser Gln Glu Glu Met Thr Lys Asn Gln Val Ser Leu Thr Cys |
| | Leu Val Lys Gly Phe Tyr Pro Ser Asp Ile Ala Val Glu Trp |
| | Glu Ser Asn Gly Gln Pro Glu Asn Asn Tyr Lys Thr Thr Pro |
| | Pro Val Leu Asp Ser Asp Gly Ser Phe Phe Leu Tyr Ser Arg |
| | Leu Thr Val Asp Lys Ser Arg Trp Gln Glu Gly Asn Val Phe |
| | Ser Cys Ser Val Met His Glu Ala Leu His Asn Arg Phe Thr |
| | Gln Lys Ser Leu Ser Leu Ser Leu Gly Lys (SEQ ID NO: 79) |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric Fc regions, it is possible to incorporate a combination of the variant IgG4 Fc sequences set forth above, for example an Fc region comprising an Fc domain comprising the amino acid sequence of SEQ ID NO:30 of WO2014/121087 (or the bolded portion thereof) and an Fc domain comprising the amino acid sequence of SEQ ID NO:37 of WO2014/121087 (or the bolded portion thereof) or an Fc region comprising an Fc domain comprising the amino acid sequence of SEQ ID NO:31 of WO2014/121087 (or the bolded portion thereof) and an Fc domain comprising the amino acid sequence of SEQ ID NO:38 of WO2014/121087 (or the bolded portion thereof).

### 6.5.1.2. Fc Heterodimerization Variants

Certain multivalent anti-spike protein binding molecules entail dimerization between two Fc domains that, unlike a native immunoglobulin, are operably linked to non-identical N-terminal regions, e.g., one Fc domain connected to a Fab that binds to a first spike protein epitope and the other Fc domain connected to a different Fab that binds a second spike protein epitope. Inadequate heterodimerization of two Fc domains to form an Fc region can be an obstacle for increasing the yield of desired heterodimeric molecules and represents challenges for purification. A variety of approaches available in the art can be used in for enhancing dimerization of Fc domains that might be present in the ACE2 fusion proteins of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO 2009/089004A1.

The present disclosure provides multivalent anti-spike protein binding molecules comprising Fc heterodimers, i.e., Fc regions comprising heterologous, non-identical Fc domains. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired multivalent anti-spike protein binding molecule, while homodimerization of identical heavy chains will reduce yield of the desired multivalent anti-spike protein binding molecule. Thus, in a preferred embodiment, the polypeptides that associate to form a multivalent anti-spike protein binding molecule of the disclosure will contain CH3 domains with modifications that favor heterodimeric association relative to unmodified Fc domains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described e.g., in U.S. Pat. Nos. 5,731, 168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g., by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second Fc domains of the Fc region.

As an alternative, or in addition, to the use of Fc domains that are modified to promote heterodimerization, an Fc domain can be modified to allow a purification strategy that enables selections of Fc heterodimers. In one such embodiment, one polypeptide comprises a modified Fc domain that abrogates its binding to Protein A, thus enabling a purification method that yields a heterodimeric protein. See, for example, U.S. Pat. No. 8,586,713. As such, the IL 12 receptor agonists comprise a first CH3 domain and a second Ig CH3 domain, wherein the first and second Ig CH3 domains differ from one another by at least one amino acid, and wherein at least one amino acid difference reduces binding of the IL12 receptor agonist to Protein A as compared to a corresponding IL12 receptor agonist lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). This class of modifications is referred to herein as “star” mutations.

In some embodiments, the Fc can contain one or more mutations (e.g., knob and hole mutations) to facilitate heterodimerization as well as star mutations to facilitate purification.

### 6.6. Linkers

In certain aspects, the present disclosure provides multivalent anti-spike protein binding molecules in which two or more components are connected to one another by a peptide linker. By way of example and not limitation, linkers can be used to connect a spike protein ABD to a multimerization moiety.

A peptide linker can range from 1 amino acid to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 1 amino acid, at least 2 amino acids, at least 3 amino acids, at least 4 amino acids, at least 5 amino acids, at least 6 amino acids or at least 7 amino acids in length and optionally is up to 30 amino acids, up to 40 amino acids, up to 50 amino acids or up to 60 amino acids in length.

In some specific embodiments, a peptide linker ranges from 1 amino acid to 50 amino acids in length, e.g., ranges from 1 to 50, from 1 to 45, from 1 to 40, from 1 to 35, from 1 to 30, from 1 to 25, or from 1 to 20 amino acids in length. In other specific embodiments, a peptide linker ranges from 2 amino acids to 50 amino acids in length, e.g., ranges from 2 to 50, from 2 to 45, from 2 to 40, from 2 to 35, from 2 to 30, from 2 to 25, or from 2 to 20 amino acids in length. In some other specific embodiments, a peptide linker ranges from 3 amino acids to 50 amino acids in length, e.g., ranges from 3 to 50, from 3 to 45, from 3 to 40, from 3 to 35, from 3 to 30, from 3 to 25, or from 3 to 20 amino acids in length. In some other specific embodiments, a peptide linker ranges from 4 amino acids to 50 amino acids in length, e.g., ranges from 4 to 50, from 4 to 45, from 4 to 40, from 4 to 35, from 4 to 30, from 4 to 25, or from 4 to 20 amino acids in length. In some other specific embodiments, a peptide linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other specific embodiments, a peptide linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other specific embodiments, a peptide linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker is a G4S linker (SEQ ID NO:80). In some embodiments the linker comprises two consecutive G4S sequences (SEQ ID NO:81), three consecutive G4S sequences (SEQ ID NO:74), four consecutive G4S sequences (SEQ ID NO:82), five consecutive G4S sequences (SEQ ID NO:83), or six consecutive G4S sequences (SEQ ID NO:84).

### 6.6.1. Hinge Sequences

In other embodiments, the multivalent anti-spike protein binding molecules of the disclosure comprise a linker that is a hinge region. The hinge region can be a native or a modified hinge region. Hinge regions are typically found at the N-termini of Fc regions. The term “hinge region”, unless the context dictates otherwise, refers to a naturally or non-naturally occurring hinge sequence that in the context of a single or monomeric polypeptide chain is a monomeric hinge domain and in the context of a dimeric polypeptide (e.g., a homodimeric or heterodimeric multivalent anti-spike protein binding molecules formed by the association of two IgG Fc domains) can comprise two associated hinge sequences on separate polypeptide chains.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc domain or Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO 99/15549, WO 2005/003170, WO 2005/003169, WO 2005/003170, WO 98/25971 and WO 2005/003171 and these are incorporated herein by reference.

In some embodiments, a multivalent anti-spike protein binding molecule of the disclosure comprises an Fc region in which one or both Fc domains possesses an intact hinge region at its N-terminus.

In various embodiments, positions 233-236 within a hinge region may be G, G, G and unoccupied; G, G, unoccupied, and unoccupied; G, unoccupied, unoccupied, and unoccupied; or all unoccupied, with positions numbered by EU numbering.

In some embodiments, the multivalent anti-spike protein binding molecules of the disclosure comprise a modified hinge region that reduces binding affinity for an Fcγ receptor relative to a wild-type hinge region of the same isotype (e.g., human IgG1 or human IgG4).

In one embodiment, the multivalent anti-spike protein binding molecules of the disclosure comprise an Fc region in which each Fc domain possesses an intact hinge region at its N-terminus, where each Fc domain and hinge region is derived from IgG4, and each hinge region comprises the modified sequence CPPC (SEQ ID NO:85). The core hinge region of human IgG4 contains the sequence CPSC (SEQ ID NO:86) compared to IgG1 that contains the sequence CPPC (SEQ ID NO:85). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1): 105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 6.7. Nucleic Acids and Host Cells

In another aspect, the disclosure provides nucleic acids encoding multivalent anti-spike protein binding molecules of the disclosure. In some embodiments, the multivalent anti-spike protein binding molecules are encoded by a single nucleic acid. In other embodiments, the multivalent anti-spike protein binding molecules can be encoded by a plurality (e.g., two, three, four or more) nucleic acids.

A single nucleic acid can encode a multivalent anti-spike protein binding molecule that comprises a single polypeptide chain, a multivalent anti-spike protein binding molecule that comprises two or more polypeptide chains, or a portion of a multivalent anti-spike protein binding molecule that comprises more than two polypeptide chains (for example, a single nucleic acid can encode two polypeptide chains of a multivalent anti-spike protein binding molecule comprising three, four or more polypeptide chains, or three polypeptide chains of a multivalent anti-spike protein binding molecule comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, a multivalent anti-spike protein binding molecule comprising two or more polypeptide chains is encoded by two or more nucleic acids. The number of nucleic acids encoding a multivalent anti-spike protein binding molecule can be equal to or less than the number of polypeptide chains in the multivalent anti-spike protein binding molecule (for example, when two or more polypeptide chains are encoded by a single nucleic acid). The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.7.1. Vectors

The disclosure provides vectors comprising nucleotide sequences encoding a multivalent anti-spike protein binding molecule or a component thereof described herein, for example one or two of the polypeptide chains of a multivalent anti-spike protein binding molecule. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

### 6.7.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, Hela cells, COS cells, CHO cells, HEK293 cells, BHK cells and MDCKII cells. Suitable insect cells include, but are not limited to, Sf9 cells.

### 6.8. Pharmaceutical Compositions

The multivalent anti-spike protein binding molecules of the disclosure may be in the form of compositions comprising the multivalent anti-spike protein binding molecule and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the multivalent anti-spike protein binding molecules and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of a multivalent anti-spike protein binding molecule of the disclosure per dose. The quantity of a multivalent anti-spike protein binding molecule included in a unit dose will depend on the disease being treated, as well as other factors as are well known in the art. Such unit dosages may be in the form of a lyophilized dry powder containing an amount of multivalent anti-spike protein binding molecule suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of multivalent anti-spike protein binding molecule suitable for a single administration.

The pharmaceutical compositions may also be supplied in bulk from containing quantities of multivalent anti-spike protein binding molecules suitable for multiple administrations.

Pharmaceutical compositions may be prepared for storage as lyophilized formulations or aqueous solutions by mixing a multivalent anti-spike protein binding molecule having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of multivalent anti-spike protein binding molecule.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The multivalent anti-spike protein binding molecules of the disclosure can be formulated as pharmaceutical compositions comprising the multivalent anti-spike protein binding molecules, for example containing one or more pharmaceutically acceptable excipients or carriers. To prepare pharmaceutical or sterile compositions comprising the multivalent anti-spike protein binding molecules of the present disclosure, a multivalent anti-spike protein binding molecule preparation can be combined with one or more pharmaceutically acceptable excipient or carrier.

For example, formulations of multivalent anti-spike protein binding molecules can be prepared by mixing multivalent anti-spike protein binding molecules with physiologically acceptable carriers, excipients, or stabilizers in the form of, e.g., lyophilized powders, slurries, aqueous solutions, lotions, or suspensions (see, e.g., Hardman et al., 2001 , Goodman and Gilman's The Pharmacological Basis of Therapeutics, McGraw-Hill, New York, N.Y .; Gennaro, 2000, Remington: The Science and Practice of Pharmacy, Lippincott, Williams, and Wilkins, New York, N.Y .; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

### 6.9. Therapeutic Indications and Methods of Treatment

The present disclosure provides methods for using and applications for multivalent anti-spike protein binding molecules of the disclosure.

In certain aspects, the disclosure provides a method of preventing or treating a disease or condition in which an interaction between a RBD of a coronavirus and cellular ACE2 is implicated. In some embodiments, the disease or condition is prevented or treated by neutralization of the spike protein. In various embodiments, neutralization of the spike proteins comprises (a) inhibiting the ability of spike protein to bind to a receptor such as ACE2, (b) inhibiting cleavage of the spike protein by a protease such as TMPRSS2, (c) inhibiting the spike protein from mediating (i) viral entry into a host cell or (ii) viral reproduction in a host cell, or (d) any combination of two, three, or all four of (a), (b), (c)(i), and (c)(ii).

Accordingly, in some embodiments, the multivalent anti-spike protein binding molecules and pharmaceutical compositions of the disclosure can be used to inhibit an interaction between a RBD of a coronavirus and cellular ACE2. In some embodiments, the disclosure provides methods of inhibiting the interaction between the RBD of SARS-COV. In other embodiments, the disclosure provides methods of inhibiting the interaction between the RBD of SARS-COV-2. Accordingly, in some embodiments, the disclosure provides methods of inhibiting an interaction between a RBD of a coronavirus and cellular ACE2, comprising administering to a subject in need thereof a multivalent anti-spike protein binding molecule pharmaceutical composition as described herein.

In some embodiments, the disclosure provides methods of administrating a multivalent anti-spike protein binding molecule pharmaceutical composition as described herein to a subject who has been exposed to a coronavirus but is not diagnosed with an infection. In other embodiments, the subject has been tested positive for a coronavirus but is asymptomatic. In yet other embodiments, the subject has been tested positive for a coronavirus and is presymptomatic. In further embodiments, the subject has been tested positive for a coronavirus and is symptomatic. In other embodiments, the subject has developed COVID-19 or another coronavirus-mediated disease or condition.

In some embodiments, the disclosure provides a method of reducing the severity of coronavirus infection, comprising administering to a subject in need thereof the multivalent anti-spike protein binding molecule pharmaceutical composition as described herein.

In some other embodiments, the disclosure provides a method of reducing the viral load of a coronavirus, comprising administering to a subject in need thereof the multivalent anti-spike protein binding molecule pharmaceutical composition as described herein.

In further embodiments, the disclosure provides a method of preventing disease progression in a subject with a coronavirus infection, comprising administering to a subject in need thereof the multivalent anti-spike protein binding molecule pharmaceutical composition as described herein.

In some embodiments, the disclosure provides a method of reducing the duration of a coronavirus infection, comprising administering to a subject in need thereof the multivalent anti-spike protein binding molecule pharmaceutical composition as described herein.

In other embodiments, the disclosure provides a method of reducing the risk of severe disease or death in a subject with a coronavirus infection, comprising administering to a subject in need thereof the multivalent anti-spike protein binding molecule pharmaceutical composition as described herein.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

In the numbered embodiments that follow, the multimerization moieties are preferably derived from a mammalian multimerization moiety (e.g., a human Fc domain), the antigen binding domains are preferably from a human or humanized antibody, and the subjects are preferably mammals (e.g., humans).

1. A multivalent anti-spike protein binding molecule comprising at least 4 anti-spike protein antigen-binding domains (ABDs) operably linked by one or more multimerization moieties.

2 The multivalent anti-spike protein binding molecule of embodiment 1, which is tetravalent.

3. The multivalent anti-spike protein binding molecule of embodiment 1 or embodiment 2, wherein the antigen-binding domains (ABDs) are human or humanized.

4. The multivalent anti-spike protein binding molecule of any one of embodiments 1 to 3, wherein one or more (or all) ABDs comprise CDR sequences set forth in any one of Tables 1-3.

5. The multivalent anti-spike protein binding molecule of any one of embodiments 1 to 3, wherein one or more (or all) ABDs comprise CDR sequences set forth in any one of Tables 1-4.

6. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 1.

7. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 2.

8. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 3.

9. The multivalent anti-spike protein binding molecule of any one of embodiments 1 to 5, wherein one or more (or all) ABDs comprise the CDR-H1, CDR-H2, CDR-H3, CDR-L1, CDR-L2, and CDR-L3 sequences of an antibody set forth in Table 4.

10. The multivalent anti-spike protein binding molecule of any one of embodiments 1 to 5, wherein one or more (or all) ABDs comprise:
(a) a VH comprising CDR-H1, CDR-H2, and CDR-H3 having the amino acid sequences of SEQ ID NOs: 579, 580, and 581, respectively; and
(b) a VL comprising CDR-L1, CDR-L2, and CDR-L3 having the amino acid sequences of SEQ ID NOs: 398, 372, and 583, respectively.

11. The multivalent anti-spike protein binding molecule of any one of embodiments 1 to 5, wherein one or more (or all) ABDs comprise:
(a) a VH comprising CDR-H1, CDR-H2, and CDR-H3 having the amino acid sequences of SEQ ID NOs: 507, 508, and 509, respectively; and
(b) a VL comprising CDR-L1, CDR-L2, and CDR-L3 having the amino acid sequences of SEQ ID NOs: 511, 407, and 512, respectively.

12. The multivalent anti-spike protein binding molecule of any one of embodiments 1 to 5, wherein one or more (or all) ABDs comprise:
(a) a VH comprising CDR-H1, CDR-H2, and CDR-H3 having the amino acid sequences of SEQ ID NOs: 450, 451, and 452, respectively; and
(b) a VL comprising CDR-L1, CDR-L2, and CDR-L3 having the amino acid sequences of SEQ ID NOs: 454, 415, and 455, respectively.

13. The multivalent anti-spike protein binding molecule of any one of embodiments 1 to 3, wherein one or more (or all) ABDs comprise VH and VL sequences set forth in any one of Tables 1-3.

14. The multivalent anti-spike protein binding molecule of any one of embodiments 1 to 3, wherein one or more (or all) ABDs comprise VH and VL sequences set forth in any one of Tables 1-4.

15. The multivalent anti-spike protein binding molecule of any one of embodiments 1 to 5, wherein one or more (or all) ABDs comprise VH and VL sequences of an antibody set forth in Table 1.

16. The multivalent anti-spike protein binding molecule of any one of embodiments 1 to 5, wherein one or more (or all) ABDs comprise VH and VL sequences of an antibody set forth in Table 2.

17. The multivalent anti-spike protein binding molecule of any one of embodiments 1 to 5, wherein one or more (or all) ABDs comprise VH and VL sequences of an antibody set forth in Table 3.

18. The multivalent anti-spike protein binding molecule of any one of embodiments 1 to 5, wherein one or more (or all) ABDs comprise VH and VL sequences of an antibody set forth in Table 4.

19. The multivalent anti-spike protein binding molecule of any one of embodiments 1 to 5, wherein one or more (or all) ABDs comprise (a) a VH having at least 90%, at least 95%, at least 96%, at least 97%, at least 98%, at least 99%, or 100% sequence identity to the amino acid sequence of SEQ ID NO:578 and (b) a VL having at least 90%, at least 95%, at least 96%, at least 97%, at least 98%, at least 99%, or 100% sequence identity to the amino acid sequence of SEQ ID NO:582.

20. The multivalent anti-spike protein binding molecule of any one of embodiments 1 to 5, wherein one or more (or all) ABDs comprise (a) a VH comprising the amino acid sequence of SEQ ID NO:578 and (b) a VL comprising the amino acid sequence of SEQ ID NO:582.

21. The multivalent anti-spike protein binding molecule of any one of embodiments 1 to 5, wherein one or more (or all) ABDs comprise (a) a VH having at least 90%, at least 95%, at least 96%, at least 97%, at least 98%, at least 99%, or 100% sequence identity to the amino acid sequence of SEQ ID NO:506 and (b) a VL having at least 90%, at least 95%, at least 96%, at least 97%, at least 98%, at least 99%, or 100% sequence identity to the amino acid sequence of SEQ ID NO:510.

22. The multivalent anti-spike protein binding molecule of any one of embodiments 1 to 5, wherein one or more (or all) ABDs comprise (a) a VH comprising the amino acid sequence of SEQ ID NO:506 and (b) a VL comprising the amino acid sequence of SEQ ID NO:510.

23. The multivalent anti-spike protein binding molecule of any one of embodiments 1 to 5, wherein one or more (or all) ABDs comprise (a) a VH having at least 90%, at least 95%, at least 96%, at least 97%, at least 98%, at least 99%, or 100% sequence identity to the amino acid sequence of SEQ ID NO:449 and (b) a VL having at least 90%, at least 95%, at least 96%, at least 97%, at least 98%, at least 99%, or 100% sequence identity to the amino acid sequence of SEQ ID NO:453.

24. The multivalent anti-spike protein binding molecule of any one of embodiments 1 to 5, wherein one or more (or all) ABDs comprise (a) a VH comprising the amino acid sequence of SEQ ID NO:449 and (b) a VL comprising the amino acid sequence of SEQ ID NO:453.

25. The multivalent anti-spike protein binding molecule of any one of embodiments 1 to 24, wherein one or more (or all) ABDs are neutralizing.

26. The multivalent anti-spike protein binding molecule of any one of embodiments 1 to 25, which is capable of neutralizing the SARS-COV-2 variant BA. 1.

27. The multivalent anti-spike protein binding molecule of any one of embodiments 1 to 25, which is capable of neutralizing the SARS-COV-2 variant BA.2.

28. The multivalent anti-spike protein binding molecule of any one of embodiments 1 to 27, wherein the one or more multimerization moieties are Fc domains.

29. The multivalent anti-spike protein binding molecule of any one of embodiments 1 to 28, which comprises two half antibodies, each comprising an Fc domain.

30. The multivalent anti-spike protein binding molecule of embodiment 28 or embodiment 29, wherein the Fc domains are IgG domains.

31. The multivalent anti-spike protein binding molecule of embodiment 30, wherein the IgG domains are IgG1 domains.

32. The multivalent anti-spike protein binding molecule of embodiment 30, wherein the IgG domains are IgG4 domains.

33. The multivalent anti-spike protein binding molecule of any one of embodiments 29 to 32, wherein each half antibody comprises two ABDs.

34. The multivalent anti-spike protein binding molecule of any one of embodiments 1 to 33, comprising:
(a) a first half antibody comprising:
   (i) a first ABD, typically comprising a first VH and a first VL;
   (ii) an optional first linker;
   (iii) a second ABD, typically comprising a second VH and a second VL;
   (iv) an optional first hinge domain; and
   (v) a first Fc domain; and
(b) a second half antibody comprising:
   (i) a third ABD, typically comprising a third VH and a third VL;
   (ii) an optional second linker;
   (iii) a fourth ABD, typically comprising a fourth VH and a fourth VL;
   (iv) an optional second hinge domain; and
   (v) a second Fc domain.

35. The multivalent anti-spike protein binding molecule of any one of embodiments 1 to 33:
(a) a first half antibody comprising:
   (i) a first ABD, typically comprising a first VH and a first VL;
   (ii) an optional first hinge domain;
   (iii) a first Fc domain;
   (iv) an optional first linker; and
   (v) a second ABD, typically comprising a second VH and a second VL; and
(b) a second half antibody comprising:
   (i) a third ABD, typically comprising a third VH and a third VL;
   (ii) an optional second hinge domain;
   (iii) a second Fc domain;
   (iv) an optional second linker; and
   (v) a fourth ABD, typically comprising a fourth VH and a fourth VL.

36. The multivalent anti-spike protein binding molecule of embodiment 34 or embodiment 35, wherein the first Fc domain and the second Fc domain form an Fc homodimer.

37. The multivalent anti-spike protein binding molecule of embodiment 34 or embodiment 35, wherein the first Fc domain and the second Fc domain form an Fc heterodimer.

38. The multivalent anti-spike protein binding molecule of embodiment 37, wherein the first Fc domain or the second Fc domain comprises knob mutations and the other Fc domain comprises hole mutations.

39. The multivalent anti-spike protein binding molecule of any one of embodiments 34 to 38, wherein the first Fc domain or the second Fc domain comprises a star mutation.

40. The multivalent anti-spike protein binding molecule of any one of embodiments 34 to 39, which comprises a first hinge domain and a second hinge domain.

41. The multivalent anti-spike protein binding molecule of embodiment 40, wherein the first hinge domain and the second hinge domain are IgG1 hinge domains.

42. The multivalent anti-spike protein binding molecule of embodiment 40, wherein the first hinge domain and the second hinge domain are IgG4 hinge domains.

43. The multivalent anti-spike protein binding molecule of embodiment 40, wherein the first hinge domain and the second hinge domain are chimeric hinge domains.

44. The multivalent anti-spike protein binding molecule of any one of claims 34 to 43, which lacks the first linker and the second linker.

45. The multivalent anti-spike protein binding molecule of any one of embodiments 34 to 43, which comprises the first linker and the second linker.

46. The multivalent anti-spike protein binding molecule of embodiment 45, wherein the first linker and second linker are each independently selected from (a) 1-60 amino acids in length, or (b) 1-40 amino acids in length, or (c) any range or value of linker length set forth in Section 6.6.

47. The multivalent anti-spike protein binding molecule of embodiment 45 or embodiment 46, wherein the first linker and second linker each comprises a glycine-serine sequence, optionally wherein the glycine-serine sequence is (GₙS)ₓ (SEQ ID NO:87), wherein n=0-5 and x=1-6.

48. The multivalent anti-spike protein binding molecule of any one of embodiments 34 to 47, wherein the first, second, third and fourth ABDs are the same.

49. The multivalent anti-spike protein binding molecule of any one of embodiments 34 to 48, wherein the first and second ABDs are the same.

50. The multivalent anti-spike protein binding molecule of embodiment 49, wherein the third and fourth ABDs are the same.

51. The multivalent anti-spike protein binding molecule of embodiment 50, wherein the third and fourth ABDs are different from the first and second ABDs.

52. The multivalent anti-spike protein binding molecule of any one of embodiments 34 to 47, wherein the first and third ABDs are the same.

53. The multivalent anti-spike protein binding molecule of embodiment 52, wherein the second and fourth ABDs are the same.

54. The multivalent anti-spike protein binding molecule of embodiment 53, wherein the second and fourth ABDs are different from the first and third ABDs.

55. The multivalent anti-spike protein binding molecule of any one of embodiments 1 to 54, in which at least two of the ABDs are Fab domains, optionally wherein the Fab domains are not single chain Fab domains.

56. The multivalent anti-spike protein binding molecule of any one of embodiments 1 to 54, in which all the ABDs are Fab domains, optionally wherein the Fab domains are not single chain Fab domains.

57. The multivalent anti-spike protein binding molecule of any one of embodiments 1 to 54, in which at least two of the ABDs are scFvs.

58. The multivalent anti-spike protein binding molecule of any one of embodiments 1 to 54, in which all of the ABDs are scFvs.

59. The multivalent anti-spike protein binding molecule of any one of embodiments 1 to 58 which is monospecific.

60. The multivalent anti-spike protein binding molecule of any one of embodiments 1 to 58, which is multispecific.

61. The multivalent anti-spike protein binding molecule of embodiment 60, which is bispecific.

62. A multivalent anti-spike protein binding molecule comprising at least 4 means for binding spike protein operably linked by one or more multimerization moieties.

63. The multivalent anti-spike protein binding molecule of embodiment 62, which is tetravalent for the means for binding spike protein.

64. The multivalent anti-spike protein binding molecule of embodiment 62 or 63, which is capable of neutralizing the SARS-COV-2 variant BA.1.

65. The multivalent anti-spike protein binding molecule of embodiment 62 or 63, which is capable of neutralizing the SARS-COV-2 variant BA.2.

66. The multivalent anti-spike protein binding molecule of any one of embodiments 62 to 65, wherein the one or more multimerization moieties are Fc domains.

67. The multivalent anti-spike protein binding molecule of any one of embodiments 62 to 65, which comprises two half antibodies, each comprising an Fc domain.

68. The multivalent anti-spike protein binding molecule of embodiment 66 or embodiment 67, wherein the Fc domains are IgG domains.

69. The multivalent anti-spike protein binding molecule of embodiment 68, wherein the IgG domains are IgG1 domains.

70. The multivalent anti-spike protein binding molecule of embodiment 68, wherein the IgG domains are IgG4 domains.

71. The multivalent anti-spike protein binding molecule of any one of embodiments 67 to 70, wherein each half antibody comprises two means for binding spike protein.

72. The multivalent anti-spike protein binding molecule of any one of embodiments 1 to 71, comprising:
(a) a first half antibody comprising:
   (i) a first means for binding spike protein;
   (ii) an optional first linker;
   (iii) a second means for binding spike protein;
   (iv) an optional first hinge domain; and
   (v) a first Fc domain; and
(b) a second half antibody comprising:
   (i) a third means for binding spike protein;
   (ii) an optional second linker;
   (iii) a fourth means for binding spike protein;
   (iv) an optional second hinge domain; and
   (v) a second Fc domain.

73. The multivalent anti-spike protein binding molecule of any one of embodiments 1 to 71:
(a) a first half antibody comprising:
   (i) a first means for binding spike protein;
   (ii) an optional first hinge domain;
   (iii) a first Fc domain;
   (iv) an optional first linker; and
   (v) a second means for binding spike protein; and
(b) a second half antibody comprising:
   (i) a third means for binding spike protein;
   (ii) an optional second hinge domain;
   (iii) a second Fc domain;
   (iv) an optional second linker; and
   (v) a fourth means for binding spike protein.

74. The multivalent anti-spike protein binding molecule of embodiment 72 or embodiment 73, wherein the first Fc domain and the second Fc domain form an Fc homodimer.

75. The multivalent anti-spike protein binding molecule of embodiment 72 or embodiment 73, wherein the first Fc domain and the second Fc domain form an Fc heterodimer.

76. The multivalent anti-spike protein binding molecule of embodiment 75, wherein the first Fc domain or the second Fc domain comprises knob mutations and the other Fc domain comprises hole mutations.

77. The multivalent anti-spike protein binding molecule of any one of embodiments 72 to 76, wherein the first Fc domain or the second Fc domain comprises a star mutation.

78. The multivalent anti-spike protein binding molecule of any one of embodiments 72 to 77, which comprises a first hinge domain and a second hinge domain.

79. The multivalent anti-spike protein binding molecule of embodiment 78, wherein the first hinge domain and the second hinge domain are IgG1 hinge domains.

80. The multivalent anti-spike protein binding molecule of embodiment 78, wherein the first hinge domain and the second hinge domain are IgG4 hinge domains.

81. The multivalent anti-spike protein binding molecule of embodiment 78, wherein the first hinge domain and the second hinge domain are chimeric hinge domains.

82. The multivalent anti-spike protein binding molecule of any one of claims 72 to 81, which lacks the first linker and the second linker.

83. The multivalent anti-spike protein binding molecule of any one of embodiments 72 to 81, which comprises the first linker and the second linker.

84. The multivalent anti-spike protein binding molecule of embodiment 83, wherein the first linker and second linker are each independently selected from (a) 1-60 amino acids in length, or (b) 1-40 amino acids in length, or (c) any range or value of linker length set forth in Section 6.6.

85. The multivalent anti-spike protein binding molecule of embodiment 83 or embodiment 84, wherein the first linker and second linker each comprises a glycine-serine sequence, optionally wherein the glycine-serine sequence is (GₙS)ₓ (SEQ ID NO:87), wherein n=0-5 and x=1-6.

86. The multivalent anti-spike protein binding molecule of any one of embodiments 62 to 85, wherein the first half antibody comprises a first Fab comprising the first means for binding spike protein.

87. The multivalent anti-spike protein binding molecule of any one of embodiments 62 to 85, wherein the first half antibody comprises a first scFv comprising the first means for binding spike protein.

88. The multivalent anti-spike protein binding molecule of any one of embodiments 62 to 87, wherein the first half antibody comprises a second Fab comprising the second means for binding spike protein.

89. The multivalent anti-spike protein binding molecule of any one of embodiments 62 to 87, wherein the first half antibody comprises a second scFv comprising the second means for binding spike protein.

90. The multivalent anti-spike protein binding molecule of any one of embodiments 62 to 89, wherein the second half antibody comprises a third Fab comprising the third means for binding spike protein.

91. The multivalent anti-spike protein binding molecule of any one of embodiments 62 to 89, wherein the second half antibody comprises a third scFv comprising the third means for binding spike protein.

92. The multivalent anti-spike protein binding molecule of any one of embodiments 62 to 91, wherein the second half antibody comprises a fourth scFv comprising the fourth means for binding spike protein.

93. The multivalent anti-spike protein binding molecule of any one of embodiments 62 to 91, wherein the second half antibody comprises a fourth scFv comprising the fourth means for binding spike protein.

94. The multivalent anti-spike protein binding molecule of any one of embodiments 62 to 93 which is monospecific.

95. The multivalent anti-spike protein binding molecule of any one of embodiments 62 to 93, which is multispecific.

96. The multivalent anti-spike protein binding molecule of embodiment 95, which is bispecific.

97. A nucleic acid or plurality of nucleic acids encoding the multivalent anti-spike protein binding molecule of any one of embodiments 1 to 96.

98. A host cell engineered to express the multivalent anti-spike protein binding molecule of any one of embodiments 1 to 96 or the nucleic acid(s) of embodiment 97.

99. A method of producing the multivalent anti-spike protein binding molecule of any one of embodiments 1 to 96, comprising culturing the host cell of embodiment 98 and recovering the multivalent anti-spike protein binding molecule expressed thereby.

100. A pharmaceutical composition comprising the multivalent anti-spike protein binding molecule of any one of embodiments 1 to 96 and an excipient.

101. A method of treating a coronavirus disease, comprising administering to a subject in need thereof the multivalent anti-spike protein binding molecule of any one of embodiments 1 to 96 or the pharmaceutical composition of embodiment 100.

102. A method of inhibiting an interaction between a RBD of a coronavirus and cellular ACE2, comprising administering to a subject in need thereof the multivalent anti-spike protein binding molecule of any one of embodiments 1 to 96 or the pharmaceutical composition of embodiment 100.

103. A method of neutralizing a coronavirus spike protein in vivo, comprising administering to a subject in need thereof the multivalent anti-spike protein binding molecule of any one of embodiments 1 to 96 or the pharmaceutical composition of embodiment 100.

104. A method of inhibiting protease-mediated cleavage (e.g., TMPRSS2-mediated cleavage) of a coronavirus spike protein in vivo, comprising administering to a subject in need thereof the multivalent anti-spike protein binding molecule of any one of embodiments 1 to 96 or the pharmaceutical composition of embodiment 100.

105. A method of inhibiting viral entry of a coronavirus into a host cell in a subject, comprising administering to a subject in need thereof the multivalent anti-spike protein binding molecule of any one of embodiments 1 to 96 or the pharmaceutical composition of embodiment 100.

106. A method of inhibiting reproduction of a coronavirus spike protein in a host cell in a subject, comprising administering to a subject in need thereof the multivalent anti-spike protein binding molecule of any one of embodiments 1 to 96 or the pharmaceutical composition of embodiment 100.

107. A method reducing the severity of coronavirus infection, comprising administering to a subject in need thereof the multivalent anti-spike protein binding molecule of any one of embodiments 1 to 96 or the pharmaceutical composition of embodiment 100.

108. A method of reducing the viral load of a coronavirus, comprising administering to a subject in need thereof the multivalent anti-spike protein binding molecule of any one of embodiments 1 to 96 or the pharmaceutical composition of embodiment 100.

109. A method of preventing disease progression in a subject with a coronavirus infection, comprising administering to a subject in need thereof the multivalent anti-spike protein binding molecule of any one of embodiments 1 to 96 or the pharmaceutical composition of embodiment 100.

110. A method of reducing the duration of a coronavirus infection, comprising administering to a subject in need thereof the multivalent anti-spike protein binding molecule of any one of embodiments 1 to 96 or the pharmaceutical composition of embodiment 100.

111. A method of reducing the risk of severe disease or death in a subject with a coronavirus infection, comprising administering to a subject in need thereof the multivalent anti-spike protein binding molecule of any one of embodiments 1 to 96 or the pharmaceutical composition of embodiment 100.

112. The method of any one of embodiments 101 to 111, wherein the coronavirus is SARS-COV.

113. The method of any one of embodiments 101 to 111, wherein the coronavirus is SARS-COV-2.

### 8. EXAMPLES

### 8.1. Materials and Methods

### 8.1.1. Construction and Production of Tetravalent Fab-Fc AF Antibodies

Both monospecific and bispecific tetravalent 2×2 N-Fab AF antibodies were constructed by connecting two identical VH-CH1 fragments linked via (G4S)×3 linker (SEQ ID NO:74) to the N-terminal end of an Fc fragment via the hinge region (FIGS. 1A and 1B). 2×2 C-Fab AF antibodies were constructed by connecting a VH-CH1 fragment to the N-terminal end of an Fc fragment, which in turn is linked to another identical VH-CH1 fragment on its C-terminal end via a (G4S)×3 linker (SEQ ID NO:74) (FIGS. 1C and 1D). To achieve consistency in production, all AF antibodies were incorporated with “knob-in-hole” mutations in the Fc region to promote Fc heterodimer formation a follow-up step of Red-Ox annealing assembly.

All antibodies were separately expressed in Expi293™ cells (ThermoFisher) as half-antibodies, termed as “knob chains” and “hole* chains” by transient transfection, following the manufacturer's protocol. Antibodies were purified from the supernatant using Hitrap Protein G HP (Cytiva). After single step elution, the antibodies were neutralized, dialyzed into a final buffer of phosphate buffered saline (PBS) with 5% glycerol, aliquoted and stored at −80° C.

For Red-Ox annealing assembly, a solution was made containing 0.5 mg/mL knob chains, 0.5 mg/mL hole* chains, 50 mM Tris pH 8.0, 25 mM 2-MEA, and 50 mM L-arginine. The reaction was performed at 37° C. for 5 hours, and then at 4° C. overnight. The product was desalted into a final buffer of phosphate buffered saline (PBS) with 5% glycerol using Zeba Spin Desalting Columns (ThermoFisher), aliquoted and stored at −80° C.

The elution fraction material was further polished to increase the purity of the species of interest by SEC. Hence, a Superdex 200 10/300GL column (Cytiva) was employed at a flow rate of 0.75 mL/min, in 1xDPBS, 5% Glycerol, pH7.4 running buffer. Fractions of interested were pooled and concentrated.

### 8.1.2. SARS-COV-2 Pseudovirus/Variant Neutralization Assay

Vero cells were cultured in DMEM high glucose medium with sodium pyruvate and without glutamine, supplemented with 10% heat-inactivated FBS and Penicillin/Streptomycin/L-glutamine (Complete DMEM) and seeded at 20,000 cells/well in 96-well black/clear bottom cell culture plates. On the day of the assay, the antibodies were diluted to 2X assay concentration and serially diluted 3-fold, for a total of 11 concentrations (e.g., 40 nM to 677.4 fM for IgG controls and all constructs in Table 10. Concentrations used for constructs in Table 9 were 13.3 nM to 225.8 fM). All dilutions were performed using infection media consisting of DMEM high glucose medium without sodium pyruvate/with glutamine that was supplemented with Sodium Pyruvate, 0.2% IgG-free BSA, and Gentamicin.

The pVSV-Luc-SARS-COV2-S pseudoviruses used herein were non-replicating VSV-DG, that expressed a dual GFP/firefly luciferase reporter in place of its native glycoprotein, and pseudotyped with SARS-COV-2 Spike. The SARS-COV-2 pseudoviruses or variants were diluted 1:4 in infection media, then combined 1:1 with antibody dilutions for a final pseudovirus/variant dilution of 1:8 and final test article concentrations of 20 nM to 338.7 fM for all IgG controls and constructs shown in Table 10, or 6.7 nM to 112.9 fM for constructs shown in Table 9 and incubated at room temperature for 30 minutes. Next, the culture media were removed from the cells and the combined antibodies and pseudoviruses/variants were added 100 uL/well in duplicates to the wells, which were then incubated at 37C, 5% CO2 for 24 hours. At 24 hours post-infection, media were removed from the wells, and the cells were lysed using 100 μL/well Glo-Lysis buffer (Promega). Immediately before reading luminescence on the Spectramax i3X plate reader, 100 uL prepared Bright-Glo substrate (Promega) was added to the lysates. The results were exported to Microsoft Excel, where % neutralization was calculated with the following equation: % Neutralization=((1−well value−medium control)/(virus control−medium control))×100% Neutralization is then plotted in GraphPad Prism and analyzed using nonlinear regression: log(inhibitor) vs. response-Variable slope (four parameter) to calculate IC50 values.

### 8.2. Example 1: Neutralization Activity of Tetravalent 2×2 N-Fab AFs

Using five different Fab moieties against SARS-COV-2 S protein (REGN10933, 10985, 10987, 14256, and 14315; comprising VH and VL domains from mAb10933, mAb10985, mAb10987, mAb14256, and mAb14315, as set forth in Tables 3 and 4, respectively), a total of five monospecific and 10 bispecific 2×2 N-Fab AF constructs were generated as described in Section 8.1.1 and are listed in Table 7. Cell cultures and virus neutralization assays were conducted as described in Section 8.1.2.

TABLE 7
Exemplary Tetravalent 2 × 2 N-Fab AF Constructs

| Construct Name | Fab Specificity | Knob Fab | Hole* Fab |
|---|---|---|---|
| COVAF-16 | Monospecific | REGN10933 | REGN10933 |
| COVAF-17 | Bispecific | REGN10933 | REGN14256 |
| COVAF-18 | Bispecific | REGN10987 | REGN10933 |
| COVAF-19 | Bispecific | REGN10933 | REGN14315 |
| COVAF-20 | Bispecific | REGN10985 | REGN10933 |
| COVAF-21 | Monospecific | REGN14256 | REGN14256 |
| COVAF-22 | Bispecific | REGN14256 | REGN10987 |
| COVAF-23 | Bispecific | REGN14315 | REGN14256 |
| COVAF-24 | Bispecific | REGN14256 | REGN10985 |
| COVAF-25 | Monospecific | REGN10987 | REGN10987 |
| COVAF-26 | Bispecific | REGN10987 | REGN14315 |
| COVAF-27 | Bispecific | REGN10985 | REGN10987 |
| COVAF-28 | Monospecific | REGN14315 | REGN14315 |
| COVAF-29 | Bispecific | REGN14315 | REGN10985 |
| COVAF-30 | Monospecific | REGN10985 | REGN10985 |
| COVAF-46 | Bispecific | REGN10933 | REGN14287 |
| COVAF-47 | Bispecific | REGN14287 | REGN14256 |
| COVAF-48 | Bispecific | REGN10987 | REGN14287 |
| COVAF-49 | Bispecific | REGN14287 | REGN14315 |
| COVAF-50 | Bispecific | REGN10985 | REGN14287 |
| COVAF-51 | Monospecific | REGN14287 | REGN14287 |

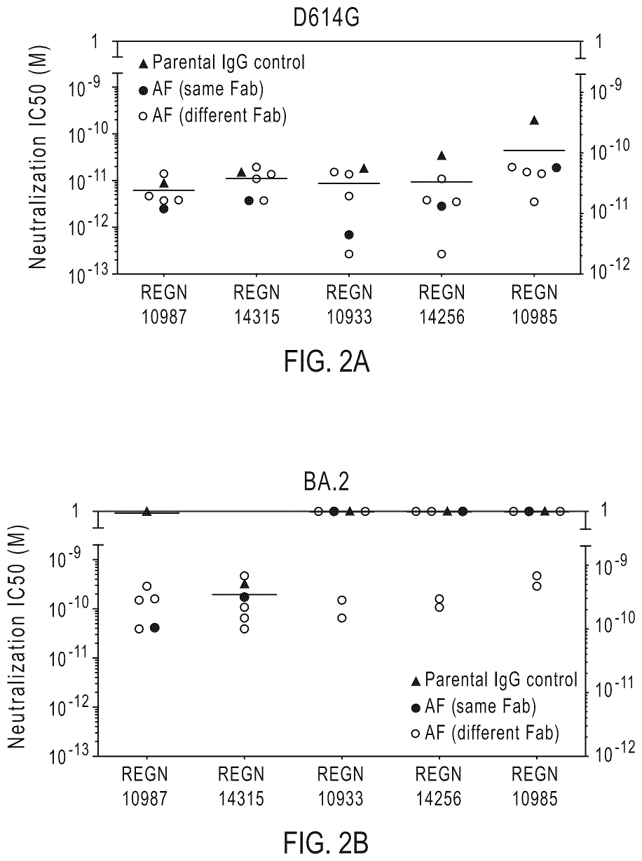

SARS-COV-2 pseudovirus or variant neutralization IC50 values of 2×2 N-Fab AF constructs were compared to the IC50 values of the IgG mAb controls with the same Fab moiety. In general, monospecific 2×2 N-Fab AF constructs were more effective at neutralizing the SARS-COV-2 pseudovirus, D614G, than the IgG mAb controls (FIG. 2A). This observation suggests that an increase in valency can help boost neutralization activities. Most bispecific 2×2 N-Fab AF constructs also performed better than their IgG mAb controls. Yet, the change in neutralization activity depended on which Fab arms were used to generate the constructs.

The ability of monospecific and bispecific anti-SARS-COV-2 2×2 N-Fab constructs to neutralize the SARS-COV-2 BA.2 variant entirely depended on which Fab arms were used to generate the constructs. For instance, constructs without the Fab moieties REGN14315 and REGN10987 failed to neutralize the BA.2 variant. However, the inclusion of either REGN14315 or REGN10987 in the construct enabled the constructs to neutralize the BA.2 variant (FIG. 2B).

### 8.3. Example 2: Neutralization Activity of Tetravalent 2×2 C-Fab AFs

As was done in example 1, the same five Fab moieties were used to generate a total of five monospecific and 10 bispecific 2×2 C-Fab AF constructs as described in Section 8.1.1 and are listed in Table 8. Cell cultures and virus neutralization assays were conducted as described in Section 8.1.2.

TABLE 8
Exemplary Tetravalent 2 × 2 C-Fab AF Constructs

| Construct Name | Fab Specificity | Knob Fab | Hole* Fab |
|---|---|---|---|
| COVAF-31 | Monospecific | REGN10933 | REGN10933 |
| COVAF-32 | Bispecific | REGN10933 | REGN14256 |
| COVAF-33 | Bispecific | REGN10987 | REGN10933 |
| COVAF-34 | Bispecific | REGN10933 | REGN14315 |
| COVAF-35 | Bispecific | REGN10985 | REGN10933 |
| COVAF-36 | Monospecific | REGN14256 | REGN14256 |
| COVAF-37 | Bispecific | REGN14256 | REGN10987 |
| COVAF-38 | Bispecific | REGN14315 | REGN14256 |
| COVAF-39 | Bispecific | REGN14256 | REGN10985 |
| COVAF-40 | Monospecific | REGN10987 | REGN10987 |
| COVAF-41 | Bispecific | REGN10987 | REGN14315 |
| COVAF-42 | Bispecific | REGN10985 | REGN10987 |
| COVAF-43 | Monospecific | REGN14315 | REGN14315 |
| COVAF-44 | Bispecific | REGN14315 | REGN10985 |
| COVAF-45 | Monospecific | REGN10985 | REGN10985 |
| COVAF-52 | Bispecific | REGN10933 | REGN14287 |
| COVAF-53 | Bispecific | REGN14287 | REGN14256 |
| COVAF-54 | Bispecific | REGN10987 | REGN14287 |
| COVAF-55 | Bispecific | REGN14287 | REGN14315 |
| COVAF-56 | Bispecific | REGN10985 | REGN14287 |
| COVAF-57 | Monospecific | REGN14287 | REGN14287 |

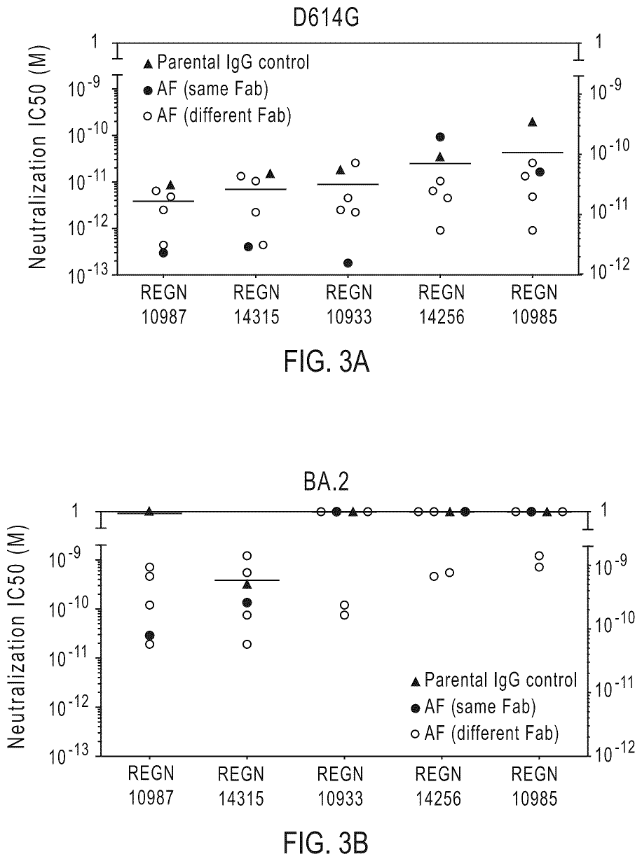

SARS-COV-2 pseudovirus or variant neutralization IC50 values of 2×2 C-Fab AF constructs were compared to the IC50 values of the IgG mAb controls with the same Fab moiety. In general, monospecific 2×2 C-Fab AF constructs were more effective at neutralizing the SARS-COV-2 pseudovirus than the IgG mAb controls except for COVAF36, which has the Fab moiety REGN14356 (FIG. 3A). As it was the case in example 1, most bispecific 2×2 C-Fab AF constructs also performed better than their IgG mAb controls. Again, the change in neutralization activity depended on which Fab arms were used to generate the constructs.

The ability of monospecific and bispecific 2×2 C-Fab constructs to neutralize the SARS-CoV-2 BA.2 variant again depended entirely on which Fab arms were used to generate the constructs. Consistent with the observations in example 1, constructs without the Fab moieties REGN14315 and REGN10987 failed to neutralize the BA.2 variant. Nevertheless, in accordance with 2×2 N-Fab screening, the inclusion of either REGN14315 or REGN10987 in the construct enabled the 2×2 C-Fab AF constructs to neutralize the BA.2 variant (FIG. 3B).

Three selected 2×2 C-Fab leads, COVAF-40, 41 and 43, were further purified using size-exclusion chromatography and tested potency and breadth coverage in SARS-COV2 SARS-CoV-2 pseudovirus neutralization assay using multiple Omicron variants including: BA.1, BA.2, BA.2.12.1, BA.4/BA.5 and BA.4+BA.4.6 (Table 9). In comparison to parental IgG control, 2×2 C-Fab COV-AF40 (10987×10987) demonstrated enhanced broad neutralization potency across D614G, and all Omicron variants tested over REGN10987 IgG. However, this enhancement is not sufficient to provide protection against individual Omicron variant as a range of 18-576-fold loss of potency is seen when compared with neutralization potency (IC50) for REGN10987 against D614G (Table 9).

TABLE 9
Summary of Pseudovirus Neutralization Activity of Top 2 ×
2 C-Fab AFs Against D614G and Multiple Omicron Variants

| | | | | IC50 (M) | | | | | |
|---|---|---|---|---|---|---|---|---|---|
| | | | | | | | | | BA.4 + |
| | | REGN | MW | | | | | BA.4/ | R346T |
| Format | AF ID | arms | (kDa) | D614G | BA.1 | BA.2 | BA.2.12.1 | BA.5 | (BA.4.6) |
| 2 × 2 | COVAF-40 | 10987 × | 250 | 2.73E−12 | 3.05E−09 | 9.38E−11 | 2.01E−10 | 2.40E−10 | 3.17E−10 |
| C-Fab | | 10987 | | | | | | | |
| | COVAF-43 | 14315 × | 250 | 1.05E−11 | 3.37E−11 | 1.84E−10 | 1.97E−10 | 1.35E−10 | 2.66E−09 |
| | | 14315 | | | | | | | |
| | COVAF-41 | 10987 × | 250 | 8.48E−12 | 4.34E−10 | 6.79E−11 | 5.86E−11 | 8.82E−11 | 1.12E−09 |
| | | 14315 | | | | | | | |
| Ctrl | REGN10987 | 10987 | 150 | | NC | 1.06E−09 | 1.48E−09 | 9.33E−10 | 1.25E−09 |
| IgG | REGN14315 | 14315 | 150 | 1.15E−12 | 5.47E−12 | 3.59E−11 | 6.73E−11 | 4.97E−11 | 1.35E−09 |
| | REGN14315 + | 14315 + | 150 | 3.68E−12 | 1.69E−11 | 6.99E−11 | 1.11E−10 | 1.10E−10 | 6.98E−10 |
| | REGN10987 | 10987 | | | | | | | |

| | | | | Fold change over R10987 against D614G | | | | | |
|---|---|---|---|---|---|---|---|---|---|
| | | | | | | | | | BA.4 + |
| | | REGN | MW | | | | | BA.4/ | R346T |
| Format | AF ID | arms | (kDa) | D614G | BA.1 | BA.2 | BA.2.12.1 | BA.5 | (BA.4.6) |
| 2 × 2 | COVAF-40 | 10987 × | 250 | 0.51 | 575.58 | 17.69 | 37.88 | 45.22 | 59.77 |
| C-Fab | | 10987 | | | | | | | |
| | COVAF-43 | 14315 × | 250 | 1.98 | 6.35 | 34.63 | 37.21 | 25.44 | 502.36 |
| | | 14315 | | | | | | | |
| | COVAF-41 | 10987 × | 250 | 1.60 | 81.83 | 12.82 | 11.06 | 16.64 | 211.93 |
| | | 14315 | | | | | | | |
| Ctrl | REGN10987 | 10987 | 150 | | NC | 199.09 | 278.92 | 176.15 | 236.08 |
| IgG | REGN14315 | 14315 | 150 | 0.22 | 1.03 | 6.78 | 12.70 | 9.38 | 254.95 |
| | REGN14315 + | 14315 + | 150 | 0.70 | 3.18 | 13.18 | 20.97 | 20.74 | 131.78 |
| | REGN10987 | 10987 | | | | | | | |

NC: Not calculated

### 8.1. Example 3: Neutralization Activity of REGN14287-Based Tetravalent 2×2 AFs

Since REGN14287 (comprising VH and VL domains from mAb14287 as set forth in Table 4) targets a different epitope from the previously tested 5 antibodies, a second set of 12 AF molecules (COVAF46-57) were generated by pairing REGN10933, 10987, 10985, 14315 and 14256 with REGN14287 anchor arm in both 2×2 N and C-Fab formats. Two 14287×14287 self-pairing AF molecules were also made for comparison (Tables 7 and 8). These AFs, corresponding parental IgGs, and the combination of REGN10933/10987 were tested in pseudovirus neutralization assay as described in Section 8.1.2, using D614G and various Omicron variants. Neutralization potency (IC50) and fold change of individual AF IC50 over REGN10933/10987 IC50 against D614G were determined.

Two tetravalent monospecific AFs, 2×2 N-Fab COVAF-51 and 2×2 C-Fab COVAF-57 (14287×14287) demonstrated the broadest and strongest potency against all Omicron variants (BA.1, BA.2, BA.2.75, BA.4/5 tested, with an IC50 range between 1.2-2.9 E-11 M, which is within 1-2-fold of the potency for REGN10933/10987 against D614G (Table 10). All other tetravalent bispecific AFs displayed much more reduced (by at least 4-fold) neutralization potency against at least one Omicron variant (Table 10).

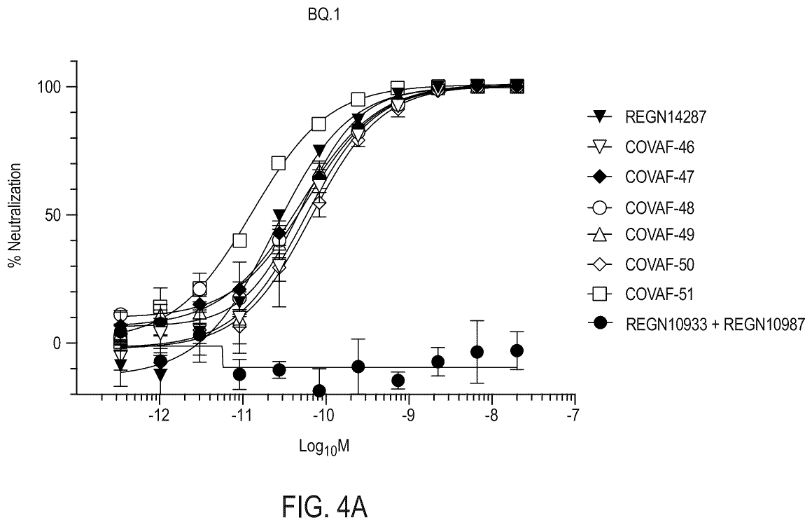

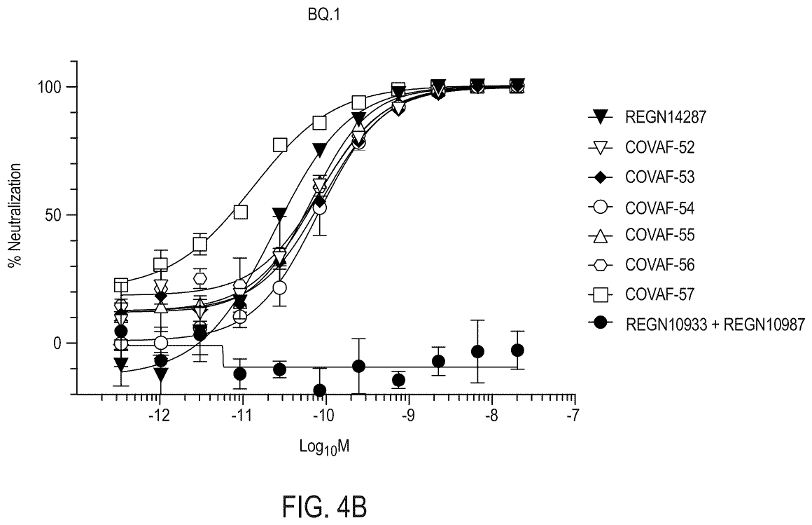

The neutralization activity of COVAF-41-57 against the currently circulating dominant Omicron variant BQ.1 was tested in the pseudovirus assay (FIG. 4A and 4B). Both COVAF-51 2×2 N-Fab and COVAF-57 2×2 C-Fab with monospecific tetravalent REGN14287 arms demonstrated superior activity to other bispecific bivalent AFs with IC50 at 1.3E-11 M (FIG. 4A and 4B). The potency for COVAF-51 and 57 is slightly better than parental REGN14287 IgG control and is similar to that of REGN10933/10987 against D614G variant (IC50 =1.2E-11 M). However, the neutralization for this IgG combination is totally lost against BQ. 1 (FIG. 4A and 4B).

TABLE 10
Summary of Pseudovirus Neutralization Activity of 2 × 2 N-Fab and C-Fab Pairing
REGN14287 With Self and Other REGN Arms Against D614G and Multiple Omicron Variants

| | | | | IC50 (M) | | | | |
|---|---|---|---|---|---|---|---|---|
| | | | MW | | | | | BA.4/ |
| Format | AF ID | REGN arms | (kDa) | D614G | BA.1 | BA.2 | BA.2.75 | BA.5 |
| 2 × 2 | COVAF-46 | 10933 × 14287 | 250 | 1.34E−11 | 2.71E−11 | 2.88E−11 | 2.28E−11 | 1.10E−10 |
| N-Fab | COVAF-47 | 14287 × 14256 | 250 | 2.23E−11 | 4.49E−11 | 3.24E−11 | 7.08E−11 | 1.01E−10 |
| | COVAF-48 | 10987 × 14287 | 250 | 1.71E−11 | 7.00E−11 | 8.24E−12 | 7.93E−11 | 2.38E−11 |
| | COVAF-49 | 14287 × 14315 | 250 | 5.15E−11 | 2.71E−11 | 2.37E−11 | 5.51E−11 | 6.40E−11 |
| | COVAF-50 | 10985 × 14287 | 250 | 1.72E−10 | 3.19E−11 | 5.17E−11 | 8.75E−11 | 1.15E−10 |
| | COVAF-51 | 14287 × 14287 | 250 | 5.57E−11 | 2.05E−11 | 1.23E−11 | 1.73E−11 | 2.94E−11 |
| 2 × 2 | COVAF-52 | 10933 × 14287 | 250 | 2.94E−11 | 3.39E−11 | 2.95E−11 | 3.72E−11 | 1.43E−10 |
| C-Fab | COVAF-53 | 14287 × 14256 | 250 | 3.18E−11 | 4.28E−11 | 5.38E−11 | 8.15E−11 | 1.80E−10 |
| | COVAF-54 | 10987 × 14287 | 250 | 2.39E−11 | 6.68E−11 | 2.17E−11 | 1.57E−10 | 3.80E−11 |
| | COVAF-55 | 14287 × 14315 | 250 | 3.36E−11 | 3.29E−11 | 4.48E−11 | 7.47E−11 | 9.37E−11 |
| | COVAF-56 | 10985 × 14287 | 250 | 1.08E−10 | 3.73E−11 | 8.12E−11 | 1.08E−10 | 1.38E−10 |
| | COVAF-57 | 14287 × 14287 | 250 | 3.42E−11 | 2.50E−11 | 1.44E−11 | 2.29E−11 | 2.46E−11 |
| Control | REGN14287 | 14287 | 150 | 9.06E−11* | 4.81E−11* | 2.84E−11* | 5.78E−11* | 5.00E−11* |
| IgG | REGN10933 | 10933 | 150 | 2.91E−11 | NC | NC | 2.17E−09 | NC |
| | REGN14256 | 14256 | 150 | 1.14E−11 | NC | NC | NC | NC |
| | REGN10987 | 10987 | 150 | 1.31E−11 | NC | 2.18E−09 | NC | 3.46E−09 |
| | REGN10933 + | 10933 + | 150 | 1.47E−11 | NC | 4.77E−09 | NC | 3.37E−09 |
| | REGN10987 | 10987 | | | | | | |
| | REGN14315 | 14315 | 150 | 8.78E−12 | 4.04E−11 | 1.68E−10 | 1.28E−09 | 1.59E−10 |
| | REGN14287 + | 14287 + | 150 | 1.11E−11 | 8.96E−11 | 1.43E−10 | 3.00E−10 | 2.19E−10 |
| | REGN14315 | 14315 | | | | | | |

| | | | | Fold Change over R10933 + R10987 against D614G | | | | |
|---|---|---|---|---|---|---|---|---|
| | | | MW | | | | | BA.4/ |
| Format | AF ID | REGN arms | (kDa) | D614G | BA.1 | BA.2 | BA.2.75 | BA.5 |
| 2 × 2 | COVAF-46 | 10933 × 14287 | 250 | 0.91 | 1.85 | 1.96 | 1.55 | 7.47 |
| N-Fab | COVAF-47 | 14287 × 14256 | 250 | 1.52 | 3.06 | 2.21 | 4.83 | 6.91 |
| | COVAF-48 | 10987 × 14287 | 250 | 1.16 | 4.77 | 0.56 | 5.41 | 1.63 |
| | COVAF-49 | 14287 × 14315 | 250 | 3.52 | 1.85 | 1.62 | 3.76 | 4.37 |
| | COVAF-50 | 10985 × 14287 | 250 | 11.73 | 2.18 | 3.53 | 5.97 | 7.85 |
| | COVAF-51 | 14287 × 14287 | 250 | 3.80 | 1.40 | 0.84 | 1.18 | 2.01 |
| 2 × 2 | COVAF-52 | 10933 × 14287 | 250 | 2.00 | 2.31 | 2.01 | 2.54 | 9.78 |
| C-Fab | COVAF-53 | 14287 × 14256 | 250 | 2.17 | 2.92 | 3.67 | 5.56 | 12.31 |
| | COVAF-54 | 10987 × 14287 | 250 | 1.63 | 4.56 | 1.48 | 10.72 | 2.59 |
| | COVAF-55 | 14287 × 14315 | 250 | 2.29 | 2.24 | 3.06 | 5.10 | 6.39 |
| | COVAF-56 | 10985 × 14287 | 250 | 7.37 | 2.55 | 5.54 | 7.34 | 9.39 |
| | COVAF-57 | 14287 × 14287 | 250 | 2.33 | 1.71 | 0.98 | 1.56 | 1.68 |
| Control | REGN14287 | 14287 | 150 | 6.18 | 3.28 | 1.94 | 3.94 | 3.41 |
| IgG | REGN10933 | 10933 | 150 | 1.98 | NC | NC | 148.02 | NC |
| | REGN14256 | 14256 | 150 | 0.77 | NC | NC | NC | NC |
| | REGN10987 | 10987 | 150 | 0.89 | NC | 148.77 | NC | 235.95 |
| | REGN10933 + | 10933 + | 150 | 1.00 | NC | 325.51 | NC | 229.67 |
| | REGN10987 | 10987 | | | | | | |
| | REGN14315 | 14315 | 150 | 0.60 | 2.76 | 11.47 | 87.04 | 10.83 |
| | REGN14287 + | 14287 + | 150 | 0.76 | 6.11 | 9.75 | 20.48 | 14.95 |
| | REGN14315 | 14315 | | | | | | |

*Mean value calculated based on two or three independent runs.
NC: Not calculated

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A multivalent anti-spike protein binding molecule comprising at least 4 anti-spike protein antigen-binding domains (ABDs) operably linked by one or more multimerization moieties.

2. The multivalent anti-spike protein binding molecule of claim 1, which is tetravalent.

3. The multivalent anti-spike protein binding molecule of claim 1, comprising:
(a) a first half antibody comprising:
(i) a first ABD comprising a first VH and a first VL;
(ii) a first linker;
(iii) a second ABD comprising a second VH and a second VL;
(iv) a first hinge domain; and
(v) a first Fc domain; and
(b) a second half antibody comprising:
(i) a third ABD comprising a third VH and a third VL;
(ii) a second linker;
(iii) a fourth ABD comprising a fourth VH and a fourth VL;
(iv) a second hinge domain; and
(v) a second Fc domain.

4. The multivalent anti-spike protein binding molecule of claim 1:
(a) a first half antibody comprising:
(i) a first ABD, typically comprising a first VH and a first VL;
(ii) a first hinge domain;
(iii) a first Fc domain;
(iv) a first linker; and
(v) a second ABD, typically comprising a second VH and a second VL; and
(b) a second half antibody comprising:
(i) a third ABD, typically comprising a third VH and a third VL;
(ii) a second hinge domain;
(iii) a second Fc domain;
(iv) a second linker; and
(v) a fourth ABD, typically comprising a fourth VH and a fourth VL.

5. The multivalent anti-spike protein binding molecule of claim 3, wherein the first Fc domain and the second Fc domain form an Fc homodimer.

6. The multivalent anti-spike protein binding molecule of claim 3, wherein the first Fc domain and the second Fc domain form an Fc heterodimer.

7. The multivalent anti-spike protein binding molecule of claim 3, wherein the first, second, third and fourth ABDs are the same.

8. The multivalent anti-spike protein binding molecule of claim 3, wherein the first and second ABDs are the same.

9. The multivalent anti-spike protein binding molecule of claim 8, wherein the third and fourth ABDs are the same.

10. The multivalent anti-spike protein binding molecule of claim 9, wherein the third and fourth ABDs are different from the first and second ABDs.

11. The multivalent anti-spike protein binding molecule of claim 3, wherein the first and third ABDs are the same.

12. The multivalent anti-spike protein binding molecule of claim 11, wherein the second and fourth ABDs are the same.

13. The multivalent anti-spike protein binding molecule of claim 12, wherein the second and fourth ABDs are different from the first and third ABDs.

14. The multivalent anti-spike protein binding molecule of claim 1, wherein the ABDs are human or humanized.

15. The multivalent anti-spike protein binding molecule of claim 1, wherein one or more (or all) ABDs are neutralizing.

16. The multivalent anti-spike protein binding molecule of claim 1, wherein the multimerization moieties are IgG Fc domains.

17. The multivalent anti-spike protein binding molecule of claim 16, wherein the IgG domains are IgG1 domains.

18. The multivalent anti-spike protein binding molecule of claim 16, wherein the IgG domains are IgG4 domains.

19.-22. (canceled)

23. The multivalent anti-spike protein binding molecule of claim 1, wherein one or more of the ABDs comprises:
(a) a VH comprising CDR-H1, CDR-H2, and CDR-H3 having the amino acid sequences of SEQ ID NOs: 579, 580, and 581, respectively; and
(b) a VL comprising CDR-L1, CDR-L2, and CDR-L3 having the amino acid sequences of SEQ ID NOs: 398, 372, and 583, respectively.

24. The multivalent anti-spike protein binding molecule of claim 1, in which at least two of the ABDs are Fab domains.

25. The multivalent anti-spike protein binding molecule of claim 1, in which all the ABDs are Fab domains.

26. The multivalent anti-spike protein binding molecule of claim 1, in which at least two of the ABDs are scFvs.

27. The multivalent anti-spike protein binding molecule of claim 1, in which at least all of the ABDs are scFvs.

28. The multivalent anti-spike protein binding molecule of claim 1, which is monospecific.

29. The multivalent anti-spike protein binding molecule of claim 1, which is multispecific.

30. The multivalent anti-spike protein binding molecule of claim 29, which is bispecific.

31. A nucleic acid or plurality of nucleic acids encoding the multivalent anti-spike protein binding molecule of claim 1.

32. A host cell engineered to express the multivalent anti-spike protein binding molecule of claim 1.

33. A method of producing a multivalent anti-spike protein binding molecule, comprising culturing the host cell of claim 32 and recovering the multivalent anti-spike protein binding molecule expressed thereby.

34. A pharmaceutical composition comprising the multivalent anti-spike protein binding molecule of claim 1 and an excipient.

35. A method of treating a coronavirus disease, comprising administering to a subject in need thereof the multivalent anti-spike protein binding molecule of claim 1.

36. A method of inhibiting an interaction between a RBD of a coronavirus and cellular ACE2, comprising administering to a subject in need thereof the multivalent anti-spike protein binding molecule of claim 1.

37.-47. (canceled)

48. The multivalent anti-spike protein binding molecule of claim 4, wherein the first Fc domain and the second Fc domain form an Fc homodimer.

49. The multivalent anti-spike protein binding molecule of claim 4, wherein the first Fc domain and the second Fc domain form an Fc heterodimer.

50. The multivalent anti-spike protein binding molecule of claim 4, wherein the first, second, third and fourth ABDs are the same.

51. The multivalent anti-spike protein binding molecule of claim 4, wherein the first and second ABDs are the same.

52. The multivalent anti-spike protein binding molecule of claim 51, wherein the third and fourth ABDs are the same.

53. The multivalent anti-spike protein binding molecule of claim 52, wherein the third and fourth ABDs are different from the first and second ABDs.

54. The multivalent anti-spike protein binding molecule of claim 4, wherein the first and third ABDs are the same.

55. The multivalent anti-spike protein binding molecule of claim 54, wherein the second and fourth ABDs are the same.

56. The multivalent anti-spike protein binding molecule of claim 55, wherein the second and fourth ABDs are different from the first and third ABDs.


SEQUENCE LISTING (752 sequences)

SEQ ID NO: 1 - protein, 120 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 2 - protein, 8 aa, synthetic construct
GFTFSDYY

SEQ ID NO: 3 - protein, 8 aa, synthetic construct
ITYSGSTI

SEQ ID NO: 4 - protein, 13 aa, synthetic construct
ARDRGTTMVPFDY

SEQ ID NO: 5 - protein, 107 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 6 - protein, 6 aa, synthetic construct
QDITNY

SEQ ID NO: 7 - protein, 9 aa, synthetic construct
QQYDNLPLT

SEQ ID NO: 8 - protein, 450 aa, synthetic construct
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 360 bp, synthetic construct
  source 1..360: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctca

SEQ ID NO: 11 - DNA, 24 bp, synthetic construct
  source 1..24: mol_type = other DNA
ggattcaccttcagtgactactac

SEQ ID NO: 12 - DNA, 24 bp, synthetic construct
  source 1..24: mol_type = other DNA
attacttatagtggtagtaccata

SEQ ID NO: 13 - DNA, 39 bp, synthetic construct
  source 1..39: mol_type = other DNA
gcgagagatcgcggtacaactatggtcccctttgactac

SEQ ID NO: 14 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaa

SEQ ID NO: 15 - DNA, 18 bp, synthetic construct
  source 1..18: mol_type = other DNA
caggacattaccaactat

SEQ ID NO: 16 - DNA, 27 bp, synthetic construct
  source 1..27: mol_type = other DNA
caacagtatgataatctccctctcact

SEQ ID NO: 17 - DNA, 1353 bp, synthetic construct
  source 1..1353: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 18 - protein, 235 aa, synthetic construct
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 645 bp, synthetic construct
  source 1..645: mol_type = other DNA
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 120 aa, synthetic construct
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSS

SEQ ID NO: 21 - protein, 8 aa, synthetic construct
GITFSNAW

SEQ ID NO: 22 - protein, 10 aa, synthetic construct
IKSKTDGGTT

SEQ ID NO: 23 - protein, 11 aa, synthetic construct
TTARWDWYFDL

SEQ ID NO: 24 - protein, 107 aa, synthetic construct
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIK

SEQ ID NO: 25 - protein, 6 aa, synthetic construct
QDIWNY

SEQ ID NO: 26 - protein, 9 aa, synthetic construct
QQHDDLPPT

SEQ ID NO: 27 - protein, 450 aa, synthetic construct
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, synthetic construct
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 360 bp, synthetic construct
  source 1..360: mol_type = other DNA
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctca

SEQ ID NO: 30 - DNA, 24 bp, synthetic construct
  source 1..24: mol_type = other DNA
ggaatcactttcagtaacgcctgg

SEQ ID NO: 31 - DNA, 30 bp, synthetic construct
  source 1..30: mol_type = other DNA
attaaaagcaaaactgatggtgggacaaca

SEQ ID NO: 32 - DNA, 33 bp, synthetic construct
  source 1..33: mol_type = other DNA
accacagcgaggtgggactggtacttcgatctc

SEQ ID NO: 33 - DNA, 321 bp, synthetic construct
  source 1..321: mol_type = other DNA
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaa

SEQ ID NO: 34 - DNA, 18 bp, synthetic construct
  source 1..18: mol_type = other DNA
caggacatttggaattat

SEQ ID NO: 35 - DNA, 27 bp, synthetic construct
  source 1..27: mol_type = other DNA
caacagcatgatgatctccctccgacc

SEQ ID NO: 36 - DNA, 1353 bp, synthetic construct
  source 1..1353: mol_type = other DNA
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 37 - DNA, 645 bp, synthetic construct
  source 1..645: mol_type = other DNA
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 38 - protein, 120 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 39 - protein, 8 aa, synthetic construct
GFTFSNYA

SEQ ID NO: 40 - protein, 8 aa, synthetic construct
ISYDGSNK

SEQ ID NO: 41 - protein, 13 aa, synthetic construct
ASGSDYGDYLLVY

SEQ ID NO: 42 - protein, 110 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 43 - protein, 9 aa, synthetic construct
SSDVGGYNY

SEQ ID NO: 44 - protein, 10 aa, synthetic construct
NSLTSISTWV

SEQ ID NO: 45 - protein, 450 aa, synthetic construct
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 216 aa, synthetic construct
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 47 - DNA, 360 bp, synthetic construct
  source 1..360: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctca

SEQ ID NO: 48 - DNA, 24 bp, synthetic construct
  source 1..24: mol_type = other DNA
ggattcaccttcagtaactatgct

SEQ ID NO: 49 - DNA, 24 bp, synthetic construct
  source 1..24: mol_type = other DNA
atatcatatgatggaagtaataaa

SEQ ID NO: 50 - DNA, 39 bp, synthetic construct
  source 1..39: mol_type = other DNA
gcgagtggctccgactacggtgactacttattggtttac

SEQ ID NO: 51 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtccta

SEQ ID NO: 52 - DNA, 27 bp, synthetic construct
  source 1..27: mol_type = other DNA
agcagtgacgttggtggttataactat

SEQ ID NO: 53 - DNA, 30 bp, synthetic construct
  source 1..30: mol_type = other DNA
aactctttgacaagcatcagcacttgggtg

SEQ ID NO: 54 - DNA, 1353 bp, synthetic construct
  source 1..1353: mol_type = other DNA
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 55 - DNA, 651 bp, synthetic construct
  source 1..651: mol_type = other DNA
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 56 - protein, 123 aa, synthetic construct
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSS

SEQ ID NO: 57 - protein, 8 aa, synthetic construct
GYIFTGYY

SEQ ID NO: 58 - protein, 8 aa, synthetic construct
INPNSGGA

SEQ ID NO: 59 - protein, 16 aa, synthetic construct
ARGSRYDWNQNNWFDP

SEQ ID NO: 60 - protein, 110 aa, synthetic construct
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVL

SEQ ID NO: 61 - protein, 9 aa, synthetic construct
SSDVGTYNY

SEQ ID NO: 62 - protein, 10 aa, synthetic construct
SSFTTSSTVV

SEQ ID NO: 63 - protein, 453 aa, synthetic construct
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 216 aa, synthetic construct
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 65 - DNA, 369 bp, synthetic construct
  source 1..369: mol_type = other DNA
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctca

SEQ ID NO: 66 - DNA, 24 bp, synthetic construct
  source 1..24: mol_type = other DNA
ggatacatcttcaccggctactat

SEQ ID NO: 67 - DNA, 24 bp, synthetic construct
  source 1..24: mol_type = other DNA
atcaaccctaacagtggtggcgca

SEQ ID NO: 68 - DNA, 48 bp, synthetic construct
  source 1..48: mol_type = other DNA
gcgagaggatcccggtatgactggaaccagaacaactggttcgacccc

SEQ ID NO: 69 - DNA, 330 bp, synthetic construct
  source 1..330: mol_type = other DNA
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtccta

SEQ ID NO: 70 - DNA, 27 bp, synthetic construct
  source 1..27: mol_type = other DNA
agcagtgacgttggtacttataactat

SEQ ID NO: 71 - DNA, 30 bp, synthetic construct
  source 1..30: mol_type = other DNA
agctcatttacaaccagcagcactgtggtt

SEQ ID NO: 72 - DNA, 1362 bp, synthetic construct
  source 1..1362: mol_type = other DNA
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 73 - DNA, 651 bp, synthetic construct
  source 1..651: mol_type = other DNA
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 74 - protein, 15 aa, synthetic construct
GGGGSGGGGSGGGGS

SEQ ID NO: 75 - protein, 231 aa, synthetic construct
DKRVESKYGPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 79 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 80 - protein, 5 aa, synthetic construct
GGGGS

SEQ ID NO: 81 - protein, 10 aa, synthetic construct
GGGGSGGGGS

SEQ ID NO: 82 - protein, 20 aa, synthetic construct
GGGGSGGGGSGGGGSGGGGS

SEQ ID NO: 83 - protein, 25 aa, synthetic construct
GGGGSGGGGSGGGGSGGGGSGGGGS

SEQ ID NO: 84 - protein, 30 aa, synthetic construct
GGGGSGGGGSGGGGSGGGGSGGGGSGGGGS

SEQ ID NO: 85 - protein, 4 aa, synthetic construct
CPPC

SEQ ID NO: 86 - protein, 4 aa, Homo sapiens
CPSC

SEQ ID NO: 87 - protein, 36 aa, synthetic construct
  VARIANT 1..5: note = This region may encompass 0-5 residues
  VARIANT 7..11: note = This region may encompass 0-5 residues
  VARIANT 13..17: note = This region may encompass 0-5 residues
  VARIANT 19..23: note = This region may encompass 0-5 residues
  VARIANT 25..29: note = This region may encompass 0-5 residues
  VARIANT 31..35: note = This region may encompass 0-5 residues
  VARIANT 1..36: note = This sequence may encompass 1-6 (GnS) repeating units, wherein n is 0-5
GGGGGSGGGGGSGGGGGSGGGGGSGGGGGSGGGGGS

SEQ ID NO: 88 - protein, 120 aa, synthetic construct
EVQLVESGGGLVQPGGSLRLSCAASGFTFRNYEMNWVRQAPGKGLEWVSYITSSGSTMYYADSVKGRFTISRDNAKNSLYLQMNSLRAEDTAVYYCARDGFYYYYAMDVWGQGTTVTVSS

SEQ ID NO: 89 - protein, 8 aa, synthetic construct
GFTFRNYE

SEQ ID NO: 90 - protein, 8 aa, synthetic construct
ITSSGSTM

SEQ ID NO: 91 - protein, 13 aa, synthetic construct
ARDGFYYYYAMDV

SEQ ID NO: 92 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVGRVFGTGTKVTVL

SEQ ID NO: 93 - protein, 8 aa, synthetic construct
SSNIGNNY

SEQ ID NO: 94 - skipped

SEQ ID NO: 95 - protein, 12 aa, synthetic construct
GTWDSSLSVGRV

SEQ ID NO: 96 - protein, 116 aa, synthetic construct
EVQLVESGGGLVQPGGSLRLSCAASGFTVSSNYMNWVRQAPGKGLEWVSVIYSGGSTFYADSVKGRFTISRHNSKNTLYLQMNSLRPEDTAVYYCARDLGGYFDYWGQGTLVTVSS

SEQ ID NO: 97 - protein, 8 aa, synthetic construct
GFTVSSNY

SEQ ID NO: 98 - protein, 7 aa, synthetic construct
IYSGGST

SEQ ID NO: 99 - protein, 10 aa, synthetic construct
ARDLGGYFDY

SEQ ID NO: 100 - protein, 106 aa, synthetic construct
DIQMTQSPSTLSASVGDRVTITCRASQSFSSWLAWYQQKPGKAPKLLIYKASSLESGVPSRFSGSGSGTEFTLTISSLQPDDFATYYCQQYNSYYTFGQGTKLEIK

SEQ ID NO: 101 - protein, 6 aa, synthetic construct
QSFSSW

SEQ ID NO: 102 - skipped

SEQ ID NO: 103 - protein, 8 aa, synthetic construct
QQYNSYYT

SEQ ID NO: 104 - protein, 117 aa, synthetic construct
EVQLVESGGGLVQPGGSLRLSCAASGLIVSRNYMIWVRQAPGKGLEWVSVIYSGGSTFYADSVKGRFTISRHNSKNTLYLQMNSLRAEDTAVYYCARDLGTGGMDVWGQGTTVTVSS

SEQ ID NO: 105 - protein, 8 aa, synthetic construct
GLIVSRNY

SEQ ID NO: 106 - protein, 11 aa, synthetic construct
ARDLGTGGMDV

SEQ ID NO: 107 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 108 - protein, 6 aa, synthetic construct
QGISSY

SEQ ID NO: 109 - skipped

SEQ ID NO: 110 - protein, 9 aa, synthetic construct
QQLNSYPLT

SEQ ID NO: 111 - protein, 120 aa, synthetic construct
QVQLVESGGGVVQPGRSLRLSCAASGFTFSYYGMHWVRQAPGKGLEWVAVIWYDGSNKFYADSVKGRFTISRDNSKNTLYLQMNSLRAEDTAVYYCAAARPGYYYGMDVWGQGTTVTVSS

SEQ ID NO: 112 - protein, 8 aa, synthetic construct
GFTFSYYG

SEQ ID NO: 113 - protein, 8 aa, synthetic construct
IWYDGSNK

SEQ ID NO: 114 - protein, 13 aa, synthetic construct
AAARPGYYYGMDV

SEQ ID NO: 115 - protein, 107 aa, synthetic construct
DIQMTQSPSSLSASVGDRVTITCRASQGISNYLAWFQQKPGKAPNSLIYAASSLQRGVPSKFSGSGSGTDFTLTISSLQPEDFATYYCQQYNSYPRTFGQGTKVEIK

SEQ ID NO: 116 - protein, 6 aa, synthetic construct
QGISNY

SEQ ID NO: 117 - protein, 9 aa, synthetic construct
QQYNSYPRT

SEQ ID NO: 118 - protein, 117 aa, synthetic construct
EVQLVESGGGLVQPGGSLRLSCAASGITVSSNYMSWVRQAPGKGLEWVSVIYSGGSTFYADSVKGRFTISRHNSKNTLYLQMNSLRAEDTAVYYCARERNFDAFDIWGQGTMVTVSS

SEQ ID NO: 119 - protein, 8 aa, synthetic construct
GITVSSNY

SEQ ID NO: 120 - protein, 11 aa, synthetic construct
ARERNFDAFDI

SEQ ID NO: 121 - protein, 108 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLFTFGPGTKVDIK

SEQ ID NO: 122 - protein, 6 aa, synthetic construct
QGISSW

SEQ ID NO: 123 - protein, 10 aa, synthetic construct
QQANSFPLFT

SEQ ID NO: 124 - protein, 119 aa, synthetic construct
EVQLVESGGGLVQPGGSLRLSCAASGFTFSIYEMNWVRQAPGKGLEWVSYITSSGTTIYYADSVKGRFTISRDNAKNSLYLQMNSLRAEDTAVYYCASSSSSGYYFDYWGQGTLVTVSS

SEQ ID NO: 125 - protein, 8 aa, synthetic construct
GFTFSIYE

SEQ ID NO: 126 - protein, 8 aa, synthetic construct
ITSSGTTI

SEQ ID NO: 127 - protein, 12 aa, synthetic construct
ASSSSSGYYFDY

SEQ ID NO: 128 - protein, 113 aa, synthetic construct
DIVMTQSPDSLAVSLGERATINCKSSQSVLYSSNNKNYLAWYQQKPGQPPNLLIYWASTRESGVPDRFSGSGSGTDFTLTISSLQAEDVAVYYCQQYYSTPPTFGQGTKLEIK

SEQ ID NO: 129 - protein, 12 aa, synthetic construct
QSVLYSSNNKNY

SEQ ID NO: 130 - skipped

SEQ ID NO: 131 - protein, 9 aa, synthetic construct
QQYYSTPPT

SEQ ID NO: 132 - protein, 121 aa, synthetic construct
QVQLVQSGAEVKKPGASVKVSCKASGYTFINYYIHWVRQAPGQGLEWMGWINPNSGGTNYAQKFQGRVTMTRDTSISTAYMELSRLRSDDTAVYYCAIITIFGVVTWFDPWGQGTLVTVSS

SEQ ID NO: 133 - protein, 8 aa, synthetic construct
GYTFINYY

SEQ ID NO: 134 - protein, 8 aa, synthetic construct
INPNSGGT

SEQ ID NO: 135 - protein, 14 aa, synthetic construct
AIITIFGVVTWFDP

SEQ ID NO: 136 - protein, 107 aa, synthetic construct [sequence omitted]

SEQ ID NO: 137 - protein, 6 aa, synthetic construct
QSISSY

SEQ ID NO: 138 - protein, 9 aa, synthetic construct
QQSYSTPLT

SEQ ID NO: 139 - protein, 115 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARDPLLIDYWGQGTLVTVSS

SEQ ID NO: 140 - protein, 8 aa, synthetic construct
GGSISSYY

SEQ ID NO: 141 - protein, 7 aa, synthetic construct
IYYSGST

SEQ ID NO: 142 - protein, 9 aa, synthetic construct
ARDPLLIDY

SEQ ID NO: 143 - protein, 108 aa, synthetic construct
EIVLTQSPGTLSLSPGERATLSCRASQSVSRSYLAWYQQKPGQAPRLLIYGVSSRATGIPDRFSGSGSGTDFTLTISRLEPEDFAVYYCQQYGSSPQTFGQGTKLEIK

SEQ ID NO: 144 - protein, 7 aa, synthetic construct
QSVSRSY

SEQ ID NO: 145 - skipped

SEQ ID NO: 146 - protein, 9 aa, synthetic construct
QQYGSSPQT

SEQ ID NO: 147 - protein, 125 aa, synthetic construct
EVQLVESGGGLVQPGRSLRLSCAASGFTFDDYAMYWVRQAPGKGLEWVSGISWNSGSIGYADSVKGRFTISRDNAKNSLYLQMNSLRAADTALYYCAKDKDWNSRGYYYYGMDVWGQGTTVTVSS

SEQ ID NO: 148 - protein, 8 aa, synthetic construct
GFTFDDYA

SEQ ID NO: 149 - protein, 8 aa, synthetic construct
ISWNSGSI

SEQ ID NO: 150 - protein, 18 aa, synthetic construct
AKDKDWNSRGYYYYGMDV

SEQ ID NO: 151 - protein, 107 aa, synthetic construct
DVVMTQSPSSLSASVGDRVTITCRASQSISSYLNWYQQKPGKAPKLLIYAASSLQSGVPSRFSGSGSGTDFTLTISSLQPEDFATYYCQQSYITPYTFGQGTKVEIK

SEQ ID NO: 152 - protein, 9 aa, synthetic construct
QQSYITPYT

SEQ ID NO: 153 - protein, 116 aa, synthetic construct
EVQLVESGGGLVQSGGSLRLSCAASGLTVSSNYMSWVRQAPGKGLEWVSVIYSGGSTFYSDSVKGRFTISRHNSKNTLYLQMNSLRAEDTAVYYCARARGDAFDIWGQGTMVTVSS

SEQ ID NO: 154 - protein, 8 aa, synthetic construct
GLTVSSNY

SEQ ID NO: 155 - protein, 10 aa, synthetic construct
ARARGDAFDI

SEQ ID NO: 156 - protein, 108 aa, synthetic construct
DIQMTQSPSSLSASVGDRVTITCQASQGISNYLNWYQQKPGKAPKLLISDASNLETGVPSRFSGSGSGTDFTFTISSLQPEDIATYYCQQYDNLRFFTFGPGTKVDIK

SEQ ID NO: 157 - skipped

SEQ ID NO: 158 - protein, 10 aa, synthetic construct
QQYDNLRFFT

SEQ ID NO: 159-172 - skipped (14 entries)

SEQ ID NO: 173 - protein, 120 aa, synthetic construct
EVQLVESGGGLVQPGGSLRLSCAASGFTVSSNYMSWVRQAPGKGLEWVSLIYSGGSTYYADSVKGRFTISRHNSKNTLYLQMNSLRSEDTAVYYCARVPLGDYYYGMDVWGQGTTVTVSS

SEQ ID NO: 174 - protein, 14 aa, synthetic construct
ARVPLGDYYYGMDV

SEQ ID NO: 175 - protein, 107 aa, synthetic construct
DIQMTQSPSSLSASVGDRVTITCQASQDINKYLNWYQQKPGKAPKLLIYDASNLETGVPSRFSGSGSGTDFTFTISSLQPEDIATYYCQQSDNLPLTFGGGTKVEIK

SEQ ID NO: 176 - protein, 6 aa, synthetic construct
QDINKY

SEQ ID NO: 177 - protein, 9 aa, synthetic construct
QQSDNLPLT

SEQ ID NO: 178 - protein, 124 aa, synthetic construct
QVQLVESGGGVVQPGRSLRLSCAASGFTFRNYGMHWVRQAPGKGLEWVSVIWYDGSNRYYADSVKGRFTISRDTSKNTLYLQMNSLRAEDTAVYYCARDPPITGTTGGDAFDIWGQGTMVTVSS

SEQ ID NO: 179 - protein, 8 aa, synthetic construct
GFTFRNYG

SEQ ID NO: 180 - protein, 8 aa, synthetic construct
IWYDGSNR

SEQ ID NO: 181 - protein, 17 aa, synthetic construct
ARDPPITGTTGGDAFDI

SEQ ID NO: 182 - protein, 107 aa, synthetic construct
DIQMTQSPSSLSASVGDRVTITCRASQSISSYLNWYQQKPGKAPHLLIYAASSLQSGVPSRFSGSGSGTDFTLTISSLQPEDFATYYCQQSYSTPYTFGQGTKLEIK

SEQ ID NO: 183 - protein, 9 aa, synthetic construct
QQSYSTPYT

SEQ ID NO: 184 - protein, 119 aa, synthetic construct
QITLKESGPTLVKPTQTLTLTCTFSGFSLSTNVVGVGWIRQPPGKALEWLALIYWNDDKRYSPSLKSRLTITKDTSKNQVVLTMTNMDPVDTATYYCAHGPTGDYFDYWGQGTLVTVSS

SEQ ID NO: 185 - protein, 10 aa, synthetic construct
GFSLSTNVVG

SEQ ID NO: 186 - protein, 7 aa, synthetic construct
IYWNDDK

SEQ ID NO: 187 - protein, 11 aa, synthetic construct
AHGPTGDYFDY

SEQ ID NO: 188 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTLMYTFGQGTKLEIK

SEQ ID NO: 189 - protein, 10 aa, synthetic construct
QQTYSTLMYT

SEQ ID NO: 190 - protein, 122 aa, synthetic construct
EVQLVESGGGLVQPGRSLRLSCAASGFTFDDYAMHWVRQAPGKGLEWVSGVSWNSGTIDYADSVKGRFTIIRDNAKNSLYLQMNSLRAEDTALYYCSKDMGRLDYYSGLDVWGQGTTVTVSS

SEQ ID NO: 191 - protein, 8 aa, synthetic construct
VSWNSGTI

SEQ ID NO: 192 - protein, 15 aa, synthetic construct
SKDMGRLDYYSGLDV

SEQ ID NO: 193 - protein, 108 aa, synthetic construct
EIVLTQSPGTLSLSPGERATLSCRASQSVSSSDFAWYQQKPGQAPRLLIYGASSRATGIPDRFSGSGSGTDFTLTISRLEPEDFAVYYCQQYGSSPWTFGQGTKVEIK

SEQ ID NO: 194 - protein, 7 aa, synthetic construct
QSVSSSD

SEQ ID NO: 195 - skipped

SEQ ID NO: 196 - protein, 9 aa, synthetic construct
QQYGSSPWT

SEQ ID NO: 197 - protein, 118 aa, synthetic construct
EVQLVESGGGLVQPGGSLRLSCAVSGFTVSSNYMSWVRQAPGKGLEWVSLIYSGGSTFYADSVKGRFTISRHNSKNTLYLQMNSLRAEDTAVYYCAREGGYSYDYNYWGQGTLVTVSS

SEQ ID NO: 198 - protein, 12 aa, synthetic construct
AREGGYSYDYNY

SEQ ID NO: 199 - protein, 107 aa, synthetic construct
DIQMTQSPSSLSASVGDRVTITCQASQDIRNYLNWYQQKPGKAPKLLIYDASNLETGVPSRFSGIGSGTDFTFTISSLQPEDIATYYCQQYDNLPITFGQGTRLEIK

SEQ ID NO: 200 - protein, 6 aa, synthetic construct
QDIRNY

SEQ ID NO: 201 - protein, 9 aa, synthetic construct
QQYDNLPIT

SEQ ID NO: 202 - protein, 117 aa, synthetic construct
EVQLVESGGGLVQPGGSLRLSCAASGLTVSSNYMNWVRQVPGKGLEWVSVIYSGGSKFYVDSVKGRFTISRHNSKNTLYLQMNSLRPEDTAVYYCTRDAQYYGMDVWGQGTTVTVSS

SEQ ID NO: 203 - protein, 7 aa, synthetic construct
IYSGGSK

SEQ ID NO: 204 - protein, 11 aa, synthetic construct
TRDAQYYGMDV

SEQ ID NO: 205 - protein, 108 aa, synthetic construct
DIQMTQSPSSLSASVGDRVTITCRASQSISTYLHWYQQKPGEAPKLLIYAASSLQSGVPSRFSGSGSGTDFTLTISSLQPEDFATYYCQQSYSTPPEIFGQGTKLEIK

SEQ ID NO: 206 - protein, 6 aa, synthetic construct
QSISTY

SEQ ID NO: 207 - protein, 10 aa, synthetic construct
QQSYSTPPEI

SEQ ID NO: 208 - protein, 118 aa, synthetic construct
EVQLVESGGGLVQPGGSLRLSCAASGITVSSNYMTWVRQAPGKGLEWVSVIYSGGSTFYADSVKGRFTISRHNSKNTLYLQMNNLRAEDTAVYYCVRDLEVRGGMDVWGQGTTVTVSS

SEQ ID NO: 209 - protein, 12 aa, synthetic construct
VRDLEVRGGMDV

SEQ ID NO: 210 - protein, 108 aa, synthetic construct
EIVLTQSPGTLSLSPGERATLSCRASQSFSSSYLAWYQQKPGQAPRLLIYGASSRATGIPDRFSGSGSGTDFTLTISRLEPEDFAVYYCQQYGSSPWTFGQGTKVEIK

SEQ ID NO: 211 - protein, 7 aa, synthetic construct
QSFSSSY

SEQ ID NO: 212 - protein, 119 aa, synthetic construct
EVQLVESGGGLVQPGGSLRLSCAASGITVSSNYMSWVRQAPGKGLEWVSLIYSGGSTYYADSVKGRFTISRDNSKNTLYLQMNSLRVEDTAVYYCARDHGMAAAGYNYWGQGTLVTVSS

SEQ ID NO: 213 - protein, 13 aa, synthetic construct
ARDHGMAAAGYNY

SEQ ID NO: 214 - protein, 107 aa, synthetic construct
DIQMTQSPSSLSASVGDRVTITCQASQDINKYLNWYQQKPGKAPKLLIYDASNLETGVPSRFSGSGSGTDFTFTINSLQPEDFATYFCQQYDNLPPAFGQGTKVEIK

SEQ ID NO: 215 - protein, 9 aa, synthetic construct
QQYDNLPPA

SEQ ID NO: 216 - protein, 115 aa, synthetic construct
EVQLVESGGGLVQPGGSLRVSCAASGFTVSRNYMSWVRQAPGKGLEWVSVIYSGGSTFYADSVKGRFTISRDNSKNTLYLQMNSLRAEDTAIYYCARYIPRFDPWGQGTLVTVSS

SEQ ID NO: 217 - protein, 8 aa, synthetic construct
GFTVSRNY

SEQ ID NO: 218 - protein, 9 aa, synthetic construct
ARYIPRFDP

SEQ ID NO: 219 - protein, 107 aa, synthetic construct
DIQMTQSPSSLSASVGDRVTITCQASQDISNYLNWFQQKPGRAPKLLIYDASNLETGVPSRFSGSGSGTDFTFTISSLQPEDIATYYCQQYDNLPITFGQGTRLEIK

SEQ ID NO: 220 - protein, 6 aa, synthetic construct
QDISNY

SEQ ID NO: 221 - protein, 117 aa, synthetic construct
EVQLVESGGGLVQPGGSLRLSCAASGLTVSSNYMSWVRQAPGKGLEWVSVIYSGGSTFYADSVKGRFTISRDNSKNTLYLQMNSLRAEDTAVYYCARDLIKYGMDVWGQGTTVTVSS

SEQ ID NO: 222 - protein, 11 aa, synthetic construct
ARDLIKYGMDV

SEQ ID NO: 223 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPHFGPGTKVDIK

SEQ ID NO: 224 - protein, 9 aa, synthetic construct
QQYDNLPPH

SEQ ID NO: 225 - protein, 117 aa, synthetic construct
EVQLVESGGGLVQPGGSLRLSCAASGVTVSSNYMSWVRQAPGKGLEWVSVIYSGGSTFYADSVKGRFTISRDNSKNTLFLQMNSLRIEDTAVYYCARDLGPYGMDVWGQGTTVTVSS

SEQ ID NO: 226 - protein, 8 aa, synthetic construct
GVTVSSNY

SEQ ID NO: 227 - protein, 11 aa, synthetic construct
ARDLGPYGMDV

SEQ ID NO: 228 - protein, 108 aa, synthetic construct
DIQLTQSPSFLSASVGDRVTITCRASQGISSYLAWYQQKPGKAPKLLIYAASTLQSGVPSRFSGSGSGTQFILTISSLQPEDFATYYCQQLNSYPGFTFGPGTKVDIK

SEQ ID NO: 229 - protein, 10 aa, synthetic construct
QQLNSYPGFT

SEQ ID NO: 230 - protein, 118 aa, synthetic construct
QVQLQESGPGLVKPSETLSLTCTVSGGSINNYYWIWIRQPPGKGLEWIGYIYHSGSTNYNPSLKSRVTISVDTSKNQFSLKVSSVTAADTAVYYCANMVRGVYEDDYWGQGTLVTVSS

SEQ ID NO: 231 - protein, 8 aa, synthetic construct
GGSINNYY

SEQ ID NO: 232 - protein, 7 aa, synthetic construct
IYHSGST

SEQ ID NO: 233 - protein, 12 aa, synthetic construct
ANMVRGVYEDDY

SEQ ID NO: 234 - protein, 107 aa, synthetic construct
DIQMTQSPSSLSASVGDRVTITCRASQSISNYLNWYQQKPGKAPKLLIFAASSLRSGVPSRFSGSGSGTDFTLTISSLHPEDFATYYCQQNYNTPLTFGPGTKVDIK

SEQ ID NO: 235 - protein, 6 aa, synthetic construct
QSISNY

SEQ ID NO: 236 - protein, 9 aa, synthetic construct
QQNYNTPLT

SEQ ID NO: 237 - protein, 122 aa, synthetic construct
EVQLVESGGGLIQPGGSLRLSCAASVFTVSYNYMSWVRQAPGKGLEWVSVIYSGGSTYYADSVKGRFTISRDNSKNTLYLQMNSLRAEDTAVYYCARALPGWGGSFEYFDYWGQGTLVTVSS

SEQ ID NO: 238 - protein, 8 aa, synthetic construct
VFTVSYNY

SEQ ID NO: 239 - protein, 16 aa, synthetic construct
ARALPGWGGSFEYFDY

SEQ ID NO: 240 - protein, 107 aa, synthetic construct
DIQMTQSPSTLSASVGDRVTITCRASQSISSWLAWYQQKPGKAPKLLIYKASSLESGVSSRFSGSGSGTEFTLTISSLQPDDFATYYCQQYNSYSVTFGQGTKVEIK

SEQ ID NO: 241 - protein, 6 aa, synthetic construct
QSISSW

SEQ ID NO: 242 - protein, 9 aa, synthetic construct
QQYNSYSVT

SEQ ID NO: 243 - protein, 121 aa, synthetic construct
EVQLVESGGGLVQPGGSLRLSCAASGLTVSSNYMSWVRQAPGKGLEWVSVIYSGGSTFYADSVKGRFTISRDNSKNTLYLQMNSLRPEDTAVYYCAREGRIAATGYGMDVWGQGTTVTVSS

SEQ ID NO: 244 - protein, 15 aa, synthetic construct
AREGRIAATGYGMDV

SEQ ID NO: 245 - protein, 107 aa, synthetic construct
DIQMTQSPSSLSASVGDRVTITCQASQDISNYLNWYQQKPGKAPKLLIYDASNLGTGVPSRFSGSRSGTDFTFTISSLQPEDIATYYCHQYDNLPQTFGPGTKVDIK

SEQ ID NO: 246 - protein, 9 aa, synthetic construct
HQYDNLPQT

SEQ ID NO: 247 - protein, 114 aa, synthetic construct
EVQLVESGGGLVQPGGSLRLSCAASGFTVSSNYMSWVRQAPGKGLEWVSVIYPGGSTFYADSVKGRFTISRDNSKNTLYLQMNSLRAEDTAVYYCARDQRDFAWGQGTLVTVSS

SEQ ID NO: 248 - protein, 7 aa, synthetic construct
IYPGGST

SEQ ID NO: 249 - protein, 8 aa, synthetic construct
ARDQRDFA

SEQ ID NO: 250 - protein, 107 aa, synthetic construct
DIQMTQSPSSLSASVGDRVTITCQASQDISNYLNWYQQKPGKAPKLLIYDASNLETGVPSRFSGSGSGTDFTFTISGLQPEDIATYYCQQYDNLPPTFGQGTRLEIK

SEQ ID NO: 251 - protein, 9 aa, synthetic construct
QQYDNLPPT

SEQ ID NO: 252 - protein, 127 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDGRTITMVRGVIDDAFDIWGQGTMVTVSS

SEQ ID NO: 253 - protein, 8 aa, synthetic construct
GFTFSSYA

SEQ ID NO: 254 - protein, 8 aa, synthetic construct
ISYDGSNK

SEQ ID NO: 255 - protein, 20 aa, synthetic construct
ARDGRTITMVRGVIDDAFDI

SEQ ID NO: 256 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGQGTRLEIK

SEQ ID NO: 257 - protein, 120 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNRLRADDTAVYYCVRDPVGRYYYGMDVWGQGTTVTVSS

SEQ ID NO: 258 - protein, 14 aa, synthetic construct
VRDPVGRYYYGMDV

SEQ ID NO: 259 - protein, 107 aa, synthetic construct
DIQMTQSPSSLSASVGDRVTITCQASQDINNYLNWYQQKPGKAPKLLIYDASNLETGVPSRFSGSGSGTDFTFTISSLQPEDIATYYCQQYDNLPRTFGGGTKVEIK

SEQ ID NO: 260 - protein, 6 aa, synthetic construct
QDINNY

SEQ ID NO: 261 - protein, 9 aa, synthetic construct
QQYDNLPRT

SEQ ID NO: 262 - protein, 117 aa, synthetic construct
EVQLVESGGGLIQPGGSLRLSCAASGVTVSSNYMSWVRQAPGKGLEWVSVIYSGGSTFYSDSVKGRFSISRDNSKNTLYLQMNSLRAEDTAVYYCARDLYYYGMDVWGQGTTVTVSS

SEQ ID NO: 263 - protein, 11 aa, synthetic construct
ARDLYYYGMDV

SEQ ID NO: 264 - protein, 108 aa, synthetic construct
DIQLTQSPSFLSASVGDRVTITCRASQGISTYLAWYQQKPGKAPKLLIYAASTLQSGVPSRFSGSGSGTEFTLTISSLQPEDFATYYCQQLDSYPGLTFGGGTKVEIK

SEQ ID NO: 265 - protein, 6 aa, synthetic construct
QGISTY

SEQ ID NO: 266 - protein, 10 aa, synthetic construct
QQLDSYPGLT

SEQ ID NO: 267 - protein, 123 aa, synthetic construct
QVQLVQSGAEVKKPGSSVKVSCKASGGTFSSYAITWVRQAPGQGLEWMGRIIPMFGIANYAQKFQGRVTITADKSTSTAYMEVSSLRSEDTAVYYCARTPFYYDSSGYYLDYWGQGTLVTVSS

SEQ ID NO: 268 - protein, 8 aa, synthetic construct
GGTFSSYA

SEQ ID NO: 269 - protein, 8 aa, synthetic construct
IIPMFGIA

SEQ ID NO: 270 - protein, 16 aa, synthetic construct
ARTPFYYDSSGYYLDY

SEQ ID NO: 271 - protein, 108 aa, synthetic construct
EIVLTQSPGTLSLSPGERATLSCRASQSVSSNYLAWYQQSPGQAPRLLIYGASSRATGIPDRFSGSGSGTDFTLTISRLEPEDFAVYYCQQYVRSPRTFGQGTKVEIK

SEQ ID NO: 272 - protein, 7 aa, synthetic construct
QSVSSNY

SEQ ID NO: 273 - protein, 9 aa, synthetic construct
QQYVRSPRT

SEQ ID NO: 274 - protein, 119 aa, synthetic construct
EVQLVESGGGLVQPGGSLRLSCAASGLTVSSNYMSWVRQAPGKGLEWVSVIYSGGTTYYADSVKGRFTISRHNSKNTLSLQMNSLRAEDTAVYYCARGEGANYYGMDVWGQGTTVTVSS

SEQ ID NO: 275 - protein, 7 aa, synthetic construct
IYSGGTT

SEQ ID NO: 276 - protein, 13 aa, synthetic construct
ARGEGANYYGMDV

SEQ ID NO: 277 - protein, 112 aa, synthetic construct
QSVLTQPPSVSGAPGQRVTISCTGSSSNIGTGYDVHWYQQLPGTAPKLLIYGNSNRPSGVPDRFSGSKSGTSASLAITGLQAEDEADYYCQSYDSSLSGYVVFGGGTKLTVL

SEQ ID NO: 278 - protein, 9 aa, synthetic construct
SSNIGTGYD

SEQ ID NO: 279 - skipped

SEQ ID NO: 280 - protein, 12 aa, synthetic construct
QSYDSSLSGYVV

SEQ ID NO: 281 - protein, 119 aa, synthetic construct
EVQLVESGGGLVQPGGSLRLSCAASGLTVNRNYMSWVRQAPGKGLEWVSLIYSGGSTYYADSVKGRFTISRDNSKNTLYLQMNSLRLEDTAVYYCARGELGIPYGMDVWGQGTTVTVSS

SEQ ID NO: 282 - protein, 8 aa, synthetic construct
GLTVNRNY

SEQ ID NO: 283 - protein, 13 aa, synthetic construct
ARGELGIPYGMDV

SEQ ID NO: 284 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGGGTKLTVL

SEQ ID NO: 285 - protein, 11 aa, synthetic construct
GTWDSSLSAGV

SEQ ID NO: 286 - protein, 128 aa, synthetic construct
EVQLVESGGGLVQPGRSLRLSCAASGFTFDDYAMHWVRQAPGKGLEWVSGISWNRGSIGYADSVKGRFTISRDNAKNSLYLQMSSLRAEDTALYYCAKDGERWDSVVVPSARNGMDVWGQGTTVTVSS

SEQ ID NO: 287 - protein, 8 aa, synthetic construct
ISWNRGSI

SEQ ID NO: 288 - protein, 21 aa, synthetic construct
AKDGERWDSVVVPSARNGMDV

SEQ ID NO: 289 - protein, 112 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 290 - protein, 9 aa, synthetic construct
SSNIGAGYD

SEQ ID NO: 291 - protein, 12 aa, synthetic construct
QSYDSSLSGSYV

SEQ ID NO: 292 - protein, 119 aa, synthetic construct
EVQLVESGGGLVQPGGSLRLSCAASEFTVSSNYMSWVRQAPGKGLEWVSVIYSGGSTFYADSVKGRFTISRDNSKNTLYLQMNSLRAEDTAVYYCARALPYGDLHFDYWGQGTLVTVSS

SEQ ID NO: 293 - protein, 8 aa, synthetic construct
EFTVSSNY

SEQ ID NO: 294 - protein, 13 aa, synthetic construct
ARALPYGDLHFDY

SEQ ID NO: 295 - protein, 112 aa, synthetic construct
QSVLTQPPSVSGAPGQRVTISCTGSSSNIGTGYDVHWYQQLPGTAPKLLIYGNTNRPSGVPDRFSGSKSGTSASLAITGLQAEDEADYYCQSYDSSLSDSYVFGTGTKVTVL

SEQ ID NO: 296 - skipped

SEQ ID NO: 297 - protein, 12 aa, synthetic construct
QSYDSSLSDSYV

SEQ ID NO: 298-304 - skipped (7 entries)

SEQ ID NO: 305 - protein, 127 aa, synthetic construct
EVQLVESGGGLVQPGGSLRLSCTASGFTFSNYEMNWVRQAPGKGLEWVSYISSRSGSTLHYADSVKGRFTISRDNAKNSLYLQMNSLRAEDTAVYYCARRGDGTSSLIHHYYYMDVWGKGTTVTVSS

SEQ ID NO: 306 - protein, 8 aa, synthetic construct
GFTFSNYE

SEQ ID NO: 307 - protein, 9 aa, synthetic construct
ISSRSGSTL

SEQ ID NO: 308 - protein, 19 aa, synthetic construct
ARRGDGTSSLIHHYYYMDV

SEQ ID NO: 309 - protein, 110 aa, synthetic construct
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGPVFGGGTKLTVL

SEQ ID NO: 310 - protein, 8 aa, synthetic construct
SSNIGSNT

SEQ ID NO: 311 - skipped

SEQ ID NO: 312 - protein, 11 aa, synthetic construct
AAWDDSLNGPV

SEQ ID NO: 313 - protein, 123 aa, synthetic construct; identical to SEQ ID NO: 56

SEQ ID NO: 314 - protein, 8 aa, synthetic construct
GYIFTGYY

SEQ ID NO: 315 - protein, 8 aa, synthetic construct
INPNSGGA

SEQ ID NO: 316 - protein, 16 aa, synthetic construct
ARGSRYDWNQNNWFDP

SEQ ID NO: 317 - protein, 110 aa, synthetic construct; identical to SEQ ID NO: 60

SEQ ID NO: 318 - protein, 9 aa, synthetic construct
SSDVGTYNY

SEQ ID NO: 319 - protein, 10 aa, synthetic construct
SSFTTSSTVV

SEQ ID NO: 320 - protein, 117 aa, synthetic construct
EVQMVESGGGLVQPGGSLRLSCAASGFTVSSNYMTWVRQAPGKGLEWVSVIYSGGSTYYADSVKGRFTISRHNSKNTLYLQMNSLRAEDTAVYYCARDGVSYGMDVWGQGTTVTVSS

SEQ ID NO: 321 - protein, 11 aa, synthetic construct
ARDGVSYGMDV

SEQ ID NO: 322 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 323 - protein, 10 aa, synthetic construct
QQSYSTPPIT

SEQ ID NO: 324 - protein, 119 aa, synthetic construct
EVQLVESGGGLVHPGGSLRLSCAASGLTVSSNYMSWVRQAPGKGLEWVSVIYSGGSTYYADSVKGRFTISRHNSKNTLFLQMNSLRAEDTAVYYCARDRPITGTTLDVWGQGTTVTVSS

SEQ ID NO: 325 - protein, 13 aa, synthetic construct
ARDRPITGTTLDV

SEQ ID NO: 326 - protein, 108 aa, synthetic construct
DIQMTQSPSSLFASVGDRVTITCRASQSISSYLNWYQQKPGKAPKLLIYAASSLQSGVPSRFSGSGSGTDFTLTISSLQPEDFAFYYCQQTYSTPPITFGQGTRLEIK

SEQ ID NO: 327 - protein, 10 aa, synthetic construct
QQTYSTPPIT

SEQ ID NO: 328 - protein, 117 aa, synthetic construct
EVQLVESGGGLVQPGGSLRLSCAASGFTVSSNYMNWVRQAPGKGLEWVSVIYSGGSTFYADSVRGRFTISRHNSKNTLYLQMNSLRAEDTAVYYCARDGSAYGMDVWGQGTTVTVSS

SEQ ID NO: 329 - protein, 11 aa, synthetic construct
ARDGSAYGMDV

SEQ ID NO: 330-339 - skipped (10 entries)

SEQ ID NO: 340 - protein, 108 aa, synthetic construct
DIQMTQSPSSLSASVGDRVTITCRASQSISSFLNWYQQKPGKAPKLLIYAASSLQSGVPSRFSGSGSGTDFTLTISSLQPEDFATYYCQQSYSSPPITFGQGTRLEIK

SEQ ID NO: 341 - protein, 6 aa, synthetic construct
QSISSF

SEQ ID NO: 342 - protein, 10 aa, synthetic construct
QQSYSSPPIT

SEQ ID NO: 343 - protein, 115 aa, synthetic construct
EVQLVESGGGLVQPGGSLRLSCAASGFTVSSNYMSWVRQAPGKGLEWVSVIYSGGSTFYADSVKGRFTISRHNSKNTLYLQMNSLRAEDTAVYYCAREGYGMDVWGQGTTVTVSS

SEQ ID NO: 344 - protein, 9 aa, synthetic construct
AREGYGMDV

SEQ ID NO: 345 - protein, 120 aa, synthetic construct
QVQLVESGGGVVQPGRSLRLSCVASGFTFSIYGMDWVRQAPGKGLEWVAVISYDGSNKYYADSVKGRFTISRDNSKNTLYLQMNSLRPEDTAVYYCAKDREAAADAFDIWGQGTMVTVSS

SEQ ID NO: 346 - protein, 8 aa, synthetic construct
GFTFSIYG

SEQ ID NO: 347 - protein, 13 aa, synthetic construct
AKDREAAADAFDI

SEQ ID NO: 348 - protein, 107 aa, synthetic construct
DIQMTQSPSSVSASVGDRVTITCRASQGISSWLAWYQQKPGKAPKLLIYAASSLHSGVPSRFSGSGSGTDFTLTISSLQPEDFATYYCQQANSFRLTFGGGTKVEIK

SEQ ID NO: 349 - protein, 9 aa, synthetic construct
QQANSFRLT

SEQ ID NO: 350 - protein, 116 aa, synthetic construct
EVQLVESGGGLVQPGGSLRLSCAASGFIVSRNYMSWVRQAPGKGLEWVSVIYSGGSTFYADSVKGRFTISRHNSKNTLYLQMNSLKAEDTAVYYCARELRGYFDYWGQGTLVTVSS

SEQ ID NO: 351 - protein, 8 aa, synthetic construct
GFIVSRNY

SEQ ID NO: 352 - protein, 10 aa, synthetic construct
ARELRGYFDY

SEQ ID NO: 353 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQLLNSYPYTFGQGTKLEIK

SEQ ID NO: 354 - protein, 9 aa, synthetic construct
QLLNSYPYT

SEQ ID NO: 355 - protein, 117 aa, synthetic construct
EVQLVESGGGLVQPGGSLRLSCAASGFTVSSNYMTWVRQAPGKGLEWVSVIYSGGSTFYADSVKGRFTISRHNSENTLYLQMNSLRAEDTAVYYCARDLAAAGTDYWGQGALVTVSS

SEQ ID NO: 356 - protein, 11 aa, synthetic construct
ARDLAAAGTDY

SEQ ID NO: 357 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPDDFATYYCQHLNSYLYTFGQGTKLEIK

SEQ ID NO: 358 - protein, 9 aa, synthetic construct
QHLNSYLYT

SEQ ID NO: 359 - protein, 120 aa, synthetic construct
QVQLVESGGGVVQPGRSLRLSCAASGFTFSSYGMHWVRQAPGKGLEWVAVISYDGSYKYYADSVKGRFTISRDNSKNTLYLQMNSLRAEDTAVYYCAIDGGTVTTIFDYWGQGTLVTVSS

SEQ ID NO: 360 - protein, 8 aa, synthetic construct
GFTFSSYG

SEQ ID NO: 361 - protein, 8 aa, synthetic construct
ISYDGSYK

SEQ ID NO: 362 - protein, 13 aa, synthetic construct
AIDGGTVTTIFDY

SEQ ID NO: 363 - protein, 108 aa, synthetic construct
DIQMTQSPSSLSASVGDRVTITCRASQSISSYLNWYQQKPGKAPKLLIYTASSLQSGVPSRFSGSGSGTDFTLTISSLQPEDFATYYCQQSYSTPPWTFGQGTKVEIK

SEQ ID NO: 364 - skipped

SEQ ID NO: 365 - protein, 10 aa, synthetic construct
QQSYSTPPWT

SEQ ID NO: 366 - protein, 125 aa, synthetic construct
EVQLVETGGGLIQPGESLRLSCAASGFIVSTNYMNWVRQAPGKGLEWVSVIYRGGSTYYADSVKGRFTISRDNSKNTLFLQMNSLRAEDTAVYYCARGHEDFDYDTPASGAFDIWGQGTMVTVSS

SEQ ID NO: 367 - protein, 8 aa, synthetic construct
GFIVSTNY

SEQ ID NO: 368 - protein, 7 aa, synthetic construct
IYRGGST

SEQ ID NO: 369 - protein, 19 aa, synthetic construct
ARGHEDFDYDTPASGAFDI

SEQ ID NO: 370 - protein, 110 aa, synthetic construct
QSALTQPPSASGSPGQSVTISCTGTSSDVGGHNYVSWYQQHPGKAPKVMIYEVSKRPSGVPDRFSGSKSGNTASLTVSGLQAEDEADYYCSSYAGSNKFVFGTGTKVTVL

SEQ ID NO: 371 - protein, 9 aa, synthetic construct
SSDVGGHNY

SEQ ID NO: 372 - skipped

SEQ ID NO: 373 - protein, 10 aa, synthetic construct
SSYAGSNKFV

SEQ ID NO: 374 - protein, 123 aa, synthetic construct
EVQLVESGGGLVQPGGSLRLSCAASGFAVSSNYMTWVRQAPGKGLEWVATIYSGDSTYYADSVKGRFTISRHNSKNTLSLQMNSLRADDTAIYYCARLSGVLSGTYNGAFDIWGQGTMVTVSS

SEQ ID NO: 375 - protein, 8 aa, synthetic construct
GFAVSSNY

SEQ ID NO: 376 - protein, 7 aa, synthetic construct
IYSGDST

SEQ ID NO: 377 - protein, 17 aa, synthetic construct
ARLSGVLSGTYNGAFDI

SEQ ID NO: 378 - protein, 110 aa, synthetic construct
QSALTQPASVSGSPGQSITISCTGTSSDFGNYNIVSWYQQHPGQAPKVMIYEGNKRPSGVSNRFSGSKSGHTASLTISGLQAEDEADYYCSSYAGSSTWVFGGGTKLTVL

SEQ ID NO: 379 - protein, 9 aa, synthetic construct
SSDFGNYNI

SEQ ID NO: 380 - skipped

SEQ ID NO: 381 - protein, 10 aa, synthetic construct
SSYAGSSTWV

SEQ ID NO: 382 - protein, 122 aa, synthetic construct
EVQLVESGGGLVQPGGSLRLSCAASGFTVSSNYMSWVRQAPGKGLEWVSAIYSGDSTYYADSVKGRFTISKDNSKNTLYLQMNSLRGEDTAVYYCARLGGALQAYDGAFDYWGQGTLATVSS

SEQ ID NO: 383 - protein, 8 aa, synthetic construct
GFTVSSNY

SEQ ID NO: 384 - protein, 16 aa, synthetic construct
ARLGGALQAYDGAFDY

SEQ ID NO: 385 - protein, 110 aa, synthetic construct
QSALTQPASVSGSPGQSITISCTGTSSDFGSYNLVSWYQQHPGKAPKLMIYEGNKRPSGVSIRFSGSKSGNTASLTISGLQAEDEADYYCSSYAGSSTWVFGGGTKLTVL

SEQ ID NO: 386 - protein, 9 aa, synthetic construct
SSDFGSYNL

SEQ ID NO: 387 - protein, 121 aa, synthetic construct
EVQLVESGGGLVQPGGSLRLSCAASGFTVTSNYMSWVRQAPGKGLEWVSAIYSGESTYYADSVKGRFTISRDSSKNTLYLQMNSLRVEDTAVYYCASIHDYSNYVGGFDPWGQGTLVTVSS

SEQ ID NO: 388 - protein, 8 aa, synthetic construct
GFTVTSNY

SEQ ID NO: 389 - protein, 7 aa, synthetic construct
IYSGEST

SEQ ID NO: 390 - protein, 15 aa, synthetic construct
ASIHDYSNYVGGFDP

SEQ ID NO: 391 - protein, 110 aa, synthetic construct
QSALTQPASVSGSPGQSITISCTGPSSDFGSYNLVSWYQQHPGKAPKLMIYEGTKRPSGVSNRFSGSKSGNTASLTISGLQAEDEADYYCSSYAGSSTWVFGGGTKLTVL

SEQ ID NO: 392 - skipped

SEQ ID NO: 393 - protein, 122 aa, synthetic construct
QVQLQESGPGLVKPSETLSLTCTVSGGSISGYYWSWIRQPPGKGLEWIGYIYYSGSTNYNPSLKSRVTMSVDTSKNQFSLKLSSVTAADTAVYYCARYPLYSESSGYHFDYWGHGTLVTVSS

SEQ ID NO: 394 - protein, 8 aa, synthetic construct
GGSISGYY

SEQ ID NO: 395 - protein, 7 aa, synthetic construct
IYYSGST

SEQ ID NO: 396 - protein, 16 aa, synthetic construct
ARYPLYSESSGYHFDY

SEQ ID NO: 397 - protein, 110 aa, synthetic construct
QSALTQPASVSGSPGQSITISCTGTSSDVGSYNLVSWYQQDPGKAPKLMLYEDSKRPSGVSNRFSGSKSGNTASLTISGLQAEDEADYYCSSYAGRSTWVFGGGTKLTVL

SEQ ID NO: 398 - protein, 9 aa, synthetic construct
SSDVGSYNL

SEQ ID NO: 399 - skipped

SEQ ID NO: 400 - protein, 10 aa, synthetic construct
SSYAGRSTWV

SEQ ID NO: 401 - protein, 117 aa, synthetic construct
EVQLVESGGGLVQPGGSLRLSCAATGITVSSNYMSWVRQAPGKGLEWVSLIYSGGSTFYADSVKGRFTISRHNSKNTLYLQMSSLRAEDTAVYYCARDLVDFGMDVWGQGTTVTVSS

SEQ ID NO: 402 - protein, 8 aa, synthetic construct
GITVSSNY

SEQ ID NO: 403 - protein, 7 aa, synthetic construct
IYSGGST

SEQ ID NO: 404 - protein, 11 aa, synthetic construct
ARDLVDFGMDV

SEQ ID NO: 405 - protein, 107 aa, synthetic construct
DIQLTQSPSSLSASVGDRVTITCRASQGISNYLAWYQQKPGKAPKLLIYAASTLQSGVPSRFSGSGSGTDFTLTISSLQPEDFGTYYCQQLNSDSPTFGQGTKVEIK

SEQ ID NO: 406 - protein, 6 aa, synthetic construct
QGISNY

SEQ ID NO: 407 - skipped

SEQ ID NO: 408 - protein, 9 aa, synthetic construct
QQLNSDSPT

SEQ ID NO: 409 - protein, 128 aa, synthetic construct
EVQLVQSGAEVKKPGESLKISCKGSGYSFTSYWIGWVRQMPGKGLEWMGIIYPGDTNTRYSPSFQGQVTISADKSISTAYLQWSSLKASDTAMYYCARLRAPSGPWEGAIPGGGMDVWGQGTTVTVSS

SEQ ID NO: 410 - protein, 8 aa, synthetic construct
GYSFTSYW

SEQ ID NO: 411 - protein, 8 aa, synthetic construct
IYPGDTNT

SEQ ID NO: 412 - protein, 21 aa, synthetic construct
ARLRAPSGPWEGAIPGGGMDV

SEQ ID NO: 413 - protein, 108 aa, synthetic construct
EIVMTQSPATLSVSPGERATLSCRASQSVSSNLAWYQQKHGQAPRLLIYGASTRATGIPARFSGSGSGTEFTLTISSLQSEDFAVYYCQQYNNWPPFTFGPGTKVDIK

SEQ ID NO: 414 - protein, 6 aa, synthetic construct
QSVSSN

SEQ ID NO: 415-416 - skipped (2 entries)

SEQ ID NO: 417 - protein, 10 aa, synthetic construct
QQYNNWPPFT

SEQ ID NO: 418 - protein, 127 aa, synthetic construct
QVQLVQSGAEVKKPGASVKVSCKASGYTFTDYYMHWVRQAPGQGLEWMGWINPNSGGTNYAQKFQGRVTMTRDTSISTAYMELSRLRSDDTAIYYCARTLFKYYDILIGYGEYFQHWGQGTLVIVSS

SEQ ID NO: 419 - protein, 8 aa, synthetic construct
GYTFTDYY

SEQ ID NO: 420 - protein, 8 aa, synthetic construct
INPNSGGT

SEQ ID NO: 421 - protein, 20 aa, synthetic construct
ARTLFKYYDILIGYGEYFQH

SEQ ID NO: 422 - protein, 108 aa, synthetic construct
EIVLTQSPGTLSLSPGERATLSCRASQSVSSSYLAWYQQKPGQAPRLLIYGASSRATAIPDRFSGSGSGTDFTLTISRLEPEDFAVYFCQQYGSSPWTFGQGTKVEIK

SEQ ID NO: 423 - protein, 7 aa, synthetic construct
QSVSSSY

SEQ ID NO: 424 - protein, 9 aa, synthetic construct
QQYGSSPWT

SEQ ID NO: 425 - protein, 120 aa, synthetic construct
EVQLVESGGGLVQPGGSLRLSCAASGFTVSRSYMTWVRQAPGKGLEWVSVIFTGGRTDHADSVKGRFTISRHNSKNTLYLQMNSLRTEDTAVYYCARVAIVGATGAFDIWGQGTMVTVSS

SEQ ID NO: 426 - protein, 8 aa, synthetic construct
GFTVSRSY

SEQ ID NO: 427 - protein, 7 aa, synthetic construct
IFTGGRT

SEQ ID NO: 428 - protein, 14 aa, synthetic construct
ARVAIVGATGAFDI

SEQ ID NO: 429 - protein, 112 aa, synthetic construct
QSVLTQPPSVSGAPGQRVTISCTGSSSNIGAGDDVHWYQQLPGTAPKLLIYGNSNRPSGVPDRFSGSKSGTSASLAITGLQAEDEADYYCQSYDSSLSGSVVFGGGTKLTVL

SEQ ID NO: 430 - protein, 9 aa, synthetic construct
SSNIGAGDD

SEQ ID NO: 431 - skipped

SEQ ID NO: 432 - protein, 12 aa, synthetic construct
QSYDSSLSGSVV

SEQ ID NO: 433 - protein, 123 aa, synthetic construct
EVQLVESGGGLVQPGRSLRLSCVASGFTFDDSAMHWVRQPPGKGLEWVSGISWNSGIIGYAESVKGRFIISRDNAKNSLFLHMRGLRPEDTAVYYCARDRLELSYYYYGMDVWGQGTTVTVSS

SEQ ID NO: 434 - protein, 8 aa, synthetic construct
GFTFDDSA

SEQ ID NO: 435 - protein, 8 aa, synthetic construct
ISWNSGII

SEQ ID NO: 436 - protein, 16 aa, synthetic construct
ARDRLELSYYYYGMDV

SEQ ID NO: 437 - protein, 110 aa, synthetic construct
QSVLTQPPSASGTPGQRVTISCSGSSSNIGSNTVNWYQQFPGTAPRLLIYSNDQRPSGIPDRFSGSKSGTSASLAISGLQSEDEADYYCSTWDDSLNGAVFGGGTQLTVL

SEQ ID NO: 438 - protein, 8 aa, synthetic construct
SSNIGSNT

SEQ ID NO: 439 - skipped

SEQ ID NO: 440 - protein, 11 aa, synthetic construct
STWDDSLNGAV

SEQ ID NO: 441 - protein, 121 aa, synthetic construct
RVTLRESGPALVKPTQTLTLTCTFSGFSLYSSGMSVSWIRQPPGKALEWLARIDWDDDEYYSSFLKTRLTISKDISKNQVVLTMTNLDPVDTATYYCVRPRYSSNFGFEYWGQGTLVTVSS

SEQ ID NO: 442 - protein, 10 aa, synthetic construct
GFSLYSSGMS

SEQ ID NO: 443 - protein, 7 aa, synthetic construct
IDWDDDE

SEQ ID NO: 444 - protein, 13 aa, synthetic construct
VRPRYSSNFGFEY

SEQ ID NO: 445 - protein, 110 aa, synthetic construct
QSVLTQPPSVSAAPGQKVTISCSGSISNIGNNYVSWYQQLPGTAPKLLIYDNDKRPSGIPDRFSGSRSGTSATLGITGLQTGDEADYYCGTWDSSLTAYVFGTGTKVTVL

SEQ ID NO: 446 - protein, 8 aa, synthetic construct
ISNIGNNY

SEQ ID NO: 447 - skipped

SEQ ID NO: 448 - protein, 11 aa, synthetic construct
GTWDSSLTAYV

SEQ ID NO: 449 - protein, 121 aa, synthetic construct
QVQLVQSGAEVKKPGSSVKVSCKASGDTFSTYAINWVRQAPGQGLEWMGRFIHIFGTANYAQKFQGRVTITADESTSTAYMELRSLRSEDTAVYYCARDGVDYGDYRPDYWGQGTLVTVSS

SEQ ID NO: 450 - protein, 8 aa, synthetic construct
GDTFSTYA

SEQ ID NO: 451 - protein, 8 aa, synthetic construct
FIHIFGTA

SEQ ID NO: 452 - protein, 14 aa, synthetic construct
ARDGVDYGDYRPDY

SEQ ID NO: 453 - protein, 108 aa, synthetic construct
EIVLTQSPGTLSLSPGERATLSCRASQSVSSNYLAWYQQKPGQAPRLLIYGASSRATGIPERFSGSGSGTDFTLTISRLEPEDFAVYYCQQYGSSLYTFGQGTKLEIK

SEQ ID NO: 454 - protein, 7 aa, synthetic construct
QSVSSNY

SEQ ID NO: 455 - protein, 9 aa, synthetic construct
QQYGSSLYT

SEQ ID NO: 456 - protein, 119 aa, synthetic construct
QVQLVQSGAEVKKPGSSVKVSCKAPGGTFSTYAINWVRQAPGQGLEWMGRIVPFFGTTNYAQKFQGRVTITADESTSTAYMELSSLTSEDTAVYYCARGPRPFGELLYWGQGTLVTVSS

SEQ ID NO: 457 - protein, 8 aa, synthetic construct
GGTFSTYA

SEQ ID NO: 458 - protein, 8 aa, synthetic construct
IVPFFGTT

SEQ ID NO: 459 - protein, 12 aa, synthetic construct
ARGPRPFGELLY

SEQ ID NO: 460 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQAEDIATYYCQQYDNLLFTFGPGTKVDIK

SEQ ID NO: 461 - protein, 6 aa, synthetic construct
QDISNY

SEQ ID NO: 462 - skipped

SEQ ID NO: 463 - protein, 9 aa, synthetic construct
QQYDNLLFT

SEQ ID NO: 464 - protein, 119 aa, synthetic construct
EVQLVETGGGLIQPGGSLRLSCAAPGFIVSSNYMSWVRQAPGKGLEWVSLLYPGGSTYYADSVKGRFTISRDNSKNTLYLQMNSLRADDTAVYYCARVLPIVEDHFDYWGQGTLVTVSS

SEQ ID NO: 465 - protein, 8 aa, synthetic construct
GFIVSSNY

SEQ ID NO: 466 - protein, 7 aa, synthetic construct
LYPGGST

SEQ ID NO: 467 - protein, 13 aa, synthetic construct
ARVLPIVEDHFDY

SEQ ID NO: 468 - protein, 110 aa, synthetic construct
DIQLTQSPSFLSASVGDRVTITCRASQGMSSYLAWYQKKPGKAPKLLIYGATILQSGVPSRFSGSGSGTEFTLTISSLQPEDFATYYCQQLNSDPLYMYTFGQGTKLEIK

SEQ ID NO: 469 - protein, 6 aa, synthetic construct
QGMSSY

SEQ ID NO: 470 - skipped

SEQ ID NO: 471 - protein, 12 aa, synthetic construct
QQLNSDPLYMYT

SEQ ID NO: 472 - protein, 122 aa, synthetic construct
QVQLVESGGGVVQPGRSLRLSCAASGFVFNSYGMHWVRQAPGKGLEWVAVLWYEGSKNYYADSVKGRFTISRDNSKNTLYLQMNSLRAEDTAVYYCARHGSGSFFGYYLDYWGQGTLVTVSS

SEQ ID NO: 473 - protein, 8 aa, synthetic construct
GFVFNSYG

SEQ ID NO: 474 - protein, 8 aa, synthetic construct
LWYEGSKN

SEQ ID NO: 475 - protein, 15 aa, synthetic construct
ARHGSGSFFGYYLDY

SEQ ID NO: 476 - protein, 107 aa, synthetic construct
DIQMTQSPSSLSASVGDRVTITCRASHNINDFLNWYQQKPGKAPRLLIYAASSLQSGVPSRFSGSGSGTDFTLTISSLQPEDFATYYCQESYTTPPTFGQGTKLEIK

SEQ ID NO: 477 - protein, 6 aa, synthetic construct
HNINDF

SEQ ID NO: 478 - protein, 9 aa, synthetic construct
QESYTTPPT

SEQ ID NO: 479 - protein, 122 aa, synthetic construct
QLQLQESGPGLVKSSETLSLTCTVSASITGSTYYWGWIRQPPGKGLEWIGSIYYSGTTYYKASLKSRVTISVDTSKNQFSLKLTSVTVADTAVYYCVSHLLGNYHKYGMDVWGQGTTVTVSS

SEQ ID NO: 480 - protein, 9 aa, synthetic construct
ASITGSTYY

SEQ ID NO: 481 - protein, 7 aa, synthetic construct
IYYSGTT

SEQ ID NO: 482 - protein, 15 aa, synthetic construct
VSHLLGNYHKYGMDV

SEQ ID NO: 483 - protein, 112 aa, synthetic construct
QSVLTQPPSASGTPGQRVTISCSGSRSNIGINTVNWYQQFPGTALNLLIFSDNQRPSGVPDRFSGSKSGTSASLAISGLQSEDEADYYCAAWDDSLNGPDWVFGGGTKLTVL

SEQ ID NO: 484 - protein, 8 aa, synthetic construct
RSNIGINT

SEQ ID NO: 485 - skipped

SEQ ID NO: 486 - protein, 13 aa, synthetic construct
AAWDDSLNGPDWV

SEQ ID NO: 487 - protein, 123 aa, synthetic construct
EVQLVESGGALVQPGGSLRLSCAASGFPFTIYAMTWVRQAPGKGLEWVSVITGSGDSTYYADSVKGRFTISRDNSKNTLYLQMNSLRAEDTAVYYCVKTGEGFFYYYYGMDVWGQGTTVTVSS

SEQ ID NO: 488 - protein, 8 aa, synthetic construct
GFPFTIYA

SEQ ID NO: 489 - protein, 8 aa, synthetic construct
ITGSGDST

SEQ ID NO: 490 - protein, 16 aa, synthetic construct
VKTGEGFFYYYYGMDV

SEQ ID NO: 491 - protein, 110 aa, synthetic construct
QSVLTQPPSVSAAPGQKVTISCSGSSSNIGNSYISWYQQLPGTAPKLLIYVNNKRPSGIPDRFSGSKSGTSATLGITGLQTGDEADYYCGTWDSSLSAGVFGSGTKVTVL

SEQ ID NO: 492 - protein, 8 aa, synthetic construct
SSNIGNSY

SEQ ID NO: 493 - skipped

SEQ ID NO: 494 - protein, 11 aa, synthetic construct
GTWDSSLSAGV

SEQ ID NO: 495 - protein, 121 aa, synthetic construct
EVQLVESGGGLVQPGGSLRLSCAASGFSFSSNWMSWVRQAPGKGLEWVGNINQDGSEKYYVDSVEGRFTISRDNAKKSLYLQMNSLRAEDTAVYYCARDLGVLWFGDFVDWGQGALVTVSS

SEQ ID NO: 496 - protein, 8 aa, synthetic construct
GFSFSSNW

SEQ ID NO: 497 - protein, 8 aa, synthetic construct
INQDGSEK

SEQ ID NO: 498 - protein, 14 aa, synthetic construct
ARDLGVLWFGDFVD

SEQ ID NO: 499 - protein, 107 aa, synthetic construct
DIQMTQSPSSLSASVGDRVTITCRASQAISNSLAWYQQKPGKAPKLLLYAASTLESGVPSRFSGSGSGTDYTLTISSLQPEDFATYYCLQYYGSPRTFGQGTKVEIK

SEQ ID NO: 500 - protein, 6 aa, synthetic construct
QAISNS

SEQ ID NO: 501 - protein, 9 aa, synthetic construct
LQYYGSPRT

SEQ ID NO: 502 - protein, 11 aa, synthetic construct
ARDLVVYGMDV

SEQ ID NO: 503 - protein, 107 aa, synthetic construct
DIQLTQSPSSLSASVGDRVTITCRASQGISSYLAWYQQKPGKAPKLLIYAASTLQSGVPSRFSGSGSGTDFTLTISSLQPEDFATYYCQHLNSDPHTFGQGTKLEIK

SEQ ID NO: 504 - protein, 6 aa, synthetic construct
QGISSY

SEQ ID NO: 505 - protein, 9 aa, synthetic construct
QHLNSDPHT

SEQ ID NO: 506 - protein, 123 aa, synthetic construct
EVQLVESGGGLVQPGGSLRLSCSASGFTFSRYAMYWVRQAPGKGLEYVSAISSDGGSTYDADSVKGRFTISRANSKNTLYLQMSSLRAEDTAVYYCVKGLRELLYYYYGMDVWGQGTTVTVSS

SEQ ID NO: 507 - protein, 8 aa, synthetic construct
GFTFSRYA

SEQ ID NO: 508 - protein, 8 aa, synthetic construct
ISSDGGST

SEQ ID NO: 509 - protein, 16 aa, synthetic construct
VKGLRELLYYYYGMDV

SEQ ID NO: 510 - protein, 107 aa, synthetic construct
DIQMTQSPSSLSASVGDRVTITCRAGQSISSFLNWYQQKPGKAPKLLIYAASSLQSGVPSRFSGSGSGTDFTLTISSLQPEDFATYYCQQSYITPFTFGPGTKVDIK

SEQ ID NO: 511 - protein, 6 aa, synthetic construct
QSISSF

SEQ ID NO: 512 - protein, 9 aa, synthetic construct
QQSYITPFT

SEQ ID NO: 513 - protein, 120 aa, synthetic construct
EVQLVESGGGLVQPGGSLRLSCAASGFTFSSYEMNWVRQAPGKGLEWVSYISSSGSILYYADSVKGRFTASRDNAKNSLYLQMNSLRAEDTGVYYCARREYQLLRPFEFWGQGTLVTVSS

SEQ ID NO: 514 - protein, 8 aa, synthetic construct
GFTFSSYE

SEQ ID NO: 515 - protein, 8 aa, synthetic construct
ISSSGSIL

SEQ ID NO: 516 - protein, 13 aa, synthetic construct
ARREYQLLRPFEF

SEQ ID NO: 517 - protein, 108 aa, synthetic construct
DIQMTQSPSTLSASVGDRVTITCRASQSISSWLAWYQQKSGKAPKLLIYKASSLESGVPSRFSGSGSGTEFTLTISSLQPDDFATYYCQQYDSYSPWTFGQGTKVEIK

SEQ ID NO: 518 - protein, 6 aa, synthetic construct
QSISSW

SEQ ID NO: 519 - skipped

SEQ ID NO: 520 - protein, 10 aa, synthetic construct
QQYDSYSPWT

SEQ ID NO: 521 - protein, 116 aa, synthetic construct
EVQLVETGGGLIQPGGSLRLSCAASEFIVSRNYMSWVRQAPGKGLEWVSLIYSGGSTYYADSVKGRFTISRDNSKNILYLQMNSLRAEDTAVYYCARDRGGISDYWGQGTLVTVSS

SEQ ID NO: 522 - protein, 8 aa, synthetic construct
EFIVSRNY

SEQ ID NO: 523 - protein, 10 aa, synthetic construct
ARDRGGISDY

SEQ ID NO: 524 - protein, 107 aa, synthetic construct
DIQMTQSPSSLSASVGDRVTITCQASQDINKYLNWFQQKPGKAPKLLIYDASNLETGVPSRFSGSGSGTDFTFTISSLQPEDIATYYCQQYDNLPRTFGQGTKLEIK

SEQ ID NO: 525 - protein, 6 aa, synthetic construct
QDINKY

SEQ ID NO: 526 - protein, 9 aa, synthetic construct
QQYDNLPRT

SEQ ID NO: 527 - protein, 122 aa, synthetic construct
QVQLVQSGAEVKKPGASVRVSCKASGYTFTGNYIHWVRQAPGQGLEWMAWINPNSGGKNYAQKFQGRVTLTRDTSVNTAYMELSRLRSDDTAMYYCARDVNYNILTGPGGLWGQGTLVTVSS

SEQ ID NO: 528 - protein, 8 aa, synthetic construct
GYTFTGNY

SEQ ID NO: 529 - protein, 8 aa, synthetic construct
INPNSGGK

SEQ ID NO: 530 - protein, 15 aa, synthetic construct
ARDVNYNILTGPGGL

SEQ ID NO: 531 - protein, 110 aa, synthetic construct
QSALTQPPSASGSPGQSVTISCTGTSSDVGGYNFVSWYQQHPGKAPKLMIYEVSKRPSGVPDRFSGSKSGNTASLTVSGLQAEDEADYYCSSYAGSNNFVFGTGTKVTVL

SEQ ID NO: 532 - protein, 9 aa, synthetic construct
SSDVGGYNF

SEQ ID NO: 533 - protein, 10 aa, synthetic construct
SSYAGSNNFV

SEQ ID NO: 534 - protein, 122 aa, synthetic construct
QVQLVQSGADVKKPGASVKVSCKTSGYIFTGYYIHWVRQAPGQGLEWMGWINPNSGGTNSAQKFQGRVTMTRDTSISTVYMELSRLRSDDTAVYYCAKSNSFSVLDYNFDYWGQGTLVTVSS

SEQ ID NO: 535 - protein, 8 aa, synthetic construct
GYIFTGYY

SEQ ID NO: 536 - protein, 15 aa, synthetic construct
AKSNSFSVLDYNFDY

SEQ ID NO: 537 - protein, 109 aa, synthetic construct
SSELTQDPAVSVALGQTVRITCQGDSLRSYYASWYQQKPGQAPVLVIYDKNNRPSGIPDRFSGSSSGNTASLTITGAQAEDEADYYCNSRDSSRNHLVVFGGGTKLTVL

SEQ ID NO: 538 - protein, 6 aa, synthetic construct
SLRSYY

SEQ ID NO: 539 - skipped

SEQ ID NO: 540 - protein, 12 aa, synthetic construct
NSRDSSRNHLVV

SEQ ID NO: 541 - protein, 122 aa, synthetic construct
QVQLVQSGAEVKKPGASVKVSCKASGYIFTDHYMHWVRQAPGQGLEWMGWINPNSGATNYAQNFQGRVTMTRDTSIITVYMELSRLRSDDTAVYYCARDSSPYMIRYTMDVWGQGTTITVSS

SEQ ID NO: 542 - protein, 8 aa, synthetic construct
GYIFTDHY

SEQ ID NO: 543 - protein, 8 aa, synthetic construct
INPNSGAT

SEQ ID NO: 544 - protein, 15 aa, synthetic construct
ARDSSPYMIRYTMDV

SEQ ID NO: 545 - protein, 111 aa, synthetic construct
QSALTQPASVSGSPGQSITISCTGTSSDVGSYNLVSWYQQHPGKAPKLMIYELTKRPSGVSNRFSGSKSGNTASLTISGLQAEDEADYYCSSYRGSVTDVVFGGGTKLTVL

SEQ ID NO: 546 - skipped

SEQ ID NO: 547 - protein, 11 aa, synthetic construct
SSYRGSVTDVV

SEQ ID NO: 548 - protein, 118 aa, synthetic construct
QVQLQESGPGLVKPSGTLSLTCDVSGGSISSNNWWSWVRQRPGKGLQWIGEIYHNENTNYNPSLKSRVIMSIDKSKNQFSLKLTSVTAADTAVYYCARGDVLDWFDPWGQGTLVTVSS

SEQ ID NO: 549 - protein, 9 aa, synthetic construct
GGSISSNNW

SEQ ID NO: 550 - protein, 7 aa, synthetic construct
IYHNENT

SEQ ID NO: 551 - protein, 11 aa, synthetic construct
ARGDVLDWFDP

SEQ ID NO: 552 - protein, 110 aa, synthetic construct
QSALTQPASVSGSPGQSTTISCTGTSSDVGGYNYVSWYQQYPGKAPKLMIYDVTNRPSGVSHRFSGSKSGNTASLTISGLQAEDEADYYCSSYTSSSTVIFGGGTKLTVL

SEQ ID NO: 553 - protein, 9 aa, synthetic construct
SSDVGGYNY

SEQ ID NO: 554 - skipped

SEQ ID NO: 555 - protein, 10 aa, synthetic construct
SSYTSSSTVI

SEQ ID NO: 556 - protein, 119 aa, synthetic construct
QITLKESGPTLVKPTQTLTLTCTFSGFSFSTSGVGVGWIRQPPGKTLEWLALIYWDDDKRYSPSLKSRLTITKDTSKNQVVLTMTNMDPVDTATYFCAHHGIPTIFGYWGQGALVTVSS

SEQ ID NO: 557 - protein, 10 aa, synthetic construct
GFSFSTSGVG

SEQ ID NO: 558 - protein, 7 aa, synthetic construct
IYWDDDK

SEQ ID NO: 559 - protein, 11 aa, synthetic construct
AHHGIPTIFGY

SEQ ID NO: 560 - protein, 109 aa, synthetic construct
QSALTQPASVSGSPGQSITISCTGTSSDLGVFNYVSWYQQHPGKAPKLMIYEVTNRPSGVSNRFSGSKSGNTASLTISGLQAEDEADYYCSSYTTSSTVFGGGTKLTVL

SEQ ID NO: 561 - protein, 9 aa, synthetic construct
SSDLGVFNY

SEQ ID NO: 562 - skipped

SEQ ID NO: 563 - protein, 9 aa, synthetic construct
SSYTTSSTV

SEQ ID NO: 564 - protein, 120 aa, synthetic construct
QVQLVQSGAEVKKPGASVKVSCKASGYTFTGYFIHWVRQAPGQGLEWMGWISPISGGTNYAQKFQGRVTVTRDTSITTAYMELSRLTSDDTAVYHCAREGGTWKGDFDYWGQGTLVTVSS

SEQ ID NO: 565 - protein, 8 aa, synthetic construct
GYTFTGYF

SEQ ID NO: 566 - protein, 8 aa, synthetic construct
ISPISGGT

SEQ ID NO: 567 - protein, 13 aa, synthetic construct
AREGGTWKGDFDY

SEQ ID NO: 568 - protein, 110 aa, synthetic construct
QSALTQPASVSGSPGQSITISCTGTSSDVESYNLVSWYQQHPGKAPKLMIYEGSKRPSGVSNRFSGSKSGNTASLTISGLQAEDEADYYCSSYAGSSTWVFGGGTKLTVL

SEQ ID NO: 569 - protein, 9 aa, synthetic construct
SSDVESYNL

SEQ ID NO: 570 - skipped

SEQ ID NO: 571 - protein, 122 aa, synthetic construct
QVQLVQSGAEVKKPGASVKVSCKASGYTFTDYFIHWVRQAPGQGLEWMGWINPTSGGTNYAQKFRGRVTMTRDTSISTAYVELSRLRSDDTALYYCARDLPFYSDTKQFDYWGQGTLVTVSS

SEQ ID NO: 572 - protein, 8 aa, synthetic construct
GYTFTDYF

SEQ ID NO: 573 - protein, 8 aa, synthetic construct
INPTSGGT

SEQ ID NO: 574 - protein, 15 aa, synthetic construct
ARDLPFYSDTKQFDY

SEQ ID NO: 575 - protein, 110 aa, synthetic construct
QSALTQPASVSGYPGQSITISCTGTSSDVGGYNYVSWYQQHPGKAPKLMIFDVSNRPSGVSNRFSGSKSGNTASLTFSGLQAEDEADYYCSSYTSSSTLVFGGGTKLTVL

SEQ ID NO: 576 - skipped

SEQ ID NO: 577 - protein, 10 aa, synthetic construct
SSYTSSSTLV

SEQ ID NO: 578 - protein, 120 aa, synthetic construct
QVQLVQSGAEVKKPGSSVKVSCKASGGTFSNNAISWVRQAPGQGLEWMGKIIPILGIGGDAQKFQGRFTITADKSTSTAYMELSSLRSEDTAVYYCANSGDSRPHAFDIWGQGTMVTVSS

SEQ ID NO: 579 - protein, 8 aa, synthetic construct
GGTFSNNA

SEQ ID NO: 580 - protein, 8 aa, synthetic construct
IIPILGIG

SEQ ID NO: 581 - protein, 13 aa, synthetic construct
ANSGDSRPHAFDI

SEQ ID NO: 582 - protein, 110 aa, synthetic construct
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSRREVFGGGTKLTVL

SEQ ID NO: 583 - protein, 10 aa, synthetic construct
SSYAGSRREV

SEQ ID NO: 584 - protein, 126 aa, synthetic construct
EVQLVESGGGLVQPGGSLRLSCAASGFTLSSYDMHWVRQVIGKGLEWVSTIGTSGDTYYPGSVKGRFTISRENAKNSLYLQMNSLRAGDTAVYYCARGGVPTTIWSGPYYYYMDVWGKGTTVTVSS

SEQ ID NO: 585 - protein, 8 aa, synthetic construct
GFTLSSYD

SEQ ID NO: 586 - protein, 7 aa, synthetic construct
IGTSGDT

SEQ ID NO: 587 - protein, 20 aa, synthetic construct
ARGGVPTTIWSGPYYYYMDV

SEQ ID NO: 588 - protein, 112 aa, synthetic construct
QSVLTQPPSVSAAPGQKVTISCSGSSSNIGNNYVSWYQQLPGTAPKLLIYENNKRPSGIPDRFSGSKSGTSATLGITGLQTGDEADYYCGTWDSSLSAGQGVFGGGTKLTVL

SEQ ID NO: 589 - protein, 8 aa, synthetic construct
SSNIGNNY

SEQ ID NO: 590 - skipped

SEQ ID NO: 591 - protein, 13 aa, synthetic construct
GTWDSSLSAGQGV

SEQ ID NO: 592 - protein, 118 aa, synthetic construct
QVQLVQSGPEVKKPGASVKVSCKASGYTFTSYAISWVRQAPGQGLEWMGWISAYNGNTNYAQKLQGRVTMTTDTFTSTAYMELRSLRSDDTAVYYCAVPADHITFDYWGQGTLVTVSS

SEQ ID NO: 593 - protein, 8 aa, synthetic construct
GYTFTSYA

SEQ ID NO: 594 - protein, 8 aa, synthetic construct
ISAYNGNT

SEQ ID NO: 595 - protein, 11 aa, synthetic construct
AVPADHITFDY

SEQ ID NO: 596 - protein, 107 aa, synthetic construct
EIVLTQSPGTLSLSPGERATLSCRASQSVSSYLAWYQQKPGQAPRLLIHGASSRATGIPDRFSGSGSGTDFTLTISRLEPEDFAVYYCQQYGSSFLTFGGGTKVEIK

SEQ ID NO: 597 - protein, 6 aa, synthetic construct
QSVSSY

SEQ ID NO: 598 - protein, 9 aa, synthetic construct
QQYGSSFLT

SEQ ID NO: 599 - protein, 122 aa, synthetic construct
QVQLVQSGAEVKKPGSSVKVSCKASGGSFSNYAISWVRQAPGQGLEWMGGIIPFFGTTNYAQKFQGRLTITADESTSTFYMELSSLGSEDTAVYYCAAGYSSGWYPSWFDPWGQGTLVTVSS

SEQ ID NO: 600 - protein, 8 aa, synthetic construct
GGSFSNYA

SEQ ID NO: 601 - protein, 8 aa, synthetic construct
IIPFFGTT

SEQ ID NO: 602 - protein, 15 aa, synthetic construct
AAGYSSGWYPSWFDP

SEQ ID NO: 603 - protein, 108 aa, synthetic construct
EIVLTQSPGTLSLSPGERATLSCRASQSVNNNYLAWYQQKPGQTPRLLIYGASSRATGIPDRFSGSGSGTDFTLTITRLEPEDFAVYHCQQYGRSPTTFGQGTRLEIK

SEQ ID NO: 604 - protein, 7 aa, synthetic construct
QSVNNNY

SEQ ID NO: 605 - protein, 9 aa, synthetic construct
QQYGRSPTT

SEQ ID NO: 606 - protein, 124 aa, synthetic construct
EVQLVQSGAEVKKPGSTMKISCKISGYIFSDYYMHWVQQAPGKGLEWMGLVDPEDGETRYAEKFQGRVTITADTSTDTAYMELSSLRFEDTAVYYCATVGGSSGPWGETLFDHWGQGSLVTVSS

SEQ ID NO: 607 - protein, 8 aa, synthetic construct
GYIFSDYY

SEQ ID NO: 608 - protein, 8 aa, synthetic construct
VDPEDGET

SEQ ID NO: 609 - protein, 17 aa, synthetic construct
ATVGGSSGPWGETLFDH

SEQ ID NO: 610 - protein, 112 aa, synthetic construct
DIVMTQSPLSLPVTPGEPASISCRSSQSLLHSNGYNYINWYLQKPGQSPQLLMYLGSNRASGVPDRFSGSGSGTDSTLKISRVEAEDAGVYYCMQTRQTPYTFGQGTKLEIK

SEQ ID NO: 611 - protein, 11 aa, synthetic construct
QSLLHSNGYNY

SEQ ID NO: 612 - skipped

SEQ ID NO: 613 - protein, 9 aa, synthetic construct
MQTRQTPYT

SEQ ID NO: 614 - protein, 126 aa, synthetic construct
EVQLVESGGGLVQPGGSLRLSCVASGFAFSTYEMNWVRQAPGKGLEWVSYISSSGSTIYYADSVKGRLTISRDNVKNSLYLQMNSLRVEDTAIYYCARDAIRNNDILTDPATFDPWGQGTLVTVSS

SEQ ID NO: 615 - protein, 8 aa, synthetic construct
GFAFSTYE

SEQ ID NO: 616 - protein, 8 aa, synthetic construct
ISSSGSTI

SEQ ID NO: 617 - protein, 19 aa, synthetic construct
ARDAIRNNDILTDPATFDP

SEQ ID NO: 618 - protein, 112 aa, synthetic construct
DIVMTQSPLSLPVTPGEPASISCRSSQSLLYSNGYNYLDWYLQKPGQSPQLLISLGSNRASGVPDRFSGSGSGTDFTLKISRVEAEDVGVYYCMQALQTPYTFGQGTKLEIK

SEQ ID NO: 619 - protein, 11 aa, synthetic construct
QSLLYSNGYNY

SEQ ID NO: 620 - protein, 9 aa, synthetic construct
MQALQTPYT

SEQ ID NO: 621 - protein, 130 aa, synthetic construct
QVQLVQSGAEVKKPGASVKVSCKASGYTFTNYYMHWVRQAPGQGLEWMGIINPSGGSTSYAQKFLGRVTMTRDTSTTTVYMELSSLRSEDTAVYFCARVENLPTKWDLLRKGFESGMDVWGQGTTVTVSS

SEQ ID NO: 622 - protein, 8 aa, synthetic construct
GYTFTNYY

SEQ ID NO: 623 - protein, 8 aa, synthetic construct
INPSGGST

SEQ ID NO: 624 - protein, 23 aa, synthetic construct
ARVENLPTKWDLLRKGFESGMDV

SEQ ID NO: 625 - protein, 107 aa, synthetic construct
EIVMTQSPATVSVSPGERATLSCRASQSVSSNLAWYQQKPGQAPRLLIYGASTRATGIPASFSGSGSGTEFTLTISSLQSEDFAVYYCQQYNNWPITFGPGTKVDIK

SEQ ID NO: 626 - protein, 9 aa, synthetic construct
QQYNNWPIT

SEQ ID NO: 627 - protein, 128 aa, synthetic construct
QLQLQESGPGLVKPSETLSLTCTVSGGSISSSGHYWGWIRQSPGKGLDWIGSIYYSGSTYYNPSLKSRVTISVDTSKNQFSLKLNSVTAADTAVYFCARLEAYCSGDTCHYFYGLDVWGQGTTVTVSS

SEQ ID NO: 628 - protein, 10 aa, synthetic construct
GGSISSSGHY

SEQ ID NO: 629 - protein, 20 aa, synthetic construct
ARLEAYCSGDTCHYFYGLDV

SEQ ID NO: 630 - protein, 107 aa, synthetic construct
DIQMTQSPSSLSASVGDRVTITCRASQSINTYLNWYQQKPGKAPNLLIDGASSLQSGVPSRFSGSGSGTDFILTISSLRPEDFATYYCQQSYSIPLTFGGGTRVEIK

SEQ ID NO: 631 - protein, 6 aa, synthetic construct
QSINTY

SEQ ID NO: 632 - protein, 9 aa, synthetic construct
QQSYSIPLT

SEQ ID NO: 633 - protein, 117 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIGITTPYWGQGTLVTVSS

SEQ ID NO: 634 - protein, 8 aa, synthetic construct
GFTFDDYA

SEQ ID NO: 635 - protein, 8 aa, synthetic construct
ISWNSGSI

SEQ ID NO: 636 - protein, 10 aa, synthetic construct
AKDIGITTPY

SEQ ID NO: 637 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYNYVWTFGQGTKVEIK

SEQ ID NO: 638 - protein, 6 aa, synthetic construct
QDIRND

SEQ ID NO: 639 - skipped

SEQ ID NO: 640 - protein, 9 aa, synthetic construct
LQDYNYVWT

SEQ ID NO: 641 - protein, 124 aa, synthetic construct
EVQLVESGGGLIQPGRSLRLSCAASGFTFDDYAMHWVRQAPGKGLEWVSGISWNSVSIDYADSVKGRFTISRDNAKNSLYLQMTSLTVEDTALYYCAKAFGYETLTGSDYFAYWGPGTLVTVSS

SEQ ID NO: 642 - protein, 8 aa, synthetic construct
ISWNSVSI

SEQ ID NO: 643 - protein, 17 aa, synthetic construct
AKAFGYETLTGSDYFAY

SEQ ID NO: 644 - protein, 109 aa, synthetic construct
EIVLTQSPGTLSLSPGERATLSCRASQTVISTYLAWYQQKPGQSPRLLIYGASSRATGIPDRFSGSGSGTDFTLTISRLEPEDFAVYYCQQYGSSPQLTFGGGTKVEIK

SEQ ID NO: 645 - protein, 7 aa, synthetic construct
QTVISTY

SEQ ID NO: 646 - protein, 10 aa, synthetic construct
QQYGSSPQLT

SEQ ID NO: 647 - protein, 128 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARAPGNFDWLFRSDYYYGMDVWGQGTTVTVSS

SEQ ID NO: 648 - protein, 8 aa, synthetic construct
GFTFSSYW

SEQ ID NO: 649 - protein, 8 aa, synthetic construct
IKQDGSEK

SEQ ID NO: 650 - protein, 21 aa, synthetic construct
ARAPGNFDWLFRSDYYYGMDV

SEQ ID NO: 651 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPVTFGQGTRLEIK

SEQ ID NO: 652 - protein, 6 aa, synthetic construct
QSISSY

SEQ ID NO: 653 - protein, 9 aa, synthetic construct
QQSYSTPVT

SEQ ID NO: 654 - protein, 119 aa, synthetic construct
QITLKESGPTLVKPTQTLTLTCTFSGFSLSASGVGVGWIRQPPGKALEWLALIYWDDDKRYSPSLKSRLTITKDTSKNQVVLTMTNMDPVDTATYYCAQHKVATIFDYWGQGTLVTVSS

SEQ ID NO: 655 - protein, 10 aa, synthetic construct
GFSLSASGVG

SEQ ID NO: 656 - protein, 11 aa, synthetic construct
AQHKVATIFDY

SEQ ID NO: 657 - protein, 109 aa, synthetic construct
QSALTQPPSASGSPGQSVTISCTATSSDVGNYNYVSWYQQHPGKAPKLMIYEVTKRPSGVPDRFSGSKSGNTASLTVSGLQAEDEADYYCSSYAGSWGVFGGGTKLTVL

SEQ ID NO: 658 - protein, 9 aa, synthetic construct
SSDVGNYNY

SEQ ID NO: 659 - protein, 9 aa, synthetic construct
SSYAGSWGV

SEQ ID NO: 660 - protein, 119 aa, synthetic construct
QVQLVESGGGVVQPGRSLRLSCAASGFIFSSYGMHWVRQAPGKGLEWVAVIWYDGSNKFYADSVKGRFTISRDNSKNTLYLQVNSLRAEDTAVYYCARAVQGDWYFDHWGQGTLVTVSS

SEQ ID NO: 661 - protein, 8 aa, synthetic construct
GFIFSSYG

SEQ ID NO: 662 - protein, 8 aa, synthetic construct
IWYDGSNK

SEQ ID NO: 663 - protein, 12 aa, synthetic construct
ARAVQGDWYFDH

SEQ ID NO: 664 - protein, 108 aa, synthetic construct
SYELTQPPSVSVSPGQTARITCSGDVLAKLYAYWYQHKPGQAPVLVIYKDSERPSGIPERFSGSSSGTTVTLTISGVQAEDEADYYCQSADSSGSLWVFGGGTKLTVL

SEQ ID NO: 665 - protein, 6 aa, synthetic construct
VLAKLY

SEQ ID NO: 666 - skipped

SEQ ID NO: 667 - protein, 11 aa, synthetic construct
QSADSSGSLWV

SEQ ID NO: 668 - protein, 127 aa, synthetic construct
QVQLVESGGGVVQPGRSLRLSCAASGFTFSSYGIHWVRQAPGKGLEWVAIIWYDGSEKYYADSVKGRFTISRDNSKNTLFLQMNSLEAEDTAVYYCARGPDLLTGYYNYFYYGMDVWGQGTTVTVSS

SEQ ID NO: 669 - protein, 8 aa, synthetic construct
GFTFSSYG

SEQ ID NO: 670 - protein, 8 aa, synthetic construct
IWYDGSEK

SEQ ID NO: 671 - protein, 20 aa, synthetic construct
ARGPDLLTGYYNYFYYGMDV

SEQ ID NO: 672 - protein, 111 aa, synthetic construct
QSVLTQPPSVSGAPGQRVTISCTGSSSNIGIGYDVHWYQQLPGTVPKLLIYGNTNRPSGVPDRFSGSKSGTSASLAITGLQAEDEADYYCQSYDSSLSSYVFGTGTKVTVL

SEQ ID NO: 673 - protein, 9 aa, synthetic construct
SSNIGIGYD

SEQ ID NO: 674 - skipped

SEQ ID NO: 675 - protein, 11 aa, synthetic construct
QSYDSSLSSYV

SEQ ID NO: 676 - protein, 119 aa, synthetic construct
QITLKESGPTLVKPTQTLTLTCTFSGFSLNTGGVGVGWIRQPPGNALEWLALIYWDDDKRYNPSLKTRLTISKDTSKNQVVLTMTNMDPVDTATYFCAHLTIAVIVDFWGQGTLVTVSS

SEQ ID NO: 677 - protein, 10 aa, synthetic construct
GFSLNTGGVG

SEQ ID NO: 678 - protein, 11 aa, synthetic construct
AHLTIAVIVDF

SEQ ID NO: 679 - protein, 109 aa, synthetic construct
QSALTQPASVSGSPGQSITISCTATSSDVGTYNYVSWFQHHPGKAPKLMIYEVTNRPSGVSNRFSGSKSGNTASLTISGLQAEDEADYYCSSYTTTSIVFGGGTKLTVL

SEQ ID NO: 680 - protein, 9 aa, synthetic construct
SSDVGTYNY

SEQ ID NO: 681 - protein, 9 aa, synthetic construct
SSYTTTSIV

SEQ ID NO: 682 - protein, 119 aa, synthetic construct
QITLKESGPTLVKPTQTLTLTCTFSGFSLSTSAVGVGWIRQPPGKALEWLALIYWDDDKRYSPSLKSRLTITKDTSKNQLVLTMTNVDPVDTATYYCAHHTIQLIFDYWGQGTLVTVSS

SEQ ID NO: 683 - protein, 10 aa, synthetic construct
GFSLSTSAVG

SEQ ID NO: 684 - protein, 11 aa, synthetic construct
AHHTIQLIFDY

SEQ ID NO: 685 - protein, 111 aa, synthetic construct
QSALTQPASVSGSPGQSITISCTATSSDVGNYNYVSWYQQHPGKAPKLMIYEVSNRPSGVSNRFSGSKSGNTASLTISGLQAEDEADYYCSSYTSSSTPLVFGGGTKLTVL

SEQ ID NO: 686 - protein, 11 aa, synthetic construct
SSYTSSSTPLV

SEQ ID NO: 687 - protein, 130 aa, synthetic construct
QVQLVQSGAEVKKPGASVKVSCKVSGYTLTELSIHWVRQAPGKGLEWMGGFNPEDAETIYAQKFQGRVTMTEDTSTDTAYMELRALRFEDTAVYYCATAFAVVSVGTGLNYNYYYGMDVWGQGTTVTVSS

SEQ ID NO: 688 - protein, 8 aa, synthetic construct
GYTLTELS

SEQ ID NO: 689 - protein, 8 aa, synthetic construct
FNPEDAET

SEQ ID NO: 690 - protein, 23 aa, synthetic construct
ATAFAVVSVGTGLNYNYYYGMDV

SEQ ID NO: 691 - protein, 112 aa, synthetic construct
DIVMTQTPLSSPVTLGQPASISCRSSQSLVHSDGNTYLSWLQQRPGQPPRLLIYKISNRFSGVPDRFSGSGAGTDFTLKISMVEAEDVGVYYCTQATQFPYTFGQGTNLEIK

SEQ ID NO: 692 - protein, 11 aa, synthetic construct
QSLVHSDGNTY

SEQ ID NO: 693 - skipped

SEQ ID NO: 694 - protein, 9 aa, synthetic construct
TQATQFPYT

SEQ ID NO: 695 - protein, 123 aa, synthetic construct
QVQLVQSGAEVKKPGASVKVSCKASGYTFTGYYMHWVRQAPGQGLEWMGRINPYSGGTNYAQKFQGRVTMTRDTSISTAYMELSRLRSDDTAVYYCASGFEPHDYGDPNLPPWGQGTLVTVSS

SEQ ID NO: 696 - protein, 8 aa, synthetic construct
GYTFTGYY

SEQ ID NO: 697 - protein, 8 aa, synthetic construct
INPYSGGT

SEQ ID NO: 698 - protein, 16 aa, synthetic construct
ASGFEPHDYGDPNLPP

SEQ ID NO: 699 - protein, 108 aa, synthetic construct
DIQMTQSPSSLSASVGDRVTITCRASQSISNYLNWYQQKPGKAPKLLIYAASSLQSGVPSRFSGSGSGTDFTLTISSLQPEDFATYYCQHSYSTPPVTFGGGTKVEIK

SEQ ID NO: 700 - protein, 6 aa, synthetic construct
QSISNY

SEQ ID NO: 701 - protein, 10 aa, synthetic construct
QHSYSTPPVT

SEQ ID NO: 702 - protein, 123 aa, synthetic construct
QVQLQESGPGLVKPSETLSLTCTVSGGSISGYYWSWIRQPPGKGLDWIGYIYYRGNTNYNPSLKSRVTISVDTSNNQFSLKLSSVTAADTAVYYCARYGEGYGDASGYHFDYWGQGTLVTVSS

SEQ ID NO: 703 - protein, 7 aa, synthetic construct
IYYRGNT

SEQ ID NO: 704 - protein, 17 aa, synthetic construct
ARYGEGYGDASGYHFDY

SEQ ID NO: 705 - protein, 110 aa, synthetic construct
QSALTQPASVSGSPGQSITISCTGTSSDVGSYNLVSWYQQHPGKAPKLMIYEDSKRPSGVANPFSGSKSGNTASLTISGLQAEDEADYYCSSYAGSSTWVFGGGTKLTVL

SEQ ID NO: 706 - protein, 121 aa, synthetic construct
QVQLQESGPGLVKPSETLSLTCTVSGGSISSHYWSWIRQPPGKGLEWIGYIYYSGSSNYNPSLKSRVTISVDTSKNQFSLKLNSVTAADTAVYYCARHYDILTGFDWFDPWGQGTLVTVSS

SEQ ID NO: 707 - protein, 8 aa, synthetic construct
GGSISSHY

SEQ ID NO: 708 - protein, 7 aa, synthetic construct
IYYSGSS

SEQ ID NO: 709 - protein, 15 aa, synthetic construct
ARHYDILTGFDWFDP

SEQ ID NO: 710 - protein, 112 aa, synthetic construct
QSVLTQPPSVSGAPGQRVTISCTGSSSNIGTHYDVHWYQQLPGTAPKLLIYGNSNRPSGVPDRFSGSKSGTSASLAITGLQAEDEADYYCQSFDNSLTAPYVFGTGTKVTVL

SEQ ID NO: 711 - protein, 9 aa, synthetic construct
SSNIGTHYD

SEQ ID NO: 712 - protein, 12 aa, synthetic construct
QSFDNSLTAPYV

SEQ ID NO: 713 - protein, 120 aa, synthetic construct
EVQLVESGGGLVQPGGSLRLSCAASGFTVSRSYMNWVRQAPGKGLEWVSVVFTGGTTKHTDSVKGRFTISRDNSMNTLYLQMNSLRPEDTAVYYCARVAIVETTGAFDIWGQGTMVTVSS

SEQ ID NO: 714 - protein, 7 aa, synthetic construct
VFTGGTT

SEQ ID NO: 715 - protein, 14 aa, synthetic construct
ARVAIVETTGAFDI

SEQ ID NO: 716 - protein, 112 aa, synthetic construct
QSVLTQPPSVSGAPGQRVTISCTGTSSNIGADFDVHWYQQFPGTAPKLLIYGHTNRPSGIPDRFSGSKSGTSASLAITGLQAEDEADYYCQSYDSSLSGSVVFGGGTKLTVL

SEQ ID NO: 717 - protein, 9 aa, synthetic construct
SSNIGADFD

SEQ ID NO: 718 - skipped

SEQ ID NO: 719 - protein, 119 aa, synthetic construct
QLQLQESGPGLVKPSETLSLICTGSGDSISSSHYYWGWIRQPPGKGLEWIGSTYFSGSTYYNPSLKSRVSISVDTSKNQVSLKLISVTAADTAVYYCAREMGATLGDYWGPGTLVTVSS

SEQ ID NO: 720 - protein, 10 aa, synthetic construct
GDSISSSHYY

SEQ ID NO: 721 - protein, 7 aa, synthetic construct
TYFSGST

SEQ ID NO: 722 - protein, 11 aa, synthetic construct
AREMGATLGDY

SEQ ID NO: 723 - protein, 111 aa, synthetic construct
QSVLTQPPSASGTPGQRVTISCSGSSSNIGNNYVYWYQQFPGTTPRLLIYRNNERPSGVPDRFSGSKSGTSASLVISGLRSEDEADYYCAAWDDSLSAHYVFGTGTKVTVL

SEQ ID NO: 724 - skipped

SEQ ID NO: 725 - protein, 12 aa, synthetic construct
AAWDDSLSAHYV

SEQ ID NO: 726 - protein, 126 aa, synthetic construct
EVQLVESGGGLVQPGGSLRLSCAASGFPFSTYEMNWVRQAPGKGLEWVSYISSSGNTIYYADSVKGRFTISRDNAKNSLYLQMNTLRAEDTAVYYCARDAVRDHDILTDPATFDPWGQGTLVTVSS

SEQ ID NO: 727 - protein, 8 aa, synthetic construct
GFPFSTYE

SEQ ID NO: 728 - protein, 8 aa, synthetic construct
ISSSGNTI

SEQ ID NO: 729 - protein, 19 aa, synthetic construct
ARDAVRDHDILTDPATFDP

SEQ ID NO: 730 - protein, 112 aa, synthetic construct
DIVMTQSPLSLPVTPGEPASISCRSSQTLLHSNGYNYLDWYLQKPGQSPQLLIHLGSNRASGVPDRFSGSGSVTDFTLKISRVEAEDVGVYYCMQALQTPYTFGQGTKLEIK

SEQ ID NO: 731 - protein, 11 aa, synthetic construct
QTLLHSNGYNY

SEQ ID NO: 732 - protein, 119 aa, synthetic construct
EVQLVESGGGLVQPGGSLRLSCAASGFTFSIYEMNWVRQAPGKGLEWVSYITSSGFTIYYADSVKGRFTISRDNAKNSLYLQMHSLRAEDTAVYYCATSSSSGYYFDYWGQGTLVTVSS

SEQ ID NO: 733 - protein, 8 aa, synthetic construct
GFTFSIYE

SEQ ID NO: 734 - protein, 8 aa, synthetic construct
ITSSGFTI

SEQ ID NO: 735 - protein, 12 aa, synthetic construct
ATSSSSGYYFDY

SEQ ID NO: 736 - protein, 113 aa, synthetic construct; identical to SEQ ID NO: 128

SEQ ID NO: 737 - protein, 12 aa, synthetic construct
QSVLYSSNNKNY

SEQ ID NO: 738 - skipped

SEQ ID NO: 739 - protein, 9 aa, synthetic construct
QQYYSTPPT

SEQ ID NO: 740 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYDYTWTFGQGTKVEIK

SEQ ID NO: 741 - protein, 9 aa, synthetic construct
LQDYDYTWT

SEQ ID NO: 742 - protein, 107 aa, synthetic construct
AIQMTQSPSSLSASVGDRVTITCRASQDIRNDLGWYQQKPGKAPKLLIYSASSLQTGVPSRFSGSGSGTDFTLTISSLHPEDFATYYCLQDYWYTWTFGQGTKVEIK

SEQ ID NO: 743 - protein, 9 aa, synthetic construct
LQDYWYTWT

SEQ ID NO: 744 - protein, 126 aa, synthetic construct
EVQLVESGGGLVQPGGSLRLSCAASGFTFSNHWMDWVRQAPGKGLVWVSRINSDGSSTDYADSVRGRFTMSRDNAKNTLYLQMSSLRAEDTAVYYCAREGMIFGAVIIPGWYFDLWGRGALVTVSS

SEQ ID NO: 745 - protein, 8 aa, synthetic construct
GFTFSNHW

SEQ ID NO: 746 - protein, 8 aa, synthetic construct
INSDGSST

SEQ ID NO: 747 - protein, 19 aa, synthetic construct
AREGMIFGAVIIPGWYFDL

SEQ ID NO: 748 - protein, 104 aa, synthetic construct
EIVLTQSPGTLSLSPGERATLSCRASQSVSSSQLAWYQQKPGQAPRLLIYGASSRATGIPDRFSGSGSGTDFTLTISRLEPEDFAVYYCQQYDSFGQGTKLEIK

SEQ ID NO: 749 - protein, 7 aa, synthetic construct
QSVSSSQ

SEQ ID NO: 750 - skipped

SEQ ID NO: 751 - protein, 5 aa, synthetic construct
QQYDS

SEQ ID NO: 752 - protein, 117 aa, synthetic construct
EVQLVESGGGLVQPGGSLRLSCAASGITVSSNYMTWVRQAPGKGLEWVSLIYSGGSTFYADSVRGRFTISRDNSKNTLYLQMNSLRAEDTAVYYCARDLVVYGMDVWGQGTTVTVSS